(19) United States
(12) Patent Application Publication
(10) Pub. No.: US 2002/0115727 A1
(43) Pub. Date: Aug. 22, 2002

(54) Synthesis, methods of using, and compositions of hydroxylated cyclobutylalkylamines

(76) Inventors: Chris H. Senanayake, Shrewsburry, MA (US); Paul D. Rubin, Sudbury, MA (US); Thomas P. Jerussi, Framingham, MA (US)

Correspondence Address: PENNIE & EDMONDS LLP, 1667 K STREET NW, SUITE 1000, WASHINGTON, DC, 20006

(21) Appl. No.: 09/998,195

(22) Filed: Dec. 3, 2001

Related U.S. Application Data
(60) Provisional application No. 60/250,524, filed on Dec. 4, 2000.
(60) Provisional application No. 60/257,052, filed on Dec. 22, 2000.
(60) Provisional application No. 60/250,524, filed on Dec. 4, 2000.

(51) Int. Cl. A61K 31/137; C07C 311/27; C07C 215/46

(52) U.S. Cl. 514/650; 564/99; 564/338

(57) ABSTRACT

The invention relates, in part, to making of making and using, and compositions comprising, racemic and stereomerically pure cyclobutylalkylamines, including hydroxylated sibutramine and hydroxylated metabolites of sibutramine. Methods of treating and preventing a variety of diseases and disorders are disclosed, as are pharmaceutical compositions and unit dosage forms that comprise compounds of the invention.

This application claims the benefit of U.S. Provisional Application No. 60/250,254, filed on Dec. 4, 2001, and U.S. Provisional Application No. 60/257,052, filed on Dec. 22, 2001, both of which are incorporated herein by reference.

### 1. FIELD OF THE INVENTION

The invention relates, in part, to processes for making, methods of using, and compositions comprising certain cyclobutylalkylamines, including, but not limited to, hydroxylated sibutramine and hydroxylated metabolites of sibutramine.

### 2. BACKGROUND OF THE INVENTION

Sibutramine, chemically named [N-1-[1-(4-chlorophenyl)cyclobutyl]-3-methylbutyl]-N,N-dimethylamine, is a neuronal monoamine reuptake inhibitor which was originally disclosed in U.S. Pat. Nos. 4,746,680 and 4,806,570. Sibutramine inhibits the reuptake of norepinephrine and, to a lesser extent, serotonin and dopamine. See, e.g., Buckett et al., Prog. Neuro-psychopharm. & Biol. Psychiat., 12:575-584, 1988; King et al., J. Clin. Pharm., 26:607-611 (1989).

Racemic sibutramine is sold as a hydrochloride monohydrate under the tradename MERIDIA®, and is indicated for the treatment of obesity. Physician's Desk Reference® 1509-1513 (54^{th} ed., 2000). The treatment of obesity using racemic sibutramine is disclosed, for example, in U.S. Pat. No. 5,436,272.

Sibutramine has been extensively studied, and according to such studies can be used in the treatment of a variety of disorders. Further, U.S. Pat. Nos. 4,552,828, 4,746,680, 4,806,570, and 4,929,629 disclose methods of treating depression using racemic sibutramine, and U.S. Pat. Nos. 4,871,774 and 4,939,175 disclose methods of treating Parkinson's disease and senile dementia, respectively, using racemic sibutramine. Other uses of sibutramine are disclosed by PCT publications WO 95/20949, WO 95/21615, WO 98/11884, and WO 98/13033. Further, the optically active enantiomers of sibutramine have been considered for development. For example, PCT publications WO 94/00047 and 94/00114 disclose methods of treating depression and related disorders using the (+)- and (−)-enantiomers of sibutramine, respectively.

In humans, sibutramine is rapidly absorbed from the gastrointestinal tract following oral administration and undergoes an extensive first-pass metabolism. See Jeffrey et al. J. Chem. Soc., Perkin Trans. 1, 1996, 2583-2589. This metabolism yields the primary metabolites desmethylsibutramine (DMS) and didesmethylsibutramine (DDMS) shown below.

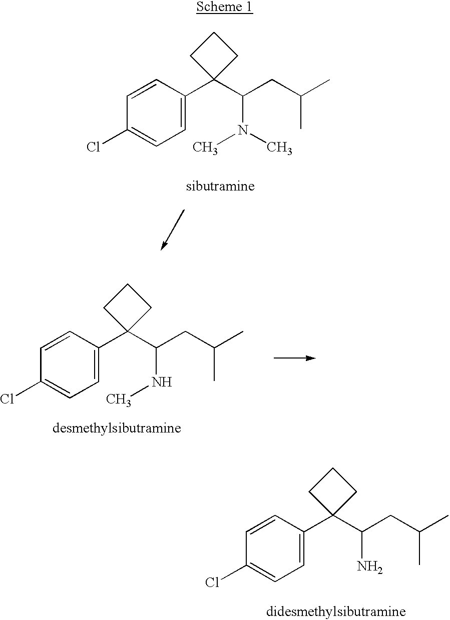

The sibutramine metabolites desmethylsibutramine and didesmethylsibutramine can each exist as an epimeric pair of R and S enantiomers as shown below:

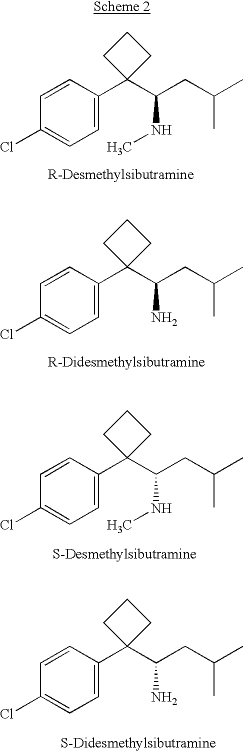

It has been reported that the primary metabolites of sibutramine, desmethylsibutramine and didesmethylsibutramine, are more potent in vitro noradrenaline and 5-hydroxytryptamine (5HT; serotonin) reuptake inhibitors than sibutramine. Stock, M. J., Int'l J. Obesity, 21(Supp. 1):S25-S29 (1997); See also Luscombe et al. Neuropharmacology Vol. 28, No. 2, 1989, pp. 129-134. It has further been reported, however, that sibutramine and its primary metabolites have negligible affinities for a wide range of neurotransmitter receptors, including serotonergic (5-HT₁, 5-HT_{1A}, 5-HT_{1D}, 5-HT_{2A}, 5-HT_{2C}), adrenergic, dopaminergic, muscarinic, histaminergic, glutamate, and benzodiazepine receptors. Id.

The existence of other sibutramine metabolites has been reported in mice. See Jeffrey et al. J. Chem. Soc., Perkin Trans. 1, 1996, 2583-2589. For example, oxidative metabolism reportedly yields two hydroxylated amines; the synthesis of the racemic 7-hydroxyl-amine below has been described. Id.

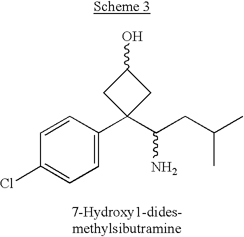

Sibutramine has been reported to exhibit a variety of adverse effects. See, e.g., Physician's Desk Reference® 1509-1513 (54^{th} ed., 2000). Coupled with the reported benefits and therapeutic insufficiencies of sibutramine, this fact has encouraged the discovery of compounds and compositions that can be used in the treatment or prevention of disorders such as, but not limited to, obesity, depression, and related disorders. In particular, compounds and compositions are desired that can be used for the treatment and prevention of such and other disorders and conditions while incurring fewer or avoiding adverse side-effects associated with sibutramine administration.

### 3. SUMMARY OF THE INVENTION

This invention encompasses novel compounds (including stereomerically pure isomers) and pharmaceutical compositions for the treatment and prevention of diseases and/or disorders that are ameliorated by the inhibition of neuronal monoamine uptake in mammals. Examples of such diseases and/or disorders include, but are not limited to, eating disorders, weight gain, or obesity; irritable bowel syndrome; obsessive-compulsive disorders; platelet adhesion; apnea; affective disorders (e.g., ADHD), depression, or anxiety; male or female sexual function disorders, such as erectile dysfunction; restless leg syndrome; osteoarthritis; substance abuse including, nicotine addiction from cigarette smoking or chewing tobacco, and cocaine addiction; narcolepsy; pain, neuropathic pain, diabetic neuropathy, chronic pain; migraines; cerebral function disorders; chronic disorders; premenstrual syndrome; and incontinence. The invention also encompasses methods of treating and preventing diseases and conditions, which comprise administering to a patient in need of such treatment or prevention a therapeutically or prophylactically effective amount of a sibutramine-based compound.

The sibutramine-based compounds of the invention include, but are not limited to, racemates, other mixtures, and stereomerically pure compounds. The invention is also directed to pharmaceutical compositions and dosage forms that comprise therapeutically or prophylactically effective amounts of the compounds, optionally in combination with an additional pharmacologically active compound. Further, the invention includes pharmaceutically acceptable solvates, including hydrates; anhydrous compounds; and clathrates. Yet further, the invention includes pharmaceutically acceptable salts of these solvates, hydrates, anhydrous compounds and the like. Finally, the invention includes esters and prodrugs of compounds of the invention. The universe of compounds encompassed by the invention may be referred to herein as “compounds of the invention.”

The invention further encompasses methods of synthesizing hydroxylated sibutramine-based compounds, as well as, intermediates and isomers and mixtures thereof, including racemates and stereomerically pure compounds.

In a preferred embodiment, the pharmaceutical compositions of the invention comprise a therapeutically or prophylactically effective amount of a racemic or stereomerically pure sibutramine-based compound, including mixtures thereof, and pharmaceutically acceptable salts, solvates, hydrates, esters, clathrates, and prodrugs thereof. In another embodiment, the pharmaceutical compositions of the invention can further comprise other drug substances including, but not limited to, 5-HT₃ antagonists, lipase inhibitors for obesity or weight management, apomorphine, or a phosphodiesterase inhibitor.

The invention encompasses the use of a racemic or stereomerically pure sibutramine-based compounds, enantiomeric or diastereomeric mixtures thereof, or pharmaceutically acceptable salts, solvates, hydrates, esters, clathrates, and prodrugs thereof as effective dopamine, serotonin, and norepinephrine reuptake inhibitors.

More specifically, in one embodiment the invention encompasses novel racemic and stereomerically pure cyclobutylalkylamines as shown below:

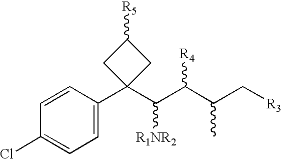

wherein R₁ and R₂ are indpendendently a hydrogen or an alkyl group and R₃, R₄, and R₅ are independently a hydrogen, a hydroxyl group, or an alkoxyl group and at least one of R₃, R₄, and R₅ is a hydroxyl group or a alkoxyl group with maximum of three hydroxyl or alkoxyl groups.

As used herein and unless otherwise indicated, bonds drawn as wavy lines or single lines may represent stereochemistry in a structure or a portion of a structure and if not indicated with, for example, bold or dashed lines, the structure or portion of the structure is to be interpreted as encompassing all stereoisomers of it.

Specific examples, of novel cyclobutylalkylamine compounds encompassed by the invention include, but are not limited to, racemic and stereomerically pure I -hydroxylated sibutramine, racemic and stereomerically pure 1-hydroxylated desmethylsibutramine, stereomerically pure 1-hydroxylated didesmethylsibutramine (Scheme 4); racemic and stereomerically pure 3-hydroxylated sibutramine, racemic and stereomerically pure 3-hydroxylated desmethylsibutramine, racemic and stereomerically pure 3-hydroxylated didesmethylsibutramine (Scheme 5); and racemic and stereomerically pure 7-hydroxylated sibutramine, racemic and stereomerically pure 7-hydroxylated desmethylsibutramine, and stereomerically pure 7-hydroxylated didesmethylsibutramine (Scheme 6).

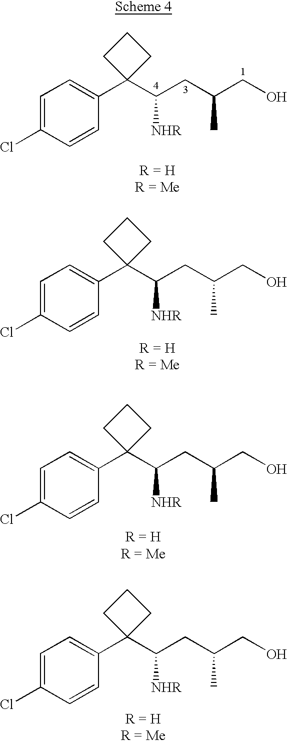

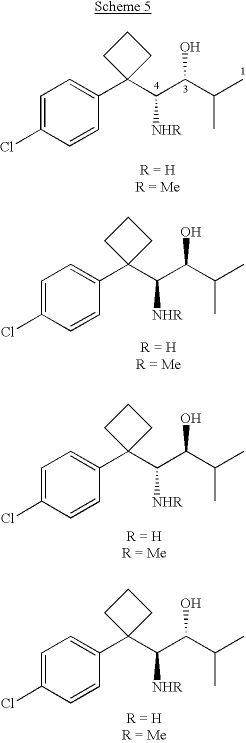

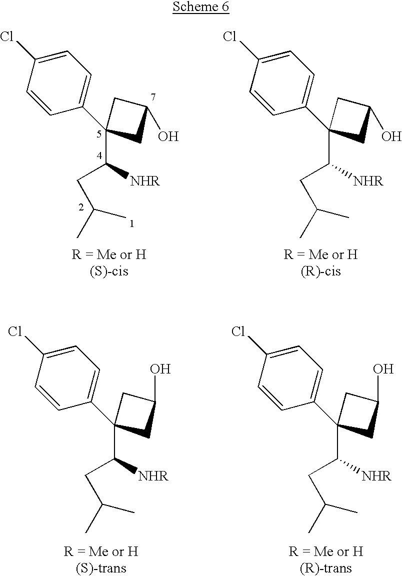

Finally, the invention encompasses novel and efficient methods, including asymmetric methods, for synthesizing hydroxylated sibutramine and hydroxylated desmethyl- and didesmethyl-sibutramine, including novel compounds.

3.1. Definitions

As used herein the terms “sibutramine-based compounds” and “derivatives of cyclobutylalkylamine compounds” are used interchangeably and refer to compounds of the formula:

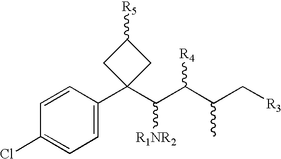

wherein each of R₁ and R₂ is independently lower alkyl or hydrogen, and each of R₃, R₄, and R₅ is independently a hydrogen, hydroxyl, or alkoxy, or a pharmaceutically acceptable salt, solvate, hydrate, clathrate, ester, or prodrug thereof. Preferably, at least one of R₃, R₄, and R₅ is not hydrogen. It is also preferred that if R₁, R₂, R₄, and R₅ are each hydrogen and R₃ is hydroxyl, the compound is not racemic, and if R₁, R₂, R₃, and R₄ are each hydrogen and R₅ is hydroxyl, the compound is not racemic.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it. Furthermore, a chemical structure drawn with a wavy line as a bond indicates that the structure shown encompasses all possible stereochemistries at that bond.

As used herein, the term “hydroxylated sibutramine metabolite” refers to a hydroxylated sibutramine-based compounds. Hydroxylated sibutramine metabolites include, but are not limited to, hydroxylated sibutramine-based compounds, wherein the hydroxyl is in a position to form a primary, secondary or tertiary hydroxylated sibutramine-based compound. In a particular embodiment, the hydroxylated sibutramine metabolite is a 1-hydroxyl, 3-hydroxyl, or 7-hydroxyl sibutramine metabolite or a polyhydroxylated sibutramine metabolite as shown herein or mixture thereof. As used herein, the term “hydroxylated sibutramine” refers to sibutramine that is hydroxylated in any position to form a primary, secondary or tertiary hydroxylated sibutramine or polyhydroxylated sibutramine. In a particular embodiment, the hydroxylated sibutramine is I-hydroxyl, 3-hydroxyl, or 7-hydroxyl sibutramine as shown herein.

As used herein and unless otherwise indicated, the term “alkyl” or “alkyl group” includes saturated monovalent linear, branched, substituted, and cyclic hydrocarbon radicals, including aryl groups. An alkyl group can include one or more double or triple bonds. It is understood that cyclic alkyl groups comprise at least three carbon atoms. Preferred alkyl groups include, but are not limited to, branched or linear alkyl having from 1 to 6, more preferably from 1 to 4 carbon atoms. Examples include, but are not limited to, methyl, ethyl, propyl, isopropyl, isobutyl, and tertiary butyl.

As used herein and unless otherwise indicated, the term “substituted” as used to describe a compound or chemical moiety means that at least one hydrogen atom of that compound or chemical moiety is replaced with a second chemical moiety. Examples of second chemical moieties include, but are not limited to: halogen atoms (e.g., chlorine, bromine, and iodine); C₁-C₆ linear, branched, or cyclic alkyl (e.g., methyl, ethyl, butyl, tert-butyl, and cyclobutyl); hydroxyl; thiols; carboxylic acids; esters, amides, silanes, nitriles, thioethers, stannanes, and primary, secondary, and tertiary amines (e.g., —NH₂, —NH(CH₃), —N(CH₃)₂, and cyclic amines). Preferred second chemical moieties are chlorine, hydroxyl, methoxy, amine, thiol, and carboxylic acid.

As used herein and unless otherwise indicated, the term “aryl” includes an organic radical derived from an aromatic hydrocarbon by removal of one hydrogen, such as phenyl or naphthyl.

As used herein and unless otherwise indicated, the term “alkoxyl” or “alkoxyl group” refers to the group —OR, wherein O is oxygen and R is an alkyl as described above. Preferred alkoxyl groups include, but are not limited to, branched or linear alkoxyl groups having from 1 to 6, more preferably from 1 to 4 carbon atoms. Examples include, but are not limited to, methoxy, ethoxy, propoxy, isopropoxy, isobutoxy, and tertiary butoxy.

As used herein and unless otherwise indicated, a composition that is “substantially free” of a compound means that the composition contains less than about 20% by weight, more preferably less than about 10% by weight, even more preferably less than about 5% by weight, and most preferably less than about 3% by weight of the compound.

As used herein and unless otherwise indicated, the terms “stereomerically pure,” and “optically pure” are used interchangeably to mean a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of stereoisomer of the compound and less than about 20% by weight of other stereoisomers the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

For example, in one embodiment the invention encompasses stereomerically pure S-cis-7-hydroxylated desmethylsibutramine, which is substantially free of R-cis-7-hydroxylated desmethylsibutramine, S-trans-7-hydroxylated desmethylsibutramine, and R-trans-7-hydroxylated desmethylsibutramine. Another example of an embodiment the invention encompasses (2R,4R)-1-hydroxylated desmethylsibutramine substantially free from (2S,4R)-1-hydroxylated desmethylsibutramine, (2S,4S)-1-hydroxylated desmethylsibutramine, and (2R,4S)-1-hydroxylated desmethylsibutramine. Still another example of an embodiment the invention encompasses (3R,4R)-3-hydroxylated desmethylsibutramine substantially free from (3S,4R)-3-hydroxylated desmethylsibutramine, (3S,4S)-3-hydroxylated desmethylsibutramine, and (3R,4S)-3-hydroxylated desmethylsibutramine. Typical stereomerically pure compounds of the invention are optically active.

As used herein and unless otherwise indicated, the term “enantiomerically pure” means a stereomerically pure composition of a compound having one chiral center.

As used herein, the term “prodrug” means a derivative of an active compound that can hydrolyze, oxidize, or otherwise react under biological conditions (in vitro or in vivo) to provide the active compound. Examples of prodrugs include, but are not limited to, derivatives of hydroxylated didesmethylsibutramine having biohydrolyzable moieties such as biohydrolyzable amides, biohydrolyzable esters, biohydrolyzable carbamates, biohydrolyzable carbonates, and biohydrolyzable ureides. As used herein, prodrugs of hydroxylated didesmethylsibutramine, for example, do not include hydroxylated sibutramine or metabolites of sibutramine and do not include sibutramine, desmethylsibutramine, or didesmethylsibutramine.

As used herein, the terms “biohydrolyzable carbamate,” “biohydrolyzable carbonate,” and “biohydrolyzable ureide” mean a carbamate, carbonate, or ureide, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the term “biohydrolyzable ester” means an ester of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, alkoxyacyloxy esters, alkyl acylamino alkyl esters, and choline esters.

As used herein, the term “biohydrolyzable amide” means an amide of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable amides include, but are not limited to, lower alkyl amides, α-amino acid amides, alkoxyacyl amides, and alkylaminoalkylcarbonyl amides.

As used herein, the term “pharmaceutically acceptable salt” refers to a salt prepared from a pharmaceutically acceptable non-toxic inorganic or organic acid. Inorganic acids include, but are not limited to, hydrochloric, hydrobromic, hydroiodic, nitric, sulfuric, and phosphoric. Organic acids include, but are not limited to, aliphatic, aromatic, carboxylic, and sulfonic organic acids including, but not limited to, formic, acetic, propionic, succinic, benzoic camphorsulfonic, citric, fumaric, gluconic, isethionic, lactic, malic, mucic, tartaric, para-toluenesulfonic, glycolic, glucuronic, maleic, flroic, glutamic, benzoic, anthranilic, salicylic, phenylacetic, mandelic, embonic (pamoic), methanesulfonic, ethanesulfonic, pantothenic, benzenesulfonic, stearic, sulfanilic, alginic, and galacturonic acid.

### 4. DETAILED DESCRIPTION OF THE INVENTION

4.1. Compounds

The invention encompasses sibutramine-based compounds, methods of their synthesis, and methods of their use. A first embodiment of the invention encompasses racemic or stereomerically pure mono, di, and tri-hydroxylated sibutramine compounds as shown below:

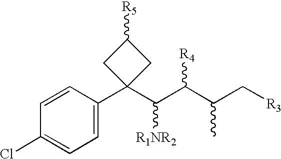

wherein each of R₁ and R₂ is independently lower alkyl or hydrogen, and each of R₃, R₄, and R₅ is independently hydrogen, hydroxyl, or alkoxy provided that at least one of R₃, R₄, and R₅ is not hydrogen, and pharmaceutically acceptable salts, solvates, hydrates, clathrate, prodrug thereof. In a specific embodiment, if R₁, R₂, R₄, and R₅ are each hydrogen and R₃ is hydroxyl, the compound is not racemic. In another specific embodiment, if R₁, R₂, R₃, and R₄ are each hydrogen and R₅ is hydroxyl, the compound is not racemic.

For example, the invention encompasses racemic or stereomerically pure 1, 3, and 7 hydroxylated sibutramine compounds as shown below:

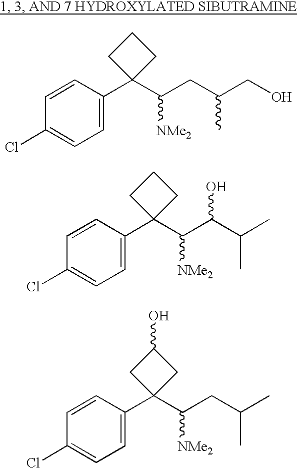

A preferred embodiment of the invention encompasses stereomerically pure sibutramine-based compounds that are hydroxylated in the 1-position as shown below:

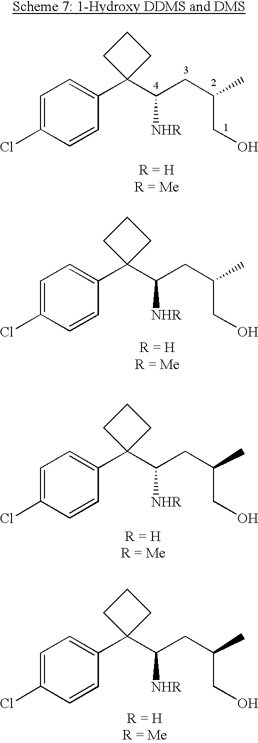

Another embodiment encompasses racemic and stereomerically pure 3-hydroxylated sibutramine-based compounds as shown below:

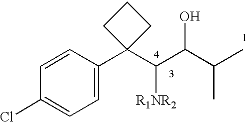

wherein each of R₁ and R₂ is independently hydrogen or alkyl or enantiomeric and diastereomeric mixtures of 3-hydroxyl desmethylsibutramine and enantiomeric and diastereomeric mixtures of 3-hydroxy didesmethylsibutramine, respectively.

In a particular embodiment, the invention encompasses stereomerically pure 3-hydroxyl desmethylsibutramine isomers and 3-hydroxyl didesmethylsibutramine isomers as shown below:

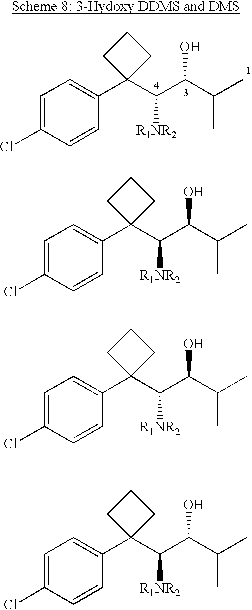

wherein each of R₁ and R₂is independently hydrogen or alkyl or enantiomeric and diastercomeric mixtures thereof.

When R₁ is Me (i.e., methyl) and R₂ is hydrogen the compounds include a hydroxylated secondary amine metabolite of sibutramine, i.e., 3-hydroxy-desmethylsibutramine. When R₁ and R₂ are both H the compounds include a hydroxylated primary amine metabolite of sibutramine, i.e., 3-hydroxy-didesmethylsibutramine.

In another embodiment, the invention relates to racemic and stereomerically pure 7-hydroxylated desmethylsibutramine as shown below:

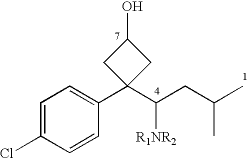

wherein each of R₁ and R₂ is independently hydrogen or alkyl or enantiomeric and diastereomeric mixtures thereof.

In a particular embodiment, the invention encompasses stereomerically pure 7-hydroxylated sibutramine metabolites as shown below:

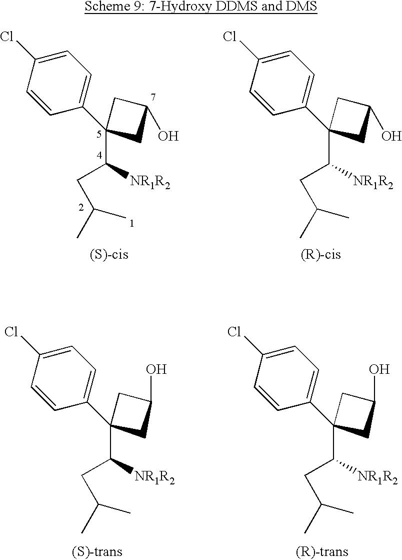

wherein each of R₁ and R₂ is independently hydrogen or alkyl or enantiomeric and diastereomeric mixtures thereof, which includes its cis and trans isomers and mixtures thereof.

The invention also encompasses mixtures of stereoisomers, which include mixtures of diastereomers and mixtures of enantiomers. For example, each mono-hydroxylated compound of the invention (e.g., 1-hydroxyl-desmethylsibutramine) can exist as one of four possible stereoisomers, i.e., 1-hydroxyl-desmethylsibutramine can exist as (R,R)-1-hydroxyl-desmethylsibutramine, (S,S)-1-hydroxyl-desmethylsibutramine, (R,S)-1-hydroxyl-desmethylsibutramine, (S,R)-1-hydroxyl-desmethylsibutramine, or mixtures thereof. As such, the invention encompasses stereomerically pure compounds, as defined herein, as well as, any stereomeric mixtures including mixtures of enantiomers or diastereoisomers. For example, mixtures include, but are not limited to, varying amounts of (S,S), (R,R), (S,R), (R,S) orientations and the like. Preferred mixtures are not racemic.

4.2. Pharmaceutical Compositions

The invention encompasses pharmaceutical compositions and unit dosage forms comprising a racemic or stereomerically pure sibutramine-based compound, preferably hydroxylated in the 1-position, the 3-position, or the 7-position as described herein, or a pharmaceutically acceptable salt, solvate, hydrate, ester clathrate, or prodrug thereof. Stereomerically pure sibutramine-based compounds are most preferred.

The invention also encompasses pharmaceutical compositions and dosage forms which comprise diastereomeric and enantiomeric mixtures of a sibutramine-based compound, and diastereomeric or enantiomeric mixtures of 1-hydroxylated, 3-hydroxylated, and 7-hydroxylated sibutramine-based compounds, respectively.

These pharmaceutical compositions and dosage forms are particularly useful in the methods described herein. For example, the pharmaceutical compositions and dosage forms of the invention are suitable for oral, mucosal (e.g., nasal, sublingual, buccal, rectal, and vaginal), parenteral (e.g., intravenous, intramuscular or subcutaneous), or transdermal administration. In a preferred embodiment, the pharmaceutical compositions and dosage forms comprise a racemic or stereomerically pure sibutramine-based compound, in an amount from about 0.01 mg to about 500 mg, preferably from about 0.1 mg to about 250 mg, more preferably from about, and even more preferably from about 1 mg to about 100 mg.

Pharmaceutical compositions and dosage forms of the invention comprise one or more of the sibutramine-based compounds disclosed herein (e.g., 1-hydroxyl desmethylsibutramine, or a pharmaceutically acceptable prodrug, ester, salt, solvate, hydrate, or clathrate thereof). Pharmaceutical compositions and dosage forms of the invention typically also comprise one or more pharmaceutically acceptable excipients or diluents. Specific compounds and pharmaceutical compositions can further comprise a second therapeutically or prophylactically active compound as set forth herein (e.g., in Section 4.4).

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of disorder may contain larger amounts of one or more of the active ingredients it comprises than a dosage form used in the chronic treatment of the same disorder. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients, such as, hydroxylated desmethyl and didesmethyl-sibutramine and its stereomerically pure enantiomers and diastereomers in particular, can be accelerated by some excipients such as lactose, or when exposed to water. Active ingredients that comprise primary or secondary amines (e.g., 1-hydroxyl desmethylsibutramine and its stereomerically pure enantiomers and diastereomers) are particularly susceptible to such accelerated decomposition. Consequently, this invention encompasses pharmaceutical compositions and dosage forms that contain little, if any, lactose or mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmocopia (USP) SP (XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise a racemic or optically pure sibutramine-based compound or a pharmaceutically acceptable salt, solvate, clathrate, hydrate, or prodrug thereof in an amount of from about 0.01 mg to about 500 mg, preferably in an amount of from about 0.1 mg to about 250 mg, and more preferably in an amount of from about 1 mg to about 100 mg.

4.2.1. Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, fillers, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103Tm and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The magnitude of a prophylactic or therapeutic dose of an active ingredient in the acute or chronic management of a disorder or condition will vary with the severity of the disorder or condition to be treated and the route of administration. The dose, and perhaps the dose frequency, will also vary according to age, body weight, response, and the past medical history of the patient. Suitable dosing regimens can be readily selected by those skilled in the art with due consideration of such factors.

The dosage amounts and frequencies provided above are encompassed by the terms “therapeutically effective,” “prophylactically effective,” and “therapeutically or prophylactically effective” as used herein. When used in connection with an amount of a racemic or optically pure sibutramine metabolite, these terms further encompass an amount of racemic or optically pure sibutramine metabolite that induces fewer or less sever adverse effects than are associated with the administration of racemic sibutramine. Adverse effects associated with racemic sibutramine include, but are not limited to, significant increases in supine and standing heart rate, including tachycardia, increased blood pressure (hypertension), increased psychomotor activity, dry mouth, dental caries, constipation, hypohidrosis, blurred or blurry vision, tension, mydriasis, seizures, formation of gallstones, renal/hepatic dysfunction, fevers, arthritis, agitation, leg cramps, hypertonia, abnormal thinking, bronchitis, dyspnea, pruritus, amblyopia, menstrual disorder, ecchymosis/bleeding disorders, interstitial nephritis, and nervousness. See, e.g., Physician's Desk Reference® 1494-1498 (53^{rd} ed., 1999).

4.2.2. Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.2.3. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

4.2.4. Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.2.5. Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a unit dosage form of a racemic or stereomerically pure sibutramine-based compound or a pharmaceutically acceptable prodrug, salt, solvate, hydrate, or clathrate thereof, and a unit dosage form of a second active ingredient. Examples of second active ingredients include, but are not limited to, 5-HT₃ antagonists, apomorphine, phosphodiesterase inhibitors, and lipase inhibitors for obesity and weight management.

Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

4.3. Methods of Use

The invention is based, in part, on the discovery that sibutramine-based compounds and racemic and stereomerically pure isomers thereof, can be used for the treatment and prevention of disorders that are ameliorated by the inhibition of neuronal monoamine uptake.

As such, the invention encompasses a method of treating or preventing a disorder and/or disease ameliorated by the inhibition of neuronal monoamine uptake which comprises administering to a patient in need of such treatment or prevention a therapeutically or prophylactically effective amount of 1-hydroxy, 3-hydroxy, or 7-hydroxy sibutramine compound (e.g., hydroxylated desmethyl- or didesmethylsibutramine), or a pharmaceutically acceptable salt, solvate, hydrate, ester, clathrate, or prodrug thereof. In a preferred embodiment, the disorder and condition ameliorated by inhibition of neuronal monoamine uptake is an eating disorder, weight gain, or obesity; platelet adhesion; apnea; obsessive-compulsive disorders; affective disorders (e.g., ADHD), depression, or anxiety; male and female sexual function disorders, such as erectile dysfunction; restless leg syndrome; osteoarthritis; irritable bowel syndrome; substance abuse including, nicotine addiction from cigarette smoking or chewing tobacco, and cocaine addiction; migraines; chronic pain; pain, such as neuropathic pain, such as diabetic neuropathy; cerebral function disorders; chronic disorders; and incontinence. The patients include mammals, particularly humans and also includes dogs, cats, and feedstock.

In a preferred embodiment the hydroxyl group is selectively substituted in the 1-position, the 3-position, or the 7-position, to form a compound as illustrated below:

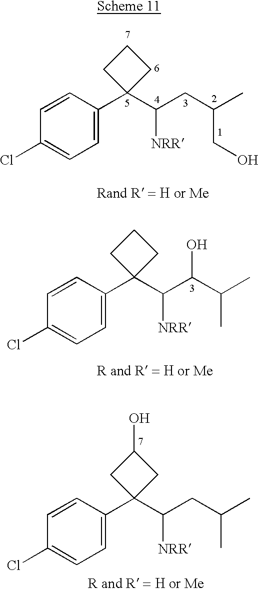

The “stereomerically pure” isomers of these compounds can also be synthesized or otherwise isolated and their use in the methods or compositions of the invention is contemplated.

As used herein, the term “treating or preventing disorders ameliorated by inhibition of neuronal monoamine reuptake” means relief from symptoms of conditions associated with abnormal neuronal monoamine levels.

Another embodiment of the invention encompasses a method of treating or preventing male or female sexual function disorders, which comprises administering to a patient in need of such treatment or prevention a therapeutically or prophylactically effective amount of a racemic or stereomerically pure sibutramine-based compound, or a pharmaceutically acceptable salt, solvate, hydrate, ester, clathrate, or prodrug thereof. As used herein, the terms “sexual dysfunction” and “sexual function disorder” encompass sexual dysfunction in men and women caused by psychological and/or physiological factors. Examples of sexual dysfunction include, but are not limited to, sexual arousal disorder, erectile dysfunction, vaginal dryness, lack of sexual excitement, orgasmic disorder, or inability to obtain orgasm. The term “sexual dysfunction” further encompasses psycho-sexual dysfunction. Examples of psycho-sexual dysfunction include, but are not limited to, hypoactive sexual desire disorder, sexual aversion disorders, inhibited sexual desire, inhibited sexual excitement, inhibited female orgasm, inhibited male orgasm, premature ejaculation, functional dyspareunia, functional vaginismus, and atypical psychosexual dysfunction. In a preferred method of this embodiment, the racemic or stereomerically pure sibutramine-based compound, or pharmaceutically acceptable salt, solvate, hydrate, ester, clathrate, or prodrug thereof is administered orally, transdermally, or mucosally. In a particular embodiment, the sibutramine-based compound is hydroxylated in the 1-position, the 3-position, or the 7-position. The treatment or prevention of sexual dysfunction in elderly or postmenstrual patients is also included. The prevention of sexual dysfunction disorder involves recognition by one of skill in the art of that population at risk of sexual dysfunction disorder. In particular, one of skill in the art will recognize those at risk of sexual dysfunction disorder and in need of prevention to include, but not limited to, individuals suffering from: psychological problems, for example, anxiety over sexual intercourse, guilt after a pleasurable experience, shame, fear of intimacy, depression, ignorance of sexual norms, or frustration; situational factors, for example, marital discord, boredom, or negative emotions; or physical factors.

Another embodiment of the invention encompasses a method of treating or preventing an affective disorder which comprises administering to a patient in need of such treatment or prevention a therapeutically or prophylactically effective amount of a racemic or stereomerically pure sibutramine-based compound, or a pharmaceutically acceptable salt, solvate, hydrate, ester, clathrate, or prodrug thereof Affective disorders include, but are not limited to, depression (e.g., melancholia), attention deficit disorder (including attention deficit disorder with hyperactivity and attention deficit/hyperactivity disorder), bipolar and manic conditions, dysthymic disorder, and cyclothymic disorder. As used herein, the terms “attention deficit disorder” (ADD), “attention deficit disorder with hyperactivity” (ADDH), and “attention deficit/hyperactivity disorder” (AD/HD), are used in accordance with their accepted meanings in the art. See, e.g., Diagnostic and Statistical Manual of Mental Disorders, Fourth Ed., American Psychiatric Association, 1997 (DSM-IV™) and Diagnostic and Statistical Manual of Mental Disorders, 3^{rd} Ed., American Psychiatric Association (1981) (DSM-III™).

A preferred method of this embodiment is a method of treating or preventing attention deficit disorder which comprises administering to a patient in need of such treatment or prevention a therapeutically or prophylactically effective amount of racemic or stereomerically pure sibutramine-based compound, or a pharmaceutically acceptable salt, solvate, hydrate, ester, clathrate, or prodrug thereof. In the treatment or prevention of attention deficit disorder, the sibutramine-based compound is stereomerically pure, and more preferably the stereomerically pure sibutramine-based compound is hydroxylated in the 1-position, the 3-position, or the 7-position. In a particular embodiment, the method can also be used to treat or prevent a condition in children (e.g., ages 3-18). One of skill in the art will recognize those at risk of attention deficit disorders and in need of prevention of such include, but not limited to, individuals who, for example, fail to maintain close attention, fail to listen, fail to finish tasks, avoid sustained mental effort, are distracted by extraneous stimuli, talk excessively, or interrupts or intrudes on others.

Another preferred method of this embodiment is a method of treating or preventing depression which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof. As used herein, the term “treating or preventing depression” means relief from or prevention of the symptoms of depression which include, but are not limited to, changes in mood, feelings of intense sadness, despair, mental slowing, loss of concentration, pessimistic worry, agitation, and self-deprecation. Physical changes can also be relieved or prevented by this method, and include, but are not limited to, insomnia, anorexia, decreased energy and libido, and abnormal hormonal circadian rhythms. One of skill in the art will recognize those at risk of depression and in need of prevention of such disorder to include, but not limited to, individuals who, for example, appear miserable, with furrowed brows, down-turned corners of the mouth, slumped posture, poor eye contact, and monosyllabic speech. These activities may be accompanied by preoccupation with guilt, self-denigrating ideas, decreased ability to concentrate, indecisiveness, diminished interest in usual activities, social withdrawal, helplessness, hopelessness, recurrent thoughts of death or suicide or combinations thereof.

Another embodiment of the invention encompasses a method of treating or preventing weight gain or obesity which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof. As used herein, the term “treating or preventing weight gain or obesity” means reduction of weight, relief from being overweight, treating weight gain caused by the administration of other drugs, relief from gaining weight, or relief from obesity, and prevention from gaining weight, all of which are usually due to unnecessary consumption of food. The invention also encompasses methods of treating or preventing conditions incidental to obesity including, but not limited to, hypertension, such as pulmonary hypertension; cancers, such as breast, colon, gall bladder, and endometrial; gall stones; cardiovascular disease, such as dyslipidemia and carotid intimal medial thickening; hiatial hernia; osteoarthritis; gout; thyroid disease, such as diabetes; gastro-esophogeal reflux disease; menstrual dysfunction; and infertility. In a particular embodiment, the racemic or stereomerically pure sibutramine-based compound or a racemic is hydroxylated in the 1-position, the 3-position, or the 7-position. In a particular method of this embodiment, the weight gain is associated with the administration of a drug that induces weight gain. In another method of this embodiment, the weight gain is associated with smoking cessation.

Another embodiment encompasses a method of treating or preventing a disorder associated with the administration of a lipase inhibitor for obesity or weight management, such as, for example, orlistat (XENICAL®), which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof. As used herein, the term “treating or preventing a disorder associated with the administration of a lipase inhibitor” means alleviating or reducing adverse effects associated with administration of a lipase inhibitor, which include, but are not limited to, infectious diarrhea, oily fecal spotting, flatus with discharge, fecal urgency, fatty/oily stool, oily evacuation, increased defecation, anal leakage, and fecal incontinence.

Another embodiment encompasses a method of treating or preventing cerebral function disorders which comprises administering to a patient in need of such treatment or prevention a therapeutically or prophylactically effective amount of a racemic or stereomerically pure sibutramine-based compound or a pharmaceutically acceptable salt, solvate, hydrate, or clathrate thereof. Cerebral function disorders include, but are not limited to, senile dementia, Alzheimer's type dementia, memory loss, amnesia/amnestic syndrome, disturbance of consciousness, coma, lowering of attention, speech disorders, Parkinson's disease, Lennox syndrome, autism, epilepsy, hyperkinetic syndrome, and schizophrenia. Cerebral function disorders can be induced by factors including, but not limited to, cerebrovascular diseases, such as cerebral infarction, cerebral bleeding, cerebral arteriosclerosis, cerebral venous thrombosis, and head injuries, and conditions having symptoms selected from the group consisting of disturbances of consciousness, senile dementia, coma, lowering of attention, and speech disorders. As used herein, the term “treating or preventing a cerebral function disorder” means relief from or prevention of one or more symptoms associated with cerebral function disorders. One of skill in the art will recognize those at risk of cerebral function disorders and in need of prevention of such disorders include, but are not limited to, individuals who, for example, exhibit dementia, memory loss, amnesia/amnestic syndrome, disturbance of consciousness, coma, lowering of attention, speech disorders, autism, epilepsy, hyperkinetic syndrome, and schizophrenia.

Another embodiment encompasses a method of treating or preventing restless leg syndrome, which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof. As used herein, the term “restless leg syndrome” encompasses a disorder that typically occurs during sleep or rest and is characterized by uncomfortable sensations in the legs, which include, but are not limited to, pulling, drawing, crawling, wormy, boring, tingling, pins and needles, prickly and sometimes painful sensations that are usually accompanied by an overwhelming urge to move the legs. As used herein, the term “restless leg syndrome” also encompasses Ekbom Syndrome, Wittmaack-Ecbom Syndrome, Hereditary Acromelalgia, and Anxieties Tibialis.

Another embodiment encompasses a method of treating or preventing pain which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof. In a particular embodiment, the pain is chronic pain, such as neuropathic pain, such as diabetic neuropathy.

Still another embodiment of the invention encompasses a method of treating or preventing obsessive-compulsive disorder which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof. One of skill in the art will recognize those at risk of or predisposed to obsessive-compulsive disorder and in need of prevention of such disorders include, but are not limited to, individuals who, for example, feel compelled to perform repetitive, purposeful, intentional behaviors called rituals to balance their obsessions. As used herein, the terms “obsessive-compulsive disorder,” “pre-menstrual syndrome,” “anxiety,” and “eating disorder” are used consistently with their accepted meanings in the art. See, e.g., DSM-IV™ and DSM-III™. The term “methods of treating or preventing” when used in connection with these disorders means the amelioration, prevention, or relief from symptoms and/or effects associated with these disorders.

Another embodiment encompasses a method of treating or preventing substance abuse which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof. In a particular embodiment, the substance abuse is cocaine addiction or alcohol addiction. As used herein, the term “substance abuse” encompasses the abuse of, and physical and/or psychological addiction to, drugs or alcohol. The term “substance abuse” further encompasses its accepted meaning in the art. See, e.g., DSM-IV™ and DSM-III™. A preferred method encompassed by this embodiment is a method of treating or preventing cocaine and/or heroin abuse. One of skill in the art will recognize those at risk of or predisposed to substance abuse and in need of prevention of such include, but are not limited to, individuals who, for example, are frequent users of drugs or alcohol.

Another embodiment encompasses a method of treating or preventing nicotine addiction which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, prodrug thereof. Nicotine addiction includes nicotine addiction of all known forms, such as smoking cigarettes, cigars and/or pipes, and addiction to chewing tobacco.

Another embodiment encompasses a method of eliciting smoking cessation which comprises administering to a patient who smokes tobacco a therapeutically effective amount of a racemic or stereomerically pure sibutramine-based compound or a pharmaceutically acceptable salt, solvate, hydrate, ester, clathrate, or prodrug thereof. In a preferred method encompassed by this embodiment, the racemic or stereomerically pure sibutramine-based compound or pharmaceutically acceptable salt, solvate, hydrate, ester, clathrate, or prodrug thereof is administered orally, mucosally, or transdermally. In a more preferred method, it is administered transdermally.

Another preferred method encompassed by this embodiment is a method of eliciting smoking cessation which comprises adjunctively administering to a patient who smokes tobacco a therapeutically or prophylactically effective amounts of a racemic or stereomerically pure sibutramine-based compound, or a pharmaceutically acceptable salt, solvate, hydrate, ester, clathrate, or prodrug thereof, and nicotine. Preferably, the nicotine and/or racemic or stereomerically pure sibutramine-based compound or pharmaceutically acceptable salt, solvate, hydrate, ester, clathrate, or prodrug thereof is administered orally, mucosally, or transdermally. More preferably, it is administered transdermally.

Another method encompassed by this embodiment is a method of treating or preventing weight gain associated with smoking cessation which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof.

Another embodiment encompasses a method of treating or preventing weight gain associated with the administration of other drugs that may induce weight gain, which comprises administering to a patient in need of such treatment or prevention a therapeutically or prophylactically effective amount of a racemic or stereomerically pure sibutramine-based compound or a pharmaceutically acceptable salt, solvate, ester, clathrate, or prodrug thereof. One of skill in the art will recognize those at risk of or predisposed to weight gain and in need of prevention of such include, but are not limited to, individuals who, for example, are taking a drug or prescribed a drug that may induce weight gain.

Another embodiment encompasses a method of treating or preventing a chronic disorder including, but not limited to, narcolepsy, chronic fatigue syndrome, seasonal affective disorder, fibromyalgia, and premenstrual syndrome (or premenstrual dysphoric disorder), which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof. Examples of chronic disorders include, but are not limited to, narcolepsy, chronic fatigue syndrome, seasonal affective disorder, fibromyalgia, and premenstrual syndrome (or premenstrual dysphoric disorder), perimenopause, and menopause). Preferred methods are methods of treating or preventing narcolepsy, premenstrual syndrome, or chronic fatigue syndrome. One of skill in the art will recognize those at risk of or predisposed to chronic disorders and in need of prevention of such include, but are not limited to, individuals who, for example, have difficulty sleeping, suffer from depression, or irritability.

Another embodiment encompasses a method of treating or preventing anxiety, which comprises administering to a patient in need of such treatment or prevention a therapeutically or prophylactically effective amount of a racemic or stereomerically pure sibutramine-based compound or a pharmaceutically acceptable salt, solvate, hydrate, ester, clathrate, or prodrug thereof. One of skill in the art will recognize those at risk of or predisposed to anxiety and in need of prevention of such include, but are not limited to, individuals who, for example, are under high stress, exhibit sleeplessness or restlessness.

Another embodiment encompasses a method of treating or preventing an eating disorder including, but not limited to, anorexia, bulimia, binging, and snacking, which comprises administering to a patient in need of such treatment or prevention a therapeutically or prophylactically effective amount of a racemic or stereomerically pure sibutramine or a racemic or stereomerically pure sibutramine-based compound metabolite, or a pharmaceutically acceptable salt, solvate, hydrate, ester, clathrate, or prodrug thereof.

Another embodiment encompasses a method of treating or preventing migraines which comprises administering to a patient in need of such treatment or prevention a therapeutically or prophylactically effective amount of a racemic or stereomerically pure sibutramine-based compound, or a pharmaceutically acceptable salt, solvate, hydrate, ester, clathrate, or prodrug thereof. One of skill in the art will recognize those at risk of or predisposed to migraines and in need of prevention of such include, but are not limited to, individuals who, for example, suffer from depression, irritability, restlessness, or anorexia and may be associated with aura (i.e., transient, reversible, neurologic visual, somatosensory, motor, or language deficit).

Another embodiment encompasses a method of treating or preventing incontinence which comprises administering to a patient in need of such treatment or prevention a therapeutically or prophylactically effective amount of a racemic or stereomerically pure sibutramine-based compound or a pharmaceutically acceptable salt, solvate, ester, clathrate, or prodrug thereof. In particular embodiment, the racemic or stereomerically pure sibutramine-based compound can be used to treat fecal incontinence, stress urinary incontinence (“SUI”), urinary exertional incontinence, urge incontinence, reflex incontinence, passive incontinence, anal leakage, and overflow incontinence. In a particular embodiment, the method can treat or prevent incontinence in children (e.g., younger than 18) or in elderly (e.g., older 50) patients.

As used herein, the term “treating or preventing incontinence” means treatment, prevention of, or relief from the symptoms of incontinence including involuntary voiding of feces or urine, and dribbling or leakage or feces or urine, which may be due to one or more causes including, but not limited to, pathology altering sphincter control, loss of cognitive function, overdistention of the bladder, hyper-reflexia and/or involuntary urethral relaxation, weakness of the muscles associated with the bladder or neurologic abnormalities.

A preferred method encompassed by this embodiment is a method of treating or preventing stress urinary incontinence. In a further preferred method encompassed by this embodiment, the patient is an elder human of an age greater than 50 or a child of an age less than 13.

4.4. Combination Therapy

The invention also encompasses a method of treating or preventing male and female sexual function disorders, such as erectile dysfunction, which comprises adjunctively administering to a patient in need of such treatment or prevention a therapeutically or prophylactically effective amounts of a racemic and stereomerically pure sibutramine-based compound or pharmaceutically acceptable salts, solvates, hydrates, esters, clathrates, and prodrugs thereof in combination with a 5-HT₃ antagonist, a phosphodiesterase inhibitor, or a lipase inhibitor for obesity or weight management. Particularly preferred racemic and stereomerically pure sibutramine-based compounds are 1-hydroxyl, 3-hydroxy, or 7-hydroxy sibutramine-based compounds.

Preferred 5-HT₃ antagonists are antiemetic agents. Examples of suitable 5-HT₃ antagonists include, but are not limited to, granisetron (KYTRIL®), metoclopramide (REGLAN®), ondansetron (ZOFRAN®), renzapride, zacopride, tropisetron, and stereomerically pure stereoisomers, active metabolites, and pharmaceutically acceptable salts, solvates, hydrates, esters, clathrates, or prodrugs thereof.

Phosphodiesterase inhibitors that can be combined with compounds of the invention are disclosed in U.S. Pat. No. 5,250,534; U.S. Pat. No. 5,719,283; U.S. Pat. No. 6,127,363; WO 94/28902; WO 97/03675; WO 98/06722, each of which are expressly incorporated herein by reference in their entirety. Preferred phosphodiesterase inhibitors are PDE5 and PDE6 inhibitors. Particular phosphodiesterase inhibitors include, but are not limited to, sildenophil (Viagrae), desmethylsildenophil, vinopocetine, milrinone, amrinone, pimobendan, cilostamide, enoximone, peroximone, vesnarinone, rolipran, R020-1724, zaprinast, and dipyridamole.

The invention also encompasses a method of treating or preventing disorders associated with the administration of a lipase inhibitor for obesity or weight management which comprises adjunctively administering to a patient in need of such treatment or prevention a therapeutically or prophylactically effective amounts of a racemic and stereomerically pure sibutramine-based compound or pharmaceutically acceptable salts, solvates, hydrates, esters, clathrates, and prodrugs thereof in combination with a lipase inhibitor. A preferred lipase inhibitor for obesity or weight management includes, but is not limited to, orlistat (XENICAL®). Particularly preferred racemic and stereomerically pure sibutramine-based compound are 1-hydroxyl, 3-hydroxy, or 7-hydroxy sibutramine-based compounds.

In each of the methods of the invention, the racemic or stereomerically pure sibutramine-based compound, or a pharmaceutically acceptable salt, solvate, hydrate, ester, clathrate, or prodrug thereof, can adjunctively administered with one or more additional pharmacologically active compounds, e.g., the sibutramine-based compound and at least one additional pharmacologically active compound are administered as a combination, concurrently but separately, or sequentially by any suitable route (e.g., orally, transdermally, or mucosally).

In a preferred method of this embodiment, the racemic and stereomerically pure sibutramine-based compound is administered transdermally, orally, parenterally, or mucosally (e.g., nasally, sublingually, or buccally). In a more preferred method of this embodiment, the racemic and stereomerically pure sibutramine-based compound and the 5-HT₃ antagonist are both administered orally, transdermally, or mucosally. In another preferred method of this embodiment, the racemic and stereomerically pure sibutramine-based compound and the phosphodiesterase inhibitor are both administered orally, transdermally, or mucosally. In still another preferred method of this embodiment, the racemic and stereomerically pure sibutramine-based compound and the lipase inhibitor are both administered transdermally, orally, or mucosally.

Disorders that can be alleviated or prevented by adjunctively administering a racemic and stereomerically pure sibutramine-based compound or pharmaceutically acceptable salt, solvate, clathrate, hydrate, prodrug thereof with a lipase inhibitor for weight or obesity management include, but are not limited to, oily fecal spotting, flatus with discharge, fecal urgency, fatty/oily stool, oily evacuation, increased defecation, anal leakage, and fecal incontinence.

Another embodiment encompasses a racemic and stereomerically pure sibutramine-based compound and an additional pharmacologically active compound. Preferably the additional pharmacologically active compound is a selective serotonin reuptake inhibitors; 5-HT agonists and antagonists; phosphodiesterase inhibitors; hypnotics and sedatives; drugs useful in treating psychiatric disorders; CNS stimulants; dopamine receptor agonists; antimonic agents; lipase inhibitors for obesity and weight management; antipanic agents; cardiovascular agents; antivirals; antibiotics; antiffingals; or antineoplastics.

In another preferred embodiment, the pharmaceutical compositions and dosage forms comprise an additional pharmacologically active compound. In a preferred embodiment, the additional pharmacologically active compound is a drug that affects the central nervous system is a 5-HT agonists and antagonist; hypnotics and sedatives; drugs useful in treating psychiatric disorders; CNS stimulants; dopamine receptor agonists; antimonic agents; antipanic agents; cardiovascular agents; antivirals; antibiotics; antifungals; or antineoplastics. In a particular embodiment, the 5-HT₃ antagonist is an antiemetic agent.

In still another preferred embodiment, the pharmaceutical compositions and dosage forms comprise a 5-HT₃ antagonist that is granisetron, metoclopramide, ondansetron, renzapride, zacopride, tropisetron, stereomerically pure stereoisomers, active metabolites thereof, and pharmaceutically acceptable salts, solvates, hydrates, esters, clathrates, and prodrugs thereof. In a preferred embodiment, the amount of 5-HT₃ antagonist is from about 0.5 mg to about 500 mg, from about 1 mg to about 350 mg, from about 2 mg to about 250 mg.

In still another preferred embodiment, the pharmaceutical compositions and dosage forms comprise a phosphodiesterase inhibitor including, but are not limited to, PDE5 and PDE6 inhibitors, sildenophil (Viagra®), desmethylsildenophil, vinopocetine, milrinone, amrinone, pimobendan, cilostamide, enoximone, peroximone, vesnarinone, rolipram, R020-1724, zaprinast, and dipyridamole. In a preferred embodiment, the amount of phosphodiesterase inhibitor is from about 0.5 mg to about 500 mg, from about 1 mg to about 350 mg, from about 2 mg to about 250 mg.

Additional pharmacologically active compounds that can be used in the methods and compositions of the invention include, but are not limited to, drugs that act on the central nervous system (“CNS”), such as, but not limited to: 5-HT (e.g., 5-HT₃ and 5-HT_{1A}) agonists and antagonists; selective serotonin reuptake inhibitors (“SSRIs”); hypnotics and sedatives; drugs useful in treating psychiatric disorders including antipsychotic and neuroleptic drugs, antianxiety drugs, antidepressants, and mood-stabilizers; CNS stimulants such as amphetamines; dopamine receptor agonists; antimonic agents; antipanic agents; cardiovascular agents (e.g., beta blockers and angiotensin converting enzyme inhibitors); antivirals; antibiotics; antifungals; and antineoplastics.

More specific drugs that act on the CNS include, but are not limited to, SSRIs, benzodiazepine compounds, tricyclic antidepressants, antipsychotic agents, anti-anxiolytic agents, β-adrenergic antagonists, 5-HT_{1A} receptor antagonists, and 5-HT₃ receptor agonists. Even more specific drugs that act on the CNS include, but are not limited to, lorazepam, tomoxetine, olanzapine, respiradone, buspirone, hydroxyzine, and valium.

Selective serotonin reuptake inhibitors are compounds that inhibit the central nervous system uptake of serotonin while having reduced or limited affinity for other neurologically active receptors. Examples of SSRIs include, but are not limited to, citalopram (CELEXA®); fluoxetine (PROZAC®) fluvoxamine (LUVOX®); paroxetine (PAXIL®); sertraline (ZOLOFT®); venlafaxine (EFFEXOR®); and stereomerically pure stereoisomers, active metabolites, and pharmaceutically acceptable salts, solvates, hydrates, esters, clathrates, and prodrugs thereof.

Benzodiazepine compounds that can be used in the methods and compositions of the invention include, but are not limited to, those described in Goodman & Gilman, The Pharmacological Basis of Therapeutics, 362-373 (9^{th} ed. McGraw-Hill, 1996). Examples of specific benzodiazepines include, but are not limited to, alprazolamn, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepaam, triazolam, pharmacologically active metabolites and stereoisomers thereof, and pharmaceutically acceptable salts, solvates, hydrates, esters, clathrates, and prodrugs thereof. The tradenames of some of these compounds are provided below.

The clinician, physician, or psychiatrist will appreciate which of the above compounds can be used in combination with a racemic or stereomerically pure sibutramine-based compound or a pharmaceutically acceptable salt, solvate, hydrate, ester, clathrate, or prodrug thereof, for the treatment or prevention of a given disorder, although preferred combinations are disclosed herein.

Disorders that can be treated or prevented using a racemic or stereomerically pure sibutramine metabolite, or a pharmaceutically acceptable salt, solvate, hydrate, clathrate, or prodrug thereof, in combination with a benzodiazepine such as those listed above include, but are not limited to, depression, affective disorders, anxiety, eating disorders, and cerebral function disorders such as those described herein.

The invention further encompasses methods of using and pharmaceutical compositions comprising racemic or stereomerically pure sibutramine-based compound or a pharmaceutically acceptable salt, solvate, hydrate, ester, clathrate, or prodrug thereof, in combination with an antipsychotic agent. Antipsychotic agents are used primarily in the management of patients with psychotic or other serious psychiatric illness marked by agitation and impaired reasoning. These drugs have other properties that possibly are useful clinically, including antiemetic and antihistamine effects and the ability to potentiate analgesics, sedatives, and general anesthetics. Specific antipsychotic drugs are tricyclic antipsychotic drugs, of which there are three subtypes: phenothiazines, thioxanthenes, and other heterocyclic compounds, all of which can be used in the methods and compositions of the invention. See, e.g., Goodman & Gilman, The Pharmacological Basis of Therapeutics, 404 (9^{th} ed. McGraw-Hill, 1996).

Specific tricyclic antipsychotic compounds include, but are not limited to, chlorpromazine, mesoridazine, thioridazine, acetophenazine, fluphenazine, perphenazine, trifluoperazine, chlorprothixene, thiothixene, clozapine, haloperidol, loxapine, molindone, pimozide, risperidone, desipramine, pharmacologically active metabolites and stereoisomers thereof, and pharmaceutically acceptable salts, solvates, hydrates, esters, clathrates, and prodrugs thereof. The tradenames of some of these compounds are provided herein.

Disorders that can be treated or prevented using racemic or stereomerically pure sibutramine-based compounds or a pharmaceutically acceptable salt, solvate, hydrate, ester, clathrate, or prodrug thereof, in combination with an antipsychotic compound, and particularly a tricyclic antipsychotic compound, include, but are not limited to, affective disorders (e.g., depression), anxiety, eating disorders, and cerebral function disorders (e.g., schizophrenia) such as those described herein.

The invention further encompasses methods of using and pharmaceutical compositions comprising a racemic or stereomerically pure sibutramine-based compound or a pharmaceutically acceptable salt, solvate, hydrate, ester, clathrate, or prodrug thereof, in combination with a 5-HT_{1A} receptor antagonist and/or a β-adrenergic antagonist. Examples of 5-HT_{1A} receptor antagonists and B-adrenergic antagonists that can be used in the methods and compositions of the invention include, but are limited to: alprenolol; WAY 100135; spiperone; pindolol; (S)-UH-301; penbutolol; propranolol; tertatolol; a compound of the formula I as disclosed in U.S. Pat. No. 5,552,429, which is incorporated herein by reference; pharmacologically active metabolites and stereoisomers thereof; and pharmaceutically acceptable salts, solvates, hydrates, esters, clathrates, and prodrugs thereof.

Disorders that can be treated or prevented using racemic or stereomerically pure sibutramine-based compound or a pharmaceutically acceptable salt, solvate, hydrate, ester, clathrate, or prodrug thereof, in combination with a 5-HT_{1A} receptor antagonist include, but are not limited to, depression, obsessive-compulsive disorders, eating disorders, hypertension, migraine, essential tremor, hypertrophic subaortic stenosis and pheochromocytoma. A specific disorder that can be treated or prevented is posttraumatic depression disorder.

Disorders that can be treated or prevented using racemic or stereomerically pure sibutramine-based compound or a racemic or stereomerically pure sibutramine metabolite, or a pharmaceutically acceptable salt, solvate, or clathrate thereof, in combination with a β-adrenergic antagonist include, but are not limited to, post myocardial infarction depression. Specific β-adrenergic antagonists include, but are not limited to, S(−)-pindolol, penbutolol, and propranolol.

The invention further encompasses methods of using and pharmaceutical compositions comprising racemic or stereomerically pure sibutramine-based compound or a pharmaceutically acceptable salt, solvate, hydrate, ester, clathrate, or prodrug thereof, in combination with a non-benzodiazepine or non-tricyclic agents. Examples of such additional pharmacologically active compounds include, but are limited to: olanzapine, buspirone, hydroxyzine, tomoxetine, pharmacologically active metabolites and stereoisomers thereof, and pharmaceutically acceptable salts, solvates, hydrates, esters, clathrates, and prodrugs thereof.

Disorders that can be treated or prevented using racemic or stereomerically pure sibutramine-based compound or a pharmaceutically acceptable salt, solvate, or clathrate thereof, in combination with a compound include, but are not limited to, lorazepam, tomoxetine, olanzapine, respiradone, buspirone, hydroxyzine, valium, pharmacologically active metabolites and stereoisomers thereof, and pharmaceutically acceptable salts, solvates, hydrates, esters, clathrates, and prodrugs thereof include, but are not limited to, anxiety, depression, hypertension, and attention deficit disorders.

While all combinations of racemic or stereomerically pure sibutramine-based compounds or pharmaceutically acceptable salts, solvates, hydrates, esters, clathrates, and prodrugs thereof, and one or more above described pharmacologically active compounds can be useful and valuable, certain combinations are particularly preferred. Examples of preferred combinations include those wherein a racemic or stereomerically pure sibutramine metabolite, or a pharmaceutically acceptable salt, solvate, or clathrate thereof, is combined with one of the following:

| | | |
|---|---|---|
| alprazolam; | thioridazine; | desipramine; |
| brotizolam; | acetophenazine; | clonidine; |
| chlordiazepoxide; | fluphenazine; | olanzapine; |
| clobazam; | perphenazine; | methylphenidate; |
| clonazepam; | trifluoperazine; | buspirone; |
| clorazepate; | chlorprothixene; | hydroxyzine, |
| demoxepam; | thiothixene; | tomoxetine. |
| diazepam; | clozapine; | sildenophil; |
| estazolam; | haloperidol; | desmethylsildenophil |
| flumazenil; | loxapine; | vinopocetine; |
| flurazepam; | molindone; | mirinone; |
| halazepam; | pimozide; | amrinone; |
| lorazepam; | risperidone; | pimobendan; |
| midazolam; | alprenolol; | cilostamide; |
| nitrazepam; | WAY 100135; | enoximone; |
| nordazepam; | spiperone; | peroximone; |
| oxazepam; | S(−)-pindolol; | vesnarimone; |
| prazepam; | R(+)-pindolol; | rolipran; |
| quazepam; | racemic pindolol; | R020-1724; |
| temazepam; | (S)-UH-301; | zaprinast; and |
| triazolam; | penbutolol; | dipyridamole. |
| chlorpromazine; | propranolol; | |
| mesoridazine; | tertatolol; | |

In one embodiment, pharmaceutical compositions and dosage forms of the invention comprise a dopamine reuptake inhibitor, such as racemic or stereomerically pure sibutramine-based compounds or a pharmaceutically acceptable salt, solvate, hydrate, ester, clathrate, or prodrug thereof, and optionally an additional pharmacologically active compound, such as a 5-HT₃ antagonist. The pharmaceutical compositions and dosage forms can contain a pharmaceutically acceptable carrier and optionally other therapeutic ingredients known to those skilled in the art.

Suitable daily dosage ranges of additional pharmacologically active compounds that can be adjunctively administered with a racemic or stereomerically pure sibutramine-based compound can be readily determined by those skilled in the art following dosages reported in the literature and recommended in the Physician 's Desk Reference® (54^{th} ed., 2000).

For example, suitable daily dosage ranges of 5-HT₃ antagonists can be readily determined by those skilled in the art and will vary depending on factors such as those described above and the particular 5-HT₃ antagonists used. In general, the total daily dose of a 5-HT₃ antagonist for the treatment or prevention of a disorder described herein is from about 0.5 mg to about 500 mg, preferably from about 1 mg to about 350 mg, and more preferably from about 2 mg to about 250 mg per day.

The therapeutic or prophylactic administration of an active ingredient of the invention is preferably initiated at a lower dose, e.g., from about 0.01 mg to about 1 mg of racemic or stereomerically pure sibutramine-based compound and optionally from about 15 mg to about 60 mg of 5-HT₃ antagonist, and increased, if necessary, up to the recommended daily dose as either a single dose or as divided doses, depending on the global response of the patient. It is further recommended that patients aged over 65 years should receive doses of racemic or stereomerically pure sibutramine-based compound in the range of from about 0.01 mg to about 10 mg per day depending on global response. It may be necessary to use dosages outside these ranges, which will be readily determinable by one of ordinary skill in the pharmaceutical art.

Adjunctively administering of two or more active ingredients in accordance with the methods of the invention can be concurrent, sequential, or both. For example, a dopamine reuptake inhibitor and a 5-HT₃ antagonist can be administered as a combination, concurrently but separately, or by sequential administration.

4.5. Synthesis

Sibutramine and its potential metabolites can be depicted by the following scheme:

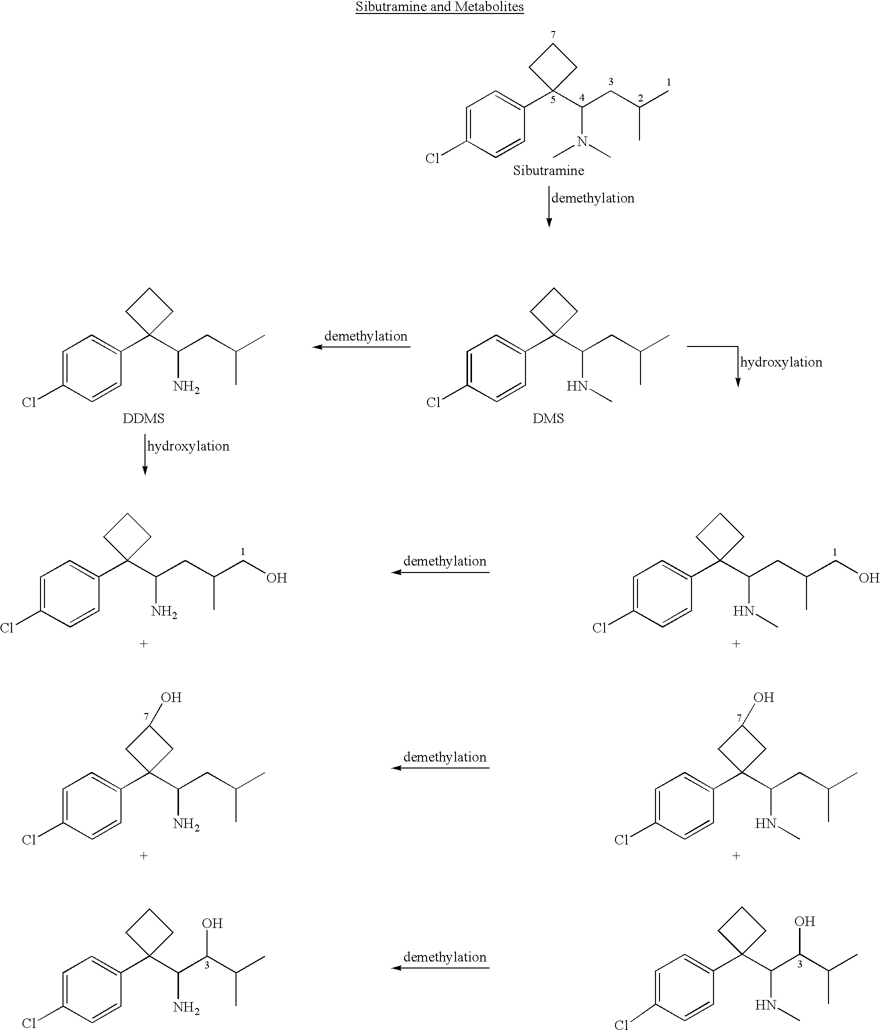

As discussed below, this invention encompasses a methods of preparing each of the metabolites of sibutramine, as well as stereomerically pure forms, derivatives, salts, solvates, clathrates, and prodrugs thereof.

4.5.1. Synthesis of 1-Hydroxy Derivatives of Sibutramine

The synthesis of the 1-hydroxyalted derivatives of sibutramine metabolites involves the diastereoselective addition of an organometallic reagent to a common synthon, e.g., (R)-tert-butylsulfinimide (Scheme 12). See Liu, G., et al., J. Am. Chem. Soc. 119:9913-9914 (1997). This method provides access to all four 1-hydroxyl didesmethyl isomers, enantioselectively and in high yield by proper choice of reaction conditions. These can easily then be converted to stereomerically pure 1-hydroxyl-desmethyl isomers by N-methylation and further N-methylated to 1-hydroxyl sibutramine.

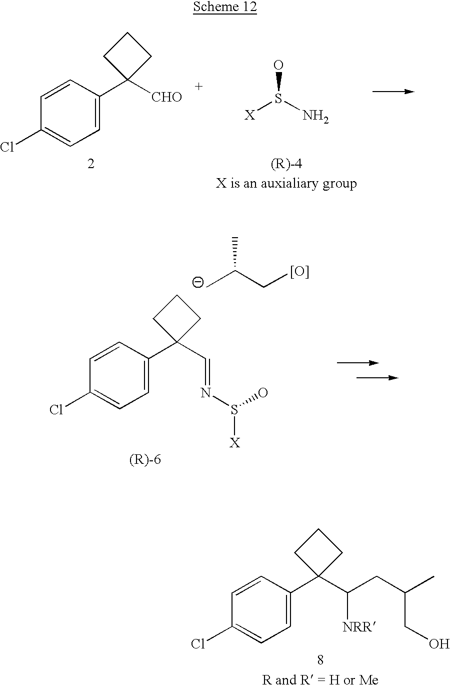

As used herein and unless otherwise stated, the term “auxiliary group” refers to any group that is used to induce asymmetry in a reaction or influence the addition of a substrate across a double bond and then removed. Examples of auxiliaries include, but are not limited to, phenyl, tolyl, naphthyl, and tert-butyl. One of skill in the art will recognize that auxiliaries used in compounds of the invention can be removed and replaced by a different auxiliary. One of skill in the art will also recognize that some auxiliaries induce greater asymmetry or have a greater influence over addition across a double bond then others.

The four hydroxyl DDMS iomers have been made as free bases and as the corresponding (D)-tartrate salt in stereomerically pure form, and have been used for biological testing (Scheme 13). The stereochemistry at C-2 was determined by the X-ray crystallography.

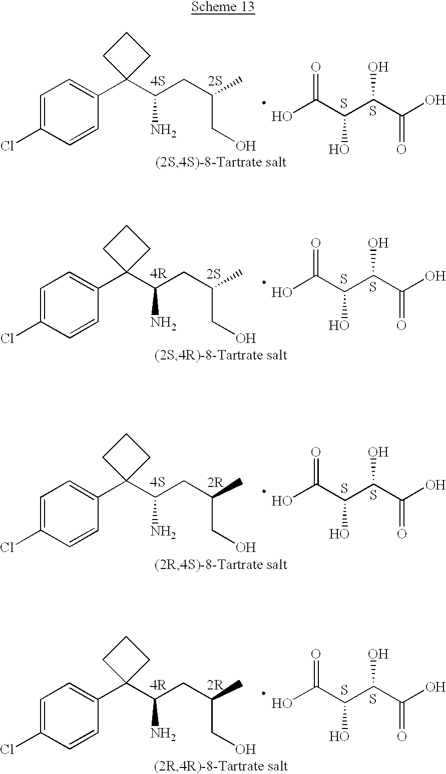

The synthesis of the hydroxylated DMS isomers employs a similar approach, as in preparing the free-base of hydroxylated DDMS, which are converted into the desired compounds by a sequence of formylation and reduction (Scheme 14). For example, one preparation sequence starts with the condensation of (R)-tert-butylsulfinamide 4 with aldehyde 2 in THF, catalyzed by Ti(OEt)₄ to provide the sulfinimine (R)-6. Addition of the Grignard reagent 5 to R-6 in CH₂Cl₂ proceeded at room temperature to give (2S,4S)-7 and (2S,4R)-7′ in a ratio of 96.4:3.6. Separation of the major isomer by column chromatography, followed by cleavage of the chiral auxiliary, afforded the primary amine (2S,4S)-8. N-Methylation of (2S,4S)-8 was achieved by heating with formic acid in toluene at 100° C., followed by reduction of formamide using borane at room temperature to produce the secondary amine (2S,4S)-N-Me-8. Treatment of 8 with D-tartaric acid in methanol formed the corresponding (2S,4S)-N-Me-8 tartrate salt as a white solid. Treatment of N-Me-8 free base a second time with a methylating agent afforded the (2S,4S)-N,N-Me₂-8.

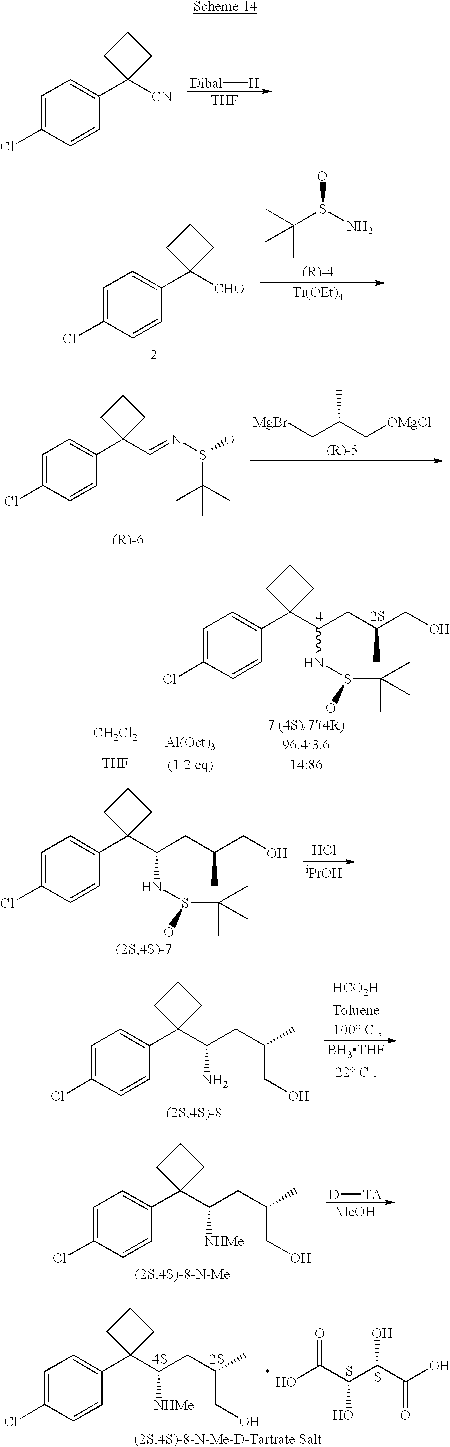

The diastereomer was prepared by the same sequence, except the addition of the Grignard reagent 5 to (R)-6 was carried out in THF in the presence of 1.2 eq of Al(Oct)₃, giving 14:86 ratio of (2S,4S)-7 and (2S,4R)-7′. It is worthy to note that the formation of salt in this case has to be treated with HCl in ethanol, to afford the corresponding HCl ethanol solvate (Scheme 15).

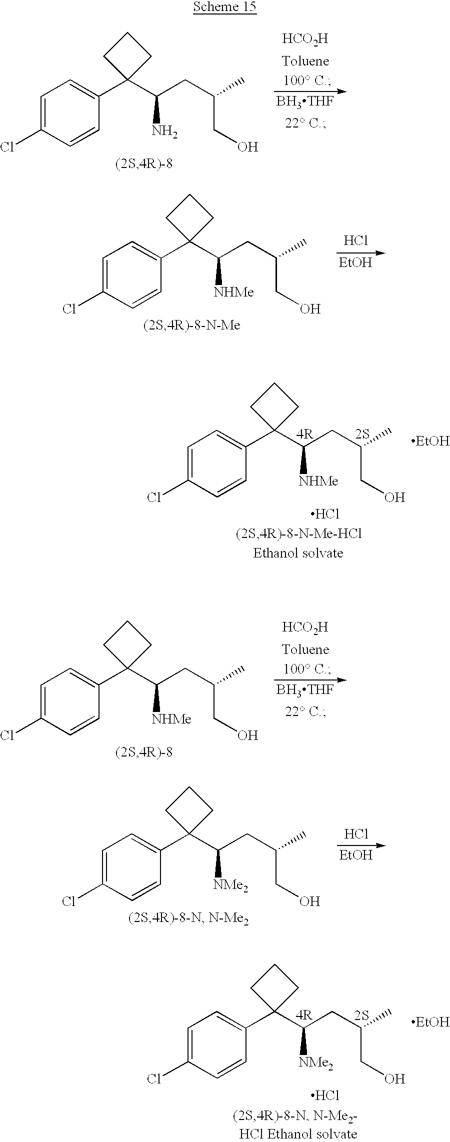

Similiary, the other two diastereomers (2R,4S)-5 and (2R,4R)-5 were prepared by addition of (S)-5 to 6 in dichloromethane catalyzed by 1.2 eq of Al(Oct)₃, and in THF in the absence of Al(Oct)₃, respectively (Scheme 16). In the former case, the addition delivered the diastereomers (2R,4S)-7 and (2R,4R)-7′ in the ratio of 95:5, while in the latter case, the reaction afforded the diastereomeric mixture in the ratio of 15:85, favoring the formation of the 4R isomer. Conversion of (2R,4S)-7 and (2R,4R)-7′ into the corresponding sibutramine metabolites was achieved by the same sequence as described above. Exposure of the (2R,4S)-8-N-Me free base to HCl in ethanol at room temperature gave the corresponding HCl salt as ethanol solvate. Alternatively, the salt formation of (2R,4S)-8-N-Me was achieved by treatment with L-tartaric acid in methanol, to provide the corresponding tartrate.

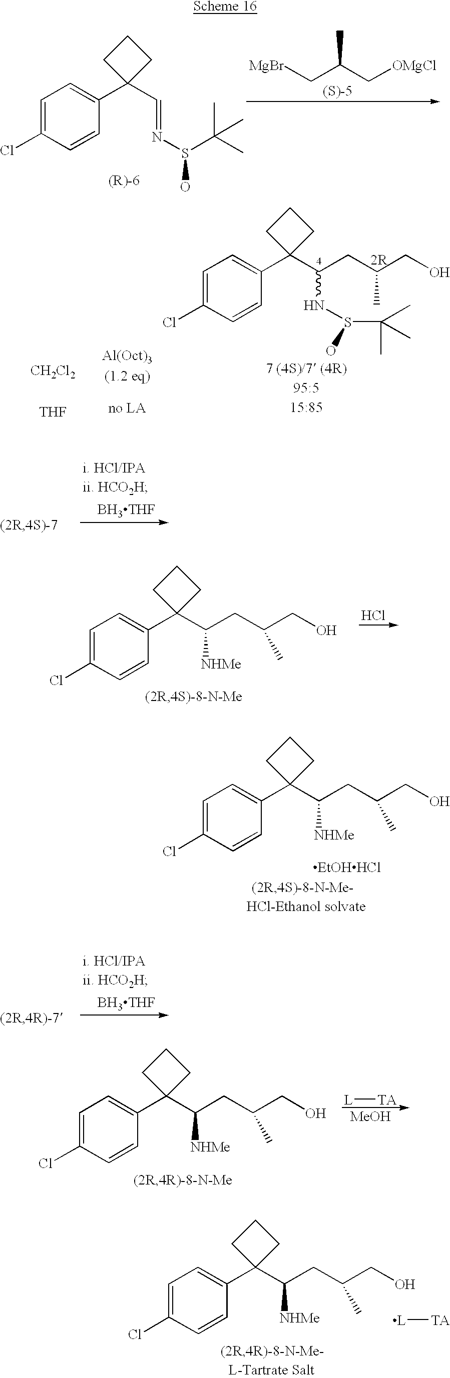

Alternatively, the diasteromers (2R,4R)-8 and (2S,4S)-8 could be prepared via a highly diastereoselective reduction of the imine intermediate 10 by taking advantage of the pre-existing chiral center at C-2. For example, addition of Grignard reagent (S)-5 to 1-(4-Chloro-phenyl)-cyclobutanecarbonitrile (CCBC) in ethyl ether gave the imine intermediate 10 which was reduced efficiently by novel reducing agents such as [1,3,2]Dioxaborepane-4,7-dione (12) or Benzo[1,3,2]dioxaborinin-4-one (14). A mixture of diastereomers (2R,4R)-8 and (2S,4S)-8 was obtained in the ratio of up to 95:5, favoring the formation of (2R,4R)-8. Reducing agent 12 or 14 were generated by treatment of BH₃.THF with succinic acid or salicylic acid, respectively (Scheme 17).

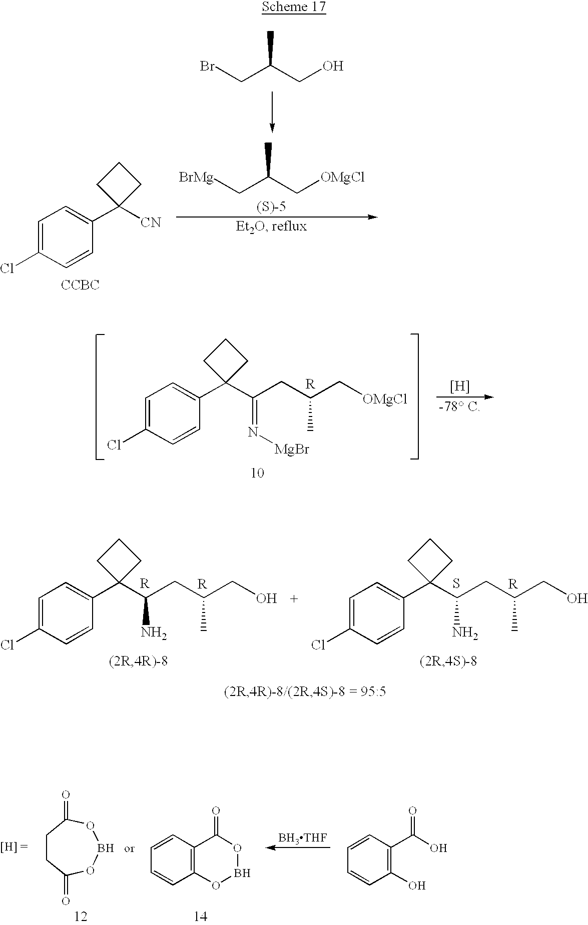

Because 1-hydroxylated desmethylsibutramine and didesmethylsibutramine are basic amines, diastereomeric salts of these compounds that are suitable for separation by fractional crystallization are readily formed by addition of stereomerically pure chiral acid resolving agents. Suitable resolving agents include, but are not limited to, stereomerically pure tartaric, camphorsulfonic acid, mandelic acid, and derivatives thereof. Stereomerically pure isomers of sibutramine, desmethylsibutramine, and didesmethylsibutramine can be recovered either from the crystallized diastereomer or from the mother liquor, depending on the solubility properties of the particular acid resolving agent employed and the particular acid enantiomer used. The identity and optical purity of the particular sibutramine-based compound or isomer so recovered can be determined by polarimetry or other analytical methods.

4.5.2. Synthesis of 3-Hydroxyl Sibutramine Derivatives

The synthesis of the 3-hydroxyl sibutramine derivatives can be synthesized is various ways. One synthesis utilizes a pair of enantiomers (S)-6 and (R)-6, which were obtained by condensation of aldehyde 2 with (S)- and (R)-tert-butylsulfinamide 4, respectively (Scheme 18).

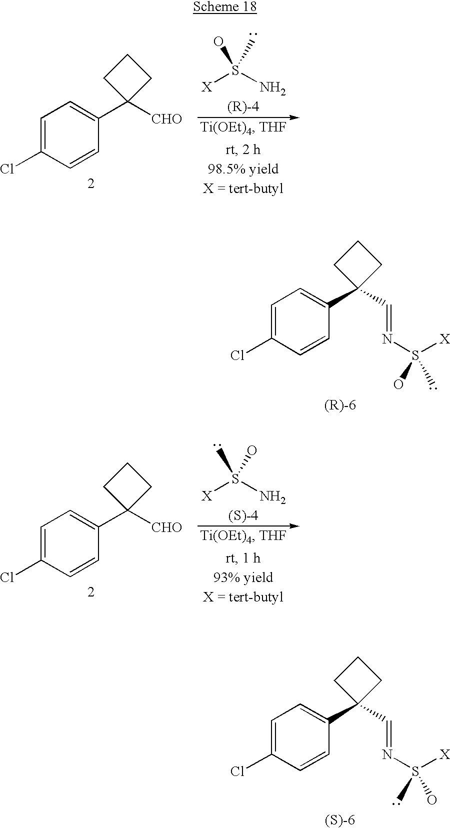

Addition of 2 equivalents of racemic (∝-methoxymethoxy) isobutyl lithium 18, which was readily prepared in situ from the exchange reaction of (α-methoxymethoxy) isobutyl tri-n-butylstannane 16 with n-butyllithium in THF at −78° C., to sulfinimine (R)-6 produced in 79% isolated yield, a mixture of diastereomers (3R,4S)-20 and (3S,4S)-20, in the ratio of 72:28, favoring the formation of (3R,4S)-20. Cleavage of protecting group by refluxing in methanolic HCl gave the unseparable mixture of hte aminoalcohol (3R,4S)-24 and (3S,4S)-24 as the corresponding HCl salt. Cyclization of the mixture (3R,4S)-24 and (3S,4S)-24, by treatment with carbonyldiimidazole and Et₃N in CH₂Cl₂, afforded a separable mixture of (3R,4S)-22 and (3S,4S)-22′ in 73 percent yield. Hydrolysis of the separated carbamate (3R,4S)-22 or (3S,4S)-22 with potassium hydroxide in ethylene glycol, in the presence of catalytic amount of NH₂ NH₂, and subsequently treatment of the free aminoalcohols with methanolic HCl, obtained in 84 percent overall yield for 2 steps, in corresponding HCl salt of (3R,4S)-24 and (3S,4S)-24, respectively.

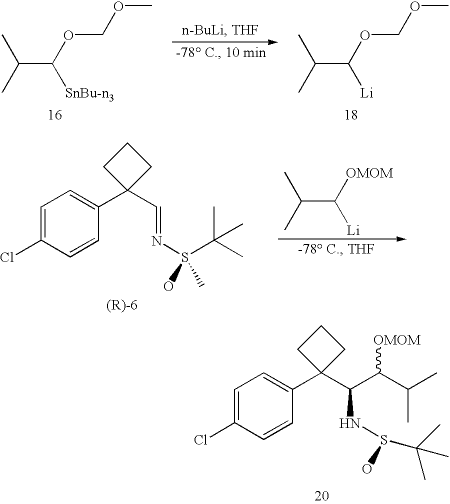

TABLE 1
Results of the reaction of (alpha-methoxymethoxy)isobutyl
lithium with aldimine 6.

| | Ratio of | | | | |
|---|---|---|---|---|---|
| Entry | Imine:lithium | Temp (° C.) | Time (h) | 13 (%) | anti:syn |
| 1 | 1:2 | −78 | 3 | 100 (90) | 72:28 |
| 2 | 1:1.1 | −78 | 2 | 100 (93) | 60:40 |
| 3 | 1:5 | −78 | 0.5 | 100 | 84:16 |
| 4 | 1:10 | −78 | 0.2 | 100 | 84:16 |
| 5* | 1:5 | −78-rt | 27 | <20 | — |

*The litium reagent was prepared in situ and mixed with 5 eq. MgBr₂-OEt₂.

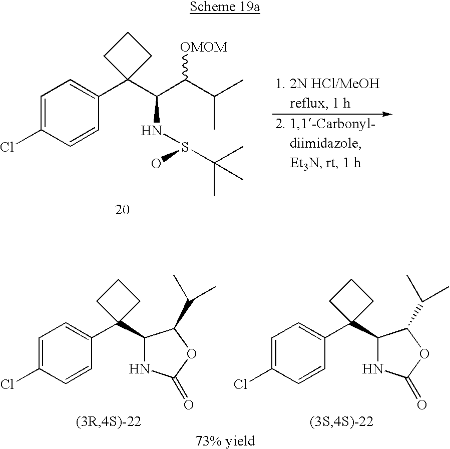

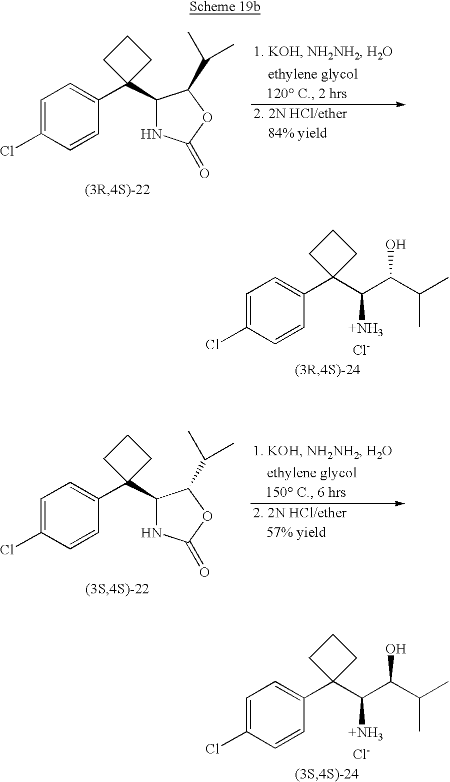

Alternatively, the two chiral centers of these aminoalcohols could be created by addition of stereomerically pure (α-methoxymethoxy) isobutyl lithium (18) to aldimine (R)- or (S)-6. Therefore, treatment of organolithium (S)-18, derived from the corresponding stannane (R)-16 (>95% ee), with aldimine (S)-6 in THF at −78° C., afforded exclusively (3S,4R)-20 in 92% isolated yield (Scheme 20). In this case, only less than 1% of (3S,4S)-20 was observed. Cleavage of the protecting group by refluxing in 2N HCl aqueous solution, provided the free aminoalcohol, which was then converted into the corresponding HCl salt (3S,4R)-24 by treatment dry methanolic HCl.

Similiarly, addition of organolithium reagent (R)-18, derived from the corresponding stannane (S)-16, to (S)-6 in THF at −78° C., gave a mixture of diastereomers (3R,4R)-20 and (3R,4S)-20 in the ratio of 99:1, with (3R,4R)-20 as the major product. (3R,4R)-20 was isolated in 61% yield and then converted into (3R,4R)-24 by the same sequence as described above for (3S,4R)-24.

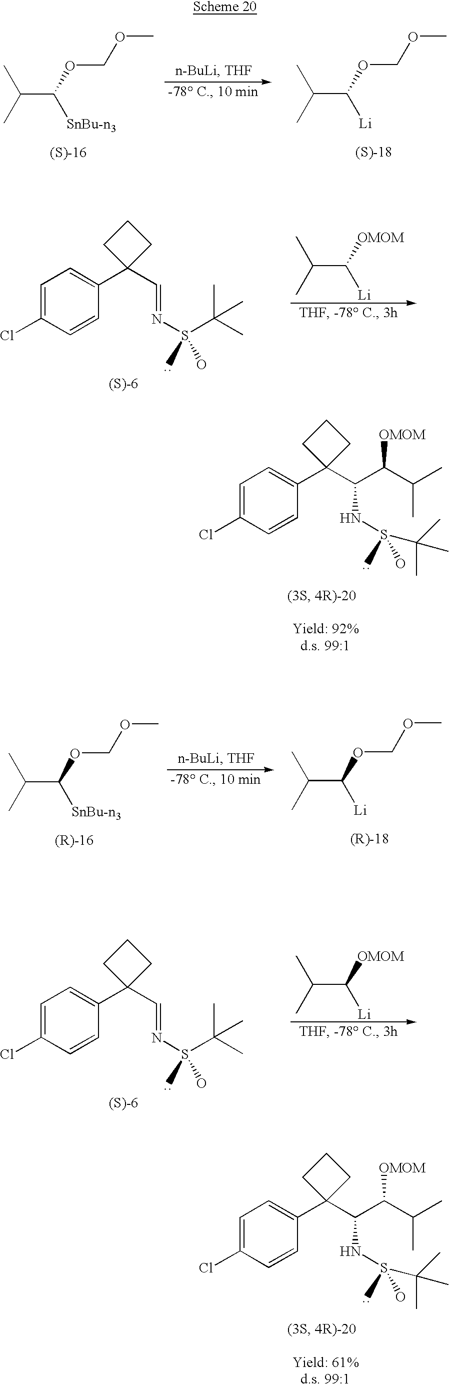

4.5.3. Synthesis of 7-Hydroxy Sibutramine Metabolite Derivatives

The asymmetric syntheses of 7-hydroxyl sibutramine-based compounds involves formation of the chiral center by the addition of an organometallic reagent, such as isobutyl lithium to (R)- or (S)-tert-butylsulfinyl imine 7, which could be derived from the condensation of (R)- or (S)-tert-butylsulfinyl amide with the corresponding cis and trans hydroxyl aldehyde 28. The structure of the cis and trans isomers 7-hydroxyl desmethylsibutramine and 7-hydroxyl didesmethylsibutramine are set forth below:

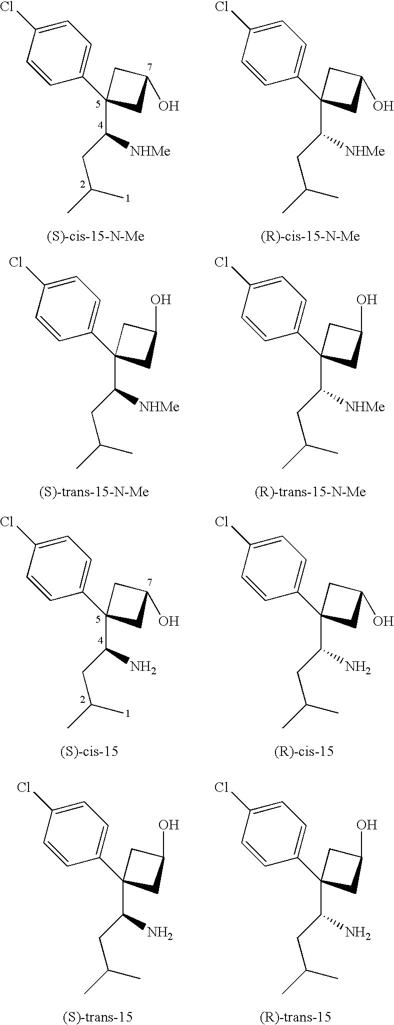

For example, the chiral center of (R)-configuration was created by the addition of an organolithium reagent to the (R)-tert-butylsulfinimide, prepared from the condensation of (R)-tert-butylsulfinyl amide 6 with the corresponding hydroxyl aldehyde 28 (Scheme 21).

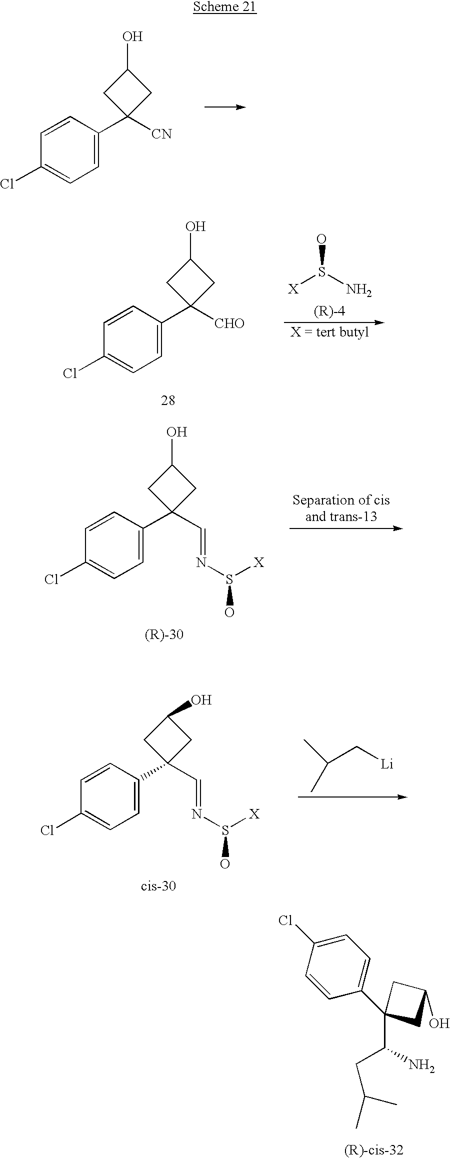

It was reported that the hydroxyl nitrile could be prepared from 4-chlorophenylacetonitrile [See Jeffrey et al, J. Chem. Soc. Perkin Trans 1, 1996, 2583] (Scheme 22). The route involves deprotonation with methyllithium in THF at -78° C., followed by treatment with epibromohydrin and methylmagnesium iodide. This approach gives hydroxylnitrile 34 as an epimeric mixture, in a ratio of approximately 2.6:1, favoring the cis epimer.

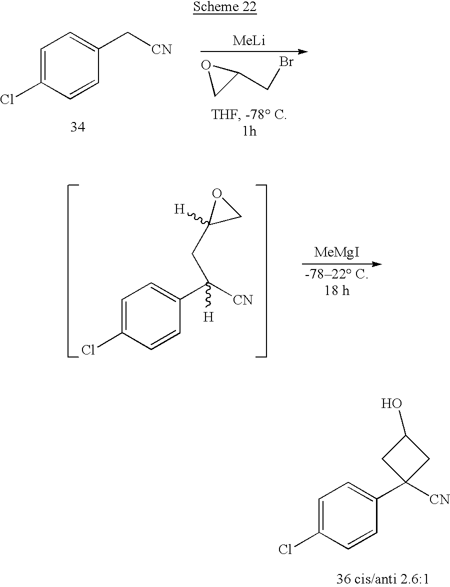

Conversion of hydroxyl nitrile mixture 36 to the corresponding cis/trans aldehydes 28 was achieved successfully in an 83% yield by using 2.2 equiv of Dibal-H (1.0 M in hexane) in THF at 0° C. Condensation of the hydroxyl aldehydes 28 (cis/trans 2.6 :1) with one equivalent of (R)-tert-butylsulfinyl amide 6 in the presence of Ti(OEt)₄ in THF at 22° C. for 10 h was incomplete, and provided a mixture of the cis and trans aldimines with the trans isomer as the major product. In this case, only the cis-aldehyde was recovered, indicating that there was a kinetic resolution effect for the formation of sulfinimines 30. It is possible to achieve the separation of isomers at this step by controlling the formation of the trans sulfinimine kinetically.

The condensation reaction was completed in a 91% isolated yield by heating the mixture in toluene to 100° C. for 1 h. A careful and difficult separation of the aldimines by column chromatography gave the corresponding two isomers trans- and cis-30 (Scheme 23).

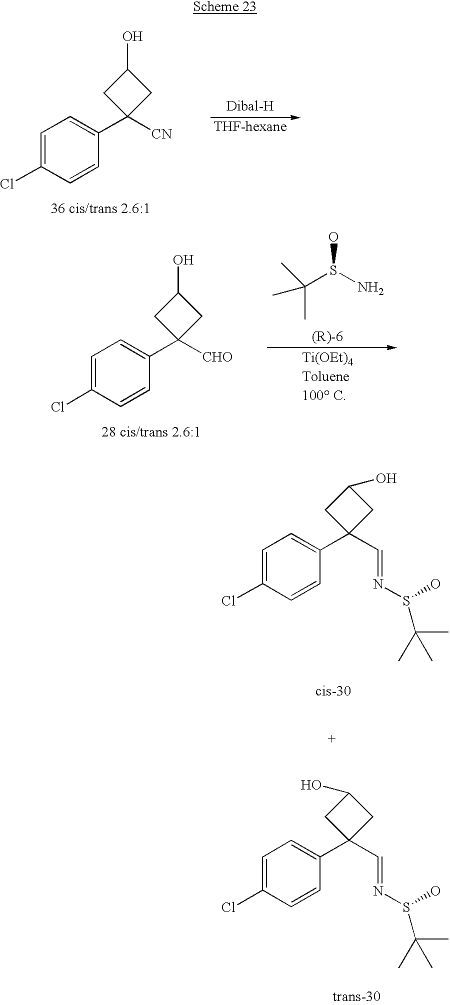

Addition of iso-butyllithium (3.2 eq) to cis-30 in TBF at −78° C. proceeded smoothly (Scheme 23). The reaction completed in 2 h, providing in a nearly quantitative yield, a mixture of two diastereomers in the ratio of 3:97, presumably in favor of cis-38′. Other conditions were investigated, including the effect of Lewis acid. The results are summarized in Table 2. It was found that the addition of 2.2 equiv of BF₃.Et₂O could further improve the diastereoselectivity, and give a mixture of cis-38 and cis-38′ in the ratio of 2:98 (entry 4, 5). The reaction was much slower in toluene, with only 45% of the products formed after 8 h at −78° C. In this case, the diastereoselectivity was also low (cis-38′/cis-38 48:52).

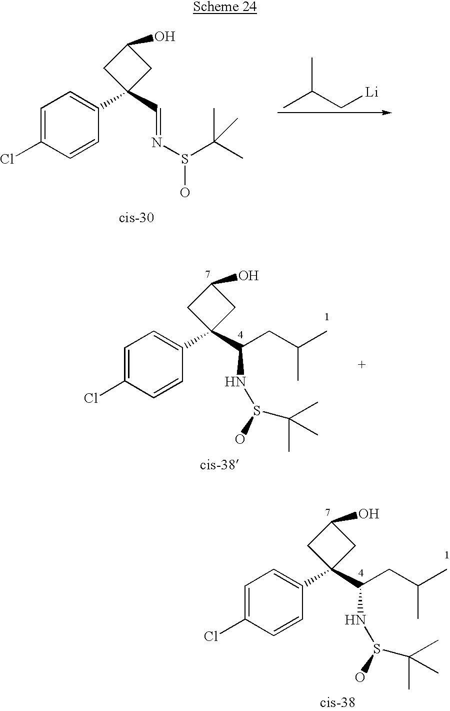

TABLE 2
Addition of i-BuLi to cis-30

| Entry | i-BuLi(eq.) | LA(eq.) | Solvent | Temp.(° C.) | Time (h) | d.r.(cis-38′/cis-38)Conv.(%) | |
|---|---|---|---|---|---|---|---|
| 1 | 3.2 | — | THF | −78 | 2 | 97:3 | 100 |
| 2 | 3.2 | | Toluene | −78 | 8 | 48:52 | 50 |
| 3 | 3.2 | | Et₂O | −78 | | | |
| 4 | 3.2 | BF₃Et₂O(2.2) | THf | −78 | 2 | 98:2 | 100 |
| 5 | 3.2 | BF₃Et₂O(3.2) | THE | −78 | 2 | 98.4:1.6 | 100 |
| 6 | 3.2 | A1(Oct)₃(2.5) | THE | −78 | 2 | 99:1 | 86 |

The addition of iso-butyllithium (3.2 eq.) to trans-30 in THF at −78° C. was completed in 2 h, producing a mixture of two diastereomers, trans-38 and trans-38′, in a ratio of 89:11, presumably in favor of trans-14. The product was isolated in a 74% yield, and the ratio of the isomer increased to 97:3 after chromatography (Scheme 25).

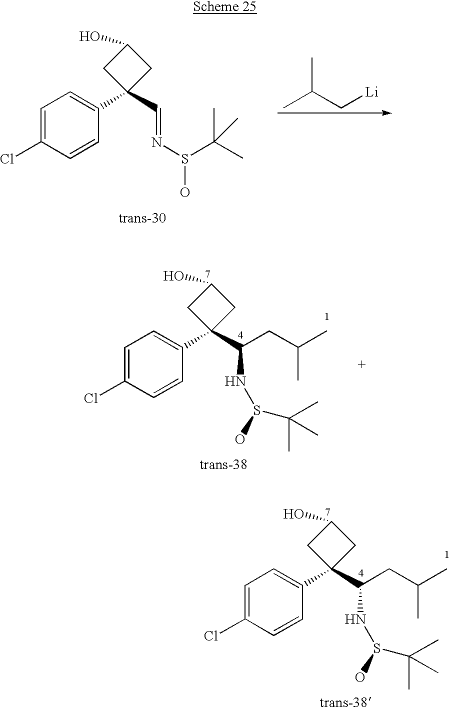

Deprotection then affords the desired 7-hydroxy didesmethylsibutramine metabolites. Methylation using conditions known to those skilled in the art then gives the 7-hydroxy desmethylsibutramine metabolites and a second methylation can afford 7-hydroxy sibutramine.

In summary, the invention includes the synthesis of an asymmetric route to two isomers, involving the separation of the cis- and trans-tert-butyl-sulfinimides 13, followed by a highly diastereoselective addition of iso-butyllithium to cis- and trans-13, respectively. Similiarly, by using the corresponding (S)-tert-butylsulfinimides, another two isomers could be obtained via the same sequence.

The invention is further defined by reference to the following examples. It will be apparent to those skilled in the art that many modifications, both to materials and methods, can be practiced without departing from the scope of this invention. It should also be noted that names of compounds below may differ from those set forth above in backbone numbering and positional numbering due to use of International Union of Pure and Applied Chemistry (IUPAC) naming below. Wherever a discrepancy between the depicted structure and the name of the compound, the depicted structure will take preference.

### 5. EXAMPLES

5.1. Synthesis of Hydroxylated Sibutramine Metabolites

5.1.1. 1-Hydroxyl Experimental Data

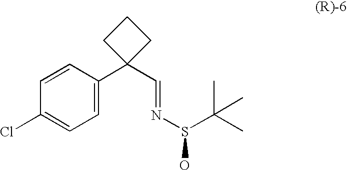

(R)-N-(1-(4-Chlorophenyl)cyclobutanemethylene)-t-butanesulfinamide:

To a solution of 1-(4-chlorophenyl)cyclobutanecarbaldehyde (10.0 g, 51.0 mmol), was added (R)-t-butansulfinamide (5.0 g, 41.0 mmol) in THF (60 mL), Ti(OEt)₄ (46.8 g, 205 mmol). The reaction mixture was stirred at room temperature for 3 h and poured into ice-water. The solid was filtered off, and the filtration was extracted with ethyl acetate. The extracts were dried over magnesium sulfate and the solvent was removed on rotovapor. The residue was purified by chromatography on silica gel eluting with heptane/ethyl acetate=9/1 (v/v) to give 11.2 g of product in 92% yield. ¹H NMR (CDCl₃/TMS): δ 8.03 (s, 1H), 7.30 (d, J=8.4 Hz, 2H), 7.10 (d, J=8.4 Hz, 2H), 2.85-2.60 (m, 2H), 2.60-2.40 (m, 2H), 2.15-1.85 (m, 2H), 1.19 (s, 9H). ¹³CNMR(CDCl₃): δ 170.6, 142.5, 132.5, 128.7, 127.5, 57.0, 51.8, 31.1, 30.8, 22.3, 15.9. Anal. Calcd for C₁₅H₂₀ClNOS: C, 60.49; H, 6.77; N, 4.70. Found: C, 60.61; H, 6.80; N, 4.64.

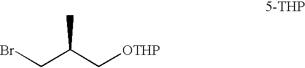

(S)-2-(3-Bromo-2-methylpropoxy) Tetrahydropyran

To the solution of (S)-3-bromo-2-methylpropanol (15.0 g, 98 mmol) in dichloromethane (20 mL) at 0° C., was added 3,4-dihydro-2H-pyran (10.0 g, 119 mmol) and p-toluenesulfonic acid monohydrate (0.19 g). The mixture was stirred at rt overnight and distilled under vacuum to give 6.0 g of product as a colorless oil (26% yield). ¹H NMR (CDCl₃/TMS): δ 4.60 (m, 1H), 3.86 (m, 1H), 3.69 (m, 1H), 3.60-3.40 (m, 3H), 3.33 (m, 1H), 2.11 (m, 1H), 1.95-1.40 (m, 6H), 1.05 (d, J=6.8 Hz, 1.5 Hz), 1.04 (d, J=6.8 Hz, 1.5 Hz). ¹³C NMR (CDCl₃): δ 99.3, 98.5, 70.0, 69.7, 62.3, 61.9, 38.3, 38.1, 35.6, 30.6, 25.4, 19;5, 19.3, 15.9, 15.8.

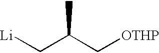

(R)-3-(Tetrahydropyran-2-yloxyl)-2-methylpropyl-lithium

To the suspension of lithium (0.3 g, 43.2 mmol) in ether (5 mL) at 0° C., (S)-2-(3-bromo-2-methylpropoxy) tetrahydropyran (4.22 g, 17.8 mmol) in ether (7 mL) was slowly added. After the reaction was initiated, the reaction mixture was stirred at −10 to −5° C. while the rest of the bromide was added within 1.5 hour. After the addition of the bromide was complete, the reaction mixture was stirred at −10 to −5° C. for 1 hour. The concentration of this lithium reagent was 0.77 M and the yield is 51%.

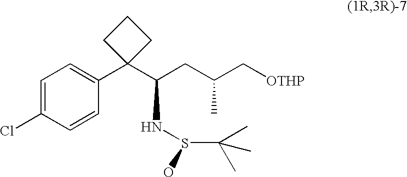

(1R,3R)-N-{1-[1-(4-Chlorophenyl)cyclobutyl]-3-methyl-4-(tetrahydropyran-2-yloxy)butyl}-(R)-t-butanesulfinamide

The reaction of organolithium with tert butyl-sulfinamide was carried out under various conditions. Typical procedure: To the solution of sulfinamide (0.595 g, 2.0 mmol) in THF (10 mL) at −78° C., was added Al(Oct)₃ (5 mL, 25 wt % in hexane, 2.4 mmol). After the mixture was stirred at −78° C. for 5 min., organolithium (5.2 mL, 0.77 M in ether, 4 mmol) was added. The reaction mixture was stirred at −78° C. for 2 hours and then quenched with methanol (5 mL). The reaction mixture was allowed to warm to room temperature, diluted with TBME, washed with brine, and dried over anhydrous MgSO₄. A small amount of the crude product was treated with 2 N HCl in methanol and HPLC analysis of free amino alcohols showed the diastereoselectivity of this reaction is 98.6:1.4. After removal of the solvents, the crude product was purified by chromatography on silica gel (eluting with 5% ethyl acetate in heptane) to give 0.75 g of product in 82% yield. ¹H NMR (CDCl₃/TMS): δ 7.27 (m, 4H), 4.46 (m, 1H), 3.80-3.60 (m, 1H), 3.44 (m, 3H), 3.08 (m, 1H), 2.87(d, J=10.4 Hz, 1H), 2.71 (m, 1H), 2.41 (m, 2H), 2.14 (m, 1H), 2.0-1.3 (m, 10H), 1.16 (s, 9H), 1.0-0.8 (m, 4H). ¹³C NMR (CDCl₃): δ 142.1, 131.9, 130.0, 127.7, 98.7, 98.5, 73.1, 73.0, 62.9, 62.0, 61.8, 56.6, 50.7, 35.5, 35.4, 34.6, 32.2, 30.5, 29.5, 25.4, 22.8, 19.4, 19.2, 16.0, 15.2.

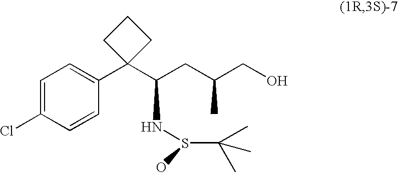

(1R,3S)-N-{1-[1-(4-Chlorophenyl)cyclobutyI]-4-hydroxy-3-methyl-butyl}-(R)-t-butytanesulfinamide

To the solution of (R-3-bromo-2-methylpropanol (15.3 g, 100 mmol) in THF (30 mL) at −35° C., was added i-PrMgCl (51 mL, 2.0 M in THF, 102 mmol) via syringe. After the addition was complete, the reaction mixture was continued to stir at 0° C. for 1 hour. The resulting magnesium salt solution was then slowly added into a suspension of magnesium turnings (4.0 g, 165 mmol) in THF (20 mL). After the reaction was initiated, the reaction mixture was maintained with an inner temperature of 40-50° C. while the solution of magnesium salt in THF was added. After the addition was complete, the reaction mixture was stirred at ambient temperature for 2 h.

To the solution of sulfinamide (5.95 g, 20.0 mmol) in THF (60 mL) at 0° C., was added the Grignard reagent (58 mL, 0.7 M in THF, 40.6 mmol). The reaction mixture was allowed to warm to room temperature and completed in 2 hours. The reaction mixture was quenched by the addition of water (20 mL), and extracted with TBME. The extracts were dried over MgSO₄. After the solvent was removed, a small amount of the crude product was treated with 2 N HCl in methanol. HPLC analysis of the free amino alcohols showed the diastereoselectivity of this reaction is 40.5:59.5. The major diastereomer (4.24 g) and the minor diastereomer (2.82 g) were isolated in 95% combined yield by chromatography on silica gel, eluting with a mixture of ethyl acetate/heptane=6/4(v/v). Major diastereomer: ¹H NMR (CDCl₃/TMS): δ 7.30-7.20 (m, 4H), 3.65-3.45 (m, 3H), 2.94 (d, J=10.2 Hz, 1H), 2.75-2.60 (m, 1H), 2.45-2.30 (m, 2H), 2.30-1.50 (m, 6H), 1.18 (s, 9H), 0.88 (d, J=6.8 Hz, 3H), 0.58 (m, 1H). ¹³C NMR (CDCl₃): δ 142.1, 132.0, 130.1, 127.9, 66.2, 63.5, 56.7, 50.7, 35.6, 34.5, 32.2, 31.6, 22.9, 18.5, 15.2. Anal. Calcd for C₁₉H₃₀ClNO₂S: C, 61.35; H, 8.13; N, 3.77. Found: C, 61.32; H, 8.26; N, 3.40.

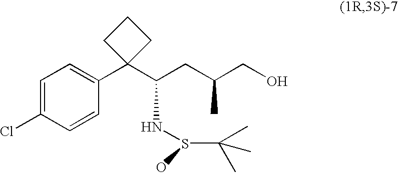

(1S,3S)-N-{1-[1-(4-Chlorophenyl )cyclobutyl-4-hydroxy-3-methyl-butyl}-(R)-t-butanesulfinamide

To a solution of (R)-3-bromo-2-methylpropanol (6.0 g, 39 mmol) in ether (20 mL) at −25° C., was added i-PrMgCl (20 mL, 2.0 M in ether, 40.0 mmol) via syringe. After the addition was complete, the reaction mixture was continued to stir at 0° C. for 1 h. The resulting magnesium salt solution was then slowly added into a suspension of magnesium turnings (1.46 g, 60.0 mol) in ether (5 mL). After the reaction was initiated, the ether solution was gently refluxed. After the addition was complete, the reaction mixture was stirred at ambient temperature for 2 h. The solution of the Grignard reagents became two phases. After vigorously stirring, an aliqutor was taken and titrated to measure the concentration. The Grignard reagent was used for the addition reaction under various conditions and the results are shown in Table 1 (entries 1-3). The following procedure (Table 1, entry 1) is resentative: To the solution of sulfinamide (2.98 g, 10.0 mmol) in dichloromethane (100 mL) at 0° C. was added the Grignard reagent (53 mL, 0.38 M in ether, 20.0 mmol). The reaction mixture was allowed to warm to room temperature and stirred for 24 hours. The reaction mixture was quenched by addition of water (10 mL) and extracted with TBME. The extracts were dried over MgSO₄. After the solvent was removed, a small amount of the crude product was treated with 2 N LiCl in methanol. HPLC analysis of free amino alcohols showed the diastereoselectivity of this reaction is 96.4:3.6. The major diastereomer (2.75 g) and the minor diastereomer (0.10 g) were isolated in 77% yield by chromatography on silica gel, eluting with ethyl acetate/heptane (6:4). Major Diastereomer: ¹H NMR (CDCl₃/TMS): δ 7.27 (d, J=8.4 Hz, 2H), 7.07 (d, J=8.4 Hz, 2H), 3.60-3.3 8 (m, 2H), 3.15-2.90 (m, 3H), 2.45-1.70 (m, 7H), 1.40-0.95 (m, 2H), 1.25 (s, 9H), 0.92 (d, J=6.8 Hz, 3H). ¹³C NMR (CDCl₃): δ 143.9, 132.3, 129.1, 128.1, 68.7, 64.8, 57.2, 51.2, 37.1, 33.3, 32.5, 32.2, 23.4, 17.7, 15.2. Anal. Calcd for C₁₉H₃₀ClNO₂S: C, 61.35; H, 8.13; N, 3.77. Found: C, 61.25; 11, 8.42; N, 3.28.

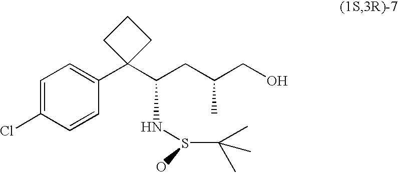

(1S,3R)-N-{1-[1-(4-Chlorophenyl)cyclobutyl]-4-hydroxy-3-methyl-butyl}-(R)-t-butanesulfinamide

To a solution of (S)-3-bromo-2-methylpropanol (15.3 g, 0.10 mol) in ether (50 mL) at −25° C., was added i-PrMgCl (51 mL, 2.0 M in ether, 0.102 mol) via syringe. After the addition was complete, the reaction mixture was continued to stir at 0° C. for 1 h. The resulting magnesium salt solution was then slowly added into a suspension of magnesium turnings (4.0 g, 0.16 mol) in ether (30 mL). After the reaction was initiated, the ether solution was gently refluxed. After the addition was complete, the reaction mixture was stirred at ambient temperature for 2 h. The solution of the Grignard reagents became two phases. After vigorously stirring, an aliqutor was taken and titrated to measure the concentration. The Grignard reagent was used for the addition reaction under various conditions and the results are shown in Table 1 (entries 6-10). The following procedure (Table 1, entry 8) is representative: To the solution of sulfinamide (4.0 g, 13.4 mmol) in dichoromethane (110 mL) at 0° C., Grignard reagent (54 mL, 0.5 M in ether, 27.0 mmol) was added. The reaction was allowed to warm to at room temperature and completed in 10 hours. The reaction mixture was quenched by addition of water (10 mL), and extracted with TBME. The extracts were dried over MgSO₄. After the solvent was removed, a small amount of the crude product was treated with 2 N HCl in methanol. HPLC analysis of free amino alcohols showed the diastereoselectivity of this reaction is 90.6:9.4. The major diastereomer (3.82 g) and the minor diastereomer (0.40 g) were isolated in 87% combined yield by chromatography on silica gel, eluting with ethyl acetate/heptane (6/4). Major Diastereomer: ¹H NMR (CDCl₃/TMS): δ 7.27 (d, J=8.2 Hz, 2H), 7.11 (d, J=8.2 Hz, 2H), 4.04 (m, 1H), 3.70-3.40 (m, 2H), 3.23 (brs, 1H), 2.72 (d, J=8.2 Hz, 1H), 2.35 (m, 2H), 2.16 (m, 2H), 2.04 (m, 2H), 1.92-1.70 (m, 1H), 1.54 (m, 1H), 1.28 (s, 9H), 0.95-0.65 (m, 4H). ¹³C NMR (CDCl₃): δ 143.6, 131.8, 129.0, 127.6, 68.4, 61.5, 56.5, 51.0, 37.8, 32.2, 31.8, 30.7, 23.1, 18.8, 15.0. Anal. Calcd for C₁₉H₃₀ClNO₂S: C, 61.35; H, 8.13; N, 3.77. Found: C, 61.41; H, 8.17; N, 3.60. The absolute configuration of 15 was assigned by x-ray of the crystals.

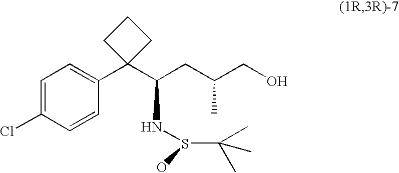

(1R,3R)-N-{1-[1-(4-Chlorophenyl)cyclobutyl]-4-hydroxy-3-methyl-butyl}-(R)-t-butanesulfinamide

To the solution of (S)-3-bromo-2-methylpropanol (6.0 g, 39 mmol) in THF (10 mL) at -35° C., was slowly added i-PrMgCl (20 mL, 2.0 M in THF, 40 mmol) via syringe. After the addition was complete, the reaction mixture was continued to stir at 0° C. for 1 h. The resulting magnesium salt solution was then slowly added into a suspension of magnesium turnings (1.46 g, 60 mmol) in THF (10 mL). After the reaction was initiated, the reaction mixture was stirred at an inner temperature of 40-50° C. while the solution of magenesium salt in THF was added. After the addition was complete, the reaction mixture was stirred at ambient temperature for 2 h. This homogeneous solution was then used for the addition reaction under various conditions and the results are shown in Table 1 (entries 12, 13). The following procedure (Table 1, entry 12) is a representative: To the solution of sulfinamide (4.0 g, 13.4 mmol) in THF (90 mL) at 0° C., Grignard reagent (35 mL, 0.6 M in THF, 21.0 mmol) was added. The reaction was allowed to warm to room temperature and completed in 2 hours. The reaction mixture was quenched by addition of water (10 mL), and extracted with TBME. The extracts were dried over MgSO₄. After the solvent was removed, a small amount of the crude product was treated with 2 N HCl in methanol. HPLC analysis of free amino alcohols showed the diastereoselectivity of this reaction is 15.3:85.7. The major diastereomer (3.74 g) and the minor diastereomer (0.67 g) were isolated in 88% combined yield by chromatography on silica gel, eluting with ethyl acetate/heptane (614). Major Diastereomer: ¹H NMR (CDCl₃/TMS): δ 7.29 (m, 4H), 3.55-3.30 (m, 3H), 2.94 (d, J=10.2 Hz, 1H), 2.75-2.65 (m, 1H), 2.47-2.35 (m, 2H), 2.20-2.10 (m, 1H), 2.00-1.80 (m, 4H), 1.17 (s, 9H), 0.94 (d, J=6.7 Hz, 3H), 1.25-0.85 (m, 2H). ¹³C NMR (CDCl₃): δ 142.1, 132.0,130.1, 127.9, 68.5, 63.0, 56.7, 50.7, 34.9, 34.6, 32.3, 31.9, 22.8, 15.6, 15.2. Anal. Calcd for C₁₉H₃₀ClNO₂S: C, 61.35; H, 8.13; N, 3.77. Found: C, 61.17; H, 8.36; N, 3.44.

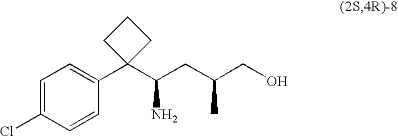

(2S,4R)-4-Amino-4-[1-(4-chlorophenyl)cyclobutyl]-2-methyl-butan-1-ol

To the solution of hydroxy sulfinamide (3.50 g, 9.4 mmol) in methanol (50 mL) at rt, was added the solution of HCl in i-PrOH (10 mL, 5-6 N). The reaction mixture was stirred at room temperature overnight. After the solvent was removed under vacuum, the residue was purified by chromatography on silica gel, eluting with heptane/ethylacetate/triethylamine (1/9/0.2) to give the amino alochol 2.52 g in 100% yield. ¹H NMR (CDCl₃/TMS): δ 7.31 (d, J=8.4 Hz, 2H), 7.07 (d, J=8.4 Hz, 2H), 3.49 (m, 2H), 3.14 (d, J=10.9 Hz, 1H), 2.64 (brs, 3H), 2.50-2.10 (m, 4H), 2.10-1.80(m, 3H), 1.64-1.57 (m, 1H), 1.01 (d, J=7.1 Hz, 3H), 0.78 (m, 1H). ¹³C NMR(CDCls): δ 143.6, 131.8, 128.6, 127.8, 66.7, 53.4, 51.2, 36.6, 32.9, 31.7, 31.4, 16.1,15.0.

A mixture of amino alcohol (1.0 g, 3.74 mmol) and D-tartaric acid (0.56 g, 3.74 mmol) was dissolved into methanol (20 mL). To the resulting solution, TBME (50 mL) was added and solid was precipitated out. The salt was filtered and dried in a vacuum oven (<40° C.). 1.37 g of the D-tartaric acid salt was obtained (88% yield). ¹H NMR (DMSO): δ 7.43 (d, J=7.7 Hz, 2H), 7.31 (d, J=7.7 Hz, 2H), 6.89 (brs, 6H), 3.98 (d, J=3.8 Hz, 2H), 3.65 (d, J=10.6 Hz, 1H), 3.33 (s, 2H), 2.55-2.40 (m, 1H), 2.40-2.20 (m, 3H), 2.05-1.85 (m, 1H), 1.80-1.65 (m, 2H), 1.54 (m, 1H), 0.80 (d, J=6.3 Hz, 3H), 0.90-0.70 (m, 1H). ¹³C NMR (DMSO): δ 175.1, 142.8, 131.9, 130.4, 128.6, 72.4, 65.6, 56.2, 49.3, 33.9, 32.5, 32.0, 31.5, 18.9, 15.6. Anal. Calcd for C₉H₂₈ClNO₇: C, 54.61; H, 6.75; N, 3.35. Found: C, 54.17; H, 7.41; N, 2.87.

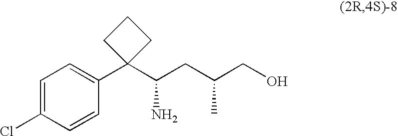

(2R, 4S)-4-Amino-4-[1-(4-chlorophenyl)cyclobutyl]-2-methyl-butan-1-ol

To the solution of hydroxy sulfinamide (3.36 g, 9.0 mmol) in methanol (50 mL) at room temperature, the solution of HCl in i-PrOH (10 mL, 5-6 N) was added. The reaction mixture was stirred at room temperature overnight. After the solvent was removed under vacuum, the residue was purified by chromatography on silica gel, eluting with heptane/ethyl acetate/triethylamine (1/9/0.2) to give the amino alcohol: 2.35 g in 97% yield. The ¹H and ¹³C NMR are identical to amino alcohol above.

A mixture of amino alcohol (1.0 g, 3.74 mmol) and D-tartaric acid (0.56 g, 3.74 mmol) was dissolved into methanol (20 mL) by heating. After it was cooled down, solid was precipitated out. The salt was filtered and dried in a vacuum oven (<40° C.). 1.28 g (82% yield) of the D-tartaric acid salt was obtained. ¹H NMR (DMSO): δ 7.43 (d, J=8.5 Hz, 2H), 7.28 (d, J=8.5 Hz, 2H), 6.92 (brs, 6H), 3.98 (s, 2H), 3.57 (d, J=9.0 Hz, 1H), 3.33 (d, J=5.0 Hz, 2H), 2.55-2.20 (m, 5H), 2.05-1.85 (m, 1H), 1.80-1.50 (m, 3H), 0.81 (d, J=6.5 Hz, 3H), 0.80-0.60 (m, 1H). ¹³C NMR (DMSO): δ 175.0, 142.9, 131.9, 130.3, 128.6,72.0, 65.5, 56.3, 49.4, 34.0, 32.4, 32.2, 31.5, 18.9, 15.6. Anal. Calcd for C₁₉H₂₁ClNO₇: C, 54.61; H, 6.75; N, 3.35. Found: C, 54.54; H, 6.78; N, 3.20.

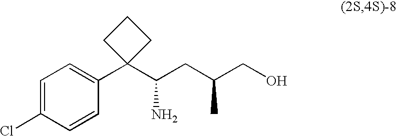

(2S, 4S)-4-Amino-4-[1-(4-chlorophenyl)cyclobutyl]-2-methyl-butan-1-ol

To the solution of hydroxy sulfinamide (3.21 g, 8.6 mmol) in methanol (50 mL) at room temperature, the solution of HCl in i-PrOH (10 mL, 5-6 N) was added. The reaction mixture was stirred at room temperature overnight. After the solvent was removed under vacuum, the residue was purified by chromatography on silica gel, eluting with heptane/ethyl acetate/triethylamine (1/9/0.2) to give amino alcohol:2.30 g in 100% yield. ¹H NMR (CDCl₃/TMS): δ 7.31 (d, J=8.3 Hz, 2H), 7.06 (d, J=8.4 Hz, 2H), 3.49 (dd, J=3.2, 11.5 Hz, 1H), 3.30-2.80 (m, 5H), 2.50-2.10 (m, 4H), 2.10-1.60 (m, 3H), 1.64-1.57 (m, 1H), 0.85 (d, J=6.8 Hz, 3H), 0.46-0.35 (m, 1H). ¹³C NMR (CDCl₃): δ 143.7, 132.2, 128.9, 128.1, 69.2, 58.7, 51.7, 40.1, 37.9, 32.0, 31.8, 19.5, 15.1.

The mixture of amino alcohol (1.0 g. 3.74 mmol) and D-tartaric acid (0.56 g, 3.74 mmol) was dissolved into methanol (20 mL) by heating. After it was cooled down, solid was precipitated out. The salt was filtered and dried in a vacuum oven (<40° C.). 1.25 g (77% yield) of the tartrate salt was obtained. ¹H NMR (DMSO): δ 7.43 (d, J=7.2 Hz, 2H), 7.31 (d, J=7.2 Hz, 2H), 6.74 (brs, 6H), 3.98 (s, 2H), 3.49 (d, J=10.1 Hz, 1H), 3.25-3.08 (m, 2H), 2.60-2.40 (m, 1H), 2.40-2.15 (m, 3H), 2.05-1.85 (m, 1H), 1.80-1.58 (m, 2H), 1.22 (m, 1H), 1.06 (m, 1H), 0.84 (d, J=5.9 Hz, 3H). ¹³C NMR (DMSO): δ 175.1, 143.0, 131.9, 130.4, 128.6, 72.4, 67.3, 56.3, 49.4, 33.7, 32.3, 32.0, 16.8, 15.5. Anal. Calcd for C₁₉H₂₈ClNO₇—H₂O: C, 52.35; H, 6.94; N, 3.21. Found: C, 52.73; 11, 6.76; N, 3.11.

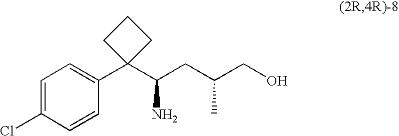

(2R, 4R)-4-Amino-4-[1-(4-chlorophenyl)cyclobutyl]-2-methyl-butan-1-ol

To the solution of amino alcohol (3.08 g, 8.3 mmol) in methanol (50 mL) at room temperature, the solution of HCl in i-PrOH (10 mL, 5-6 N) was added. The reaction mixture was stirred at room temperature overnight. After the solvent was removed under vacuum, the residue was purified by chromatography on silica gel, eluting with heptane/ethyl acetate/triethylamine (1/9/0.2) to give amino alcohol: 2.22 g in 99% yield. The ¹H and ¹³C NMR are identical to amino alcohol (2S,4S)-6.

The mixture of amino alcohol (1.0 g, 3.74 mmol) and D-tartaric acid (0.56 g, 3.74 mmol) was dissolved into methanol (20 mL) by heating. After it was cooled down, solid was precipitated out. The salt was filtered and dried in a vacuum oven (<40° C.). 1.27 g (81% yield) of the tartrate salt was obtained. ¹H NMR (DMSO): δ 7.43 (d, J=8.6 Hz, 2H), 7.28 (d, J=7.2 Hz, 2H), 7.03 (brs, 6H), 3.92 (s, 2H), 3.45 (d, J=10.5 Hz, 1H), 3.25-3.07 (m, 2H), 2.48 (m, 1H), 2.29 (m, 3H), 1.95 (m, 1H), 1.80-1.55 (m, 2H), 1.21 (m, 1H), 1.03 (m, 1H), 0.84 (d, J=6.5 Hz, 3H). ¹³C NMR (DMSO): δ 175.0, 143.1, 131.9, 130.3,128.6, 72.2, 67.3, 56.4, 49.4, 33.8, 32.4, 16.8, 15.5. Anal. Calcd for C₁₉H₂₈ClNO₇: C, 54.61; H, 6.75; N, 3.35. Found: C, 54.68; H, 6.87; N, 3.20.

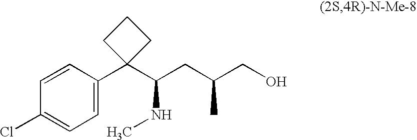

(2S, 4R)-4-Methylamino-4-[1-(4-chlorophenyl)cyclobutyl]-2-methyl-butan-1-ol

A solution of amino alcohol (2.52 g, 9.4 mmol) with formic acid (4.3 g, 94 mmol) in toluene (30 mL) was heated to reflux for 5 h. After the solvent was removed on rotary evaporator, THF (20 mL) and borane-THF (20 mL, 1.0 M in THF) were added under argon at 0° C. After the reaction mixture was stirred at room temperature for 24 h, the reaction was quenched with 2 N NaOH. After separation, the aqueous phase was extracted with TBME. The organic phase was dried over NaSO₄. After the solvent was removed, the residue was isolated by chromatography on silica gel, eluting with heptane/ethyl acetate/triethyl amine (1/9/0.2) to give 5:1.79 g (68% yield). ¹H NMR (CDCl₃/TMS): δ 7.34 (d, J=8.6 Hz, 2H), 7.24 (d, J=8.6 Hz, 2H), 3.45-3.30 (m, 2H), 3.21 (brs, 2H), 2.78 (d, J=9.9 Hz, 1H), 2.55 (s, 3H), 2.60-2.15 (m, 4H), 1.95-1.70 (m, 3H), 1.50-1.40 (m, 1H), 0.93 (d, J=7.1 Hz, 3H), 0.95-0.80 (in, 1H). ¹³C NMR (CDCl₃): δ 142.5, 132.0, 129.0, 128.0, 66.8, 63.1, 52.2, 35.6, 35.5, 35.1, 33.4, 32.9, 17.0, 16.2.

To the flask containing the amino alcohol (1.0 g, 3.55 mmol), was added LiCl (15 mL, 2.0 M in ether) and anhydrous ethanol (1 mL). After the mixture was stirred at room temperature for 30 min, the white solid was formed and filtered. The solid was dried under vacuum. 0.8 g (62% yield) of the HCl ethanol solvate (C₁₆H₂₄ClNO-EtOH-HCl) was obtained. ¹H NMR (DMSO): δ 8.62 (brs, 1H), 8.23 (brs, 1H), 7.47 (m, 4H), 4.68 (brs, 1H), 4.40 (brs, 1H), 3.60 (m, 1H), 3.46 (q, J=6.7 Hz, 2H), 3.26 (m, 2H), 2.56 (s, 3H), 2.70-2.20 (m, 4H), 1.94 (m, 1H), 1.80-1.40 (m, 3H), 1.07 (t, J=6.7 Hz, 3H), 1.00 (m, 1H), 0.88 (d, J=5.3 Hz, 3H). ¹³C NMR (DMSO): δ 142.2, 132.2,130.8, 128.8, 66.3, 64.8, 56.7, 49.9, 33.7, 33.5, 33.1, 32.5, 19.3, 18.0, 16.2.

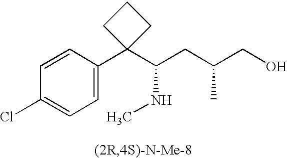

(2R, 4S)-4-Methylamino-4-[1-(4-chlorophenyl)cyclobutyl]-2-methyl-butan-1-ol

A solution of amino alcohol (2.0 g, 7.5 mmol) and formic acid (3.45 g, 75 mmol) in toluene (30 mL) was heated to reflux for 5 h. After the solvent was removed on rotary evaporator, the residue was dissolved into THF (20 mL). Borane.THF (20 mL, 1.0 M in THF) was added under argon at 0° C. After being stirred at room temperature for 24 h, the reaction mixture was quenched with 2 N NaOH. After separation, the aqueous phase was extracted with TBME. The organic phase was dried over NaSO₄. After the solvent was removed, the residue was isolated by chromatography on silica gel, eluting with heptane/ethyl acetate/triethyl amine(1/9/0.2) to give 6:1.41 g (67% yield).

To the flask containing the amino alcohol (1.0 g, 3.55 mmol), was added HCl (15 mL, 2.0 M in ether) and anhydrous ethanol (1 mL). After the mixture was stirred at room temperature for 30 min, the white solid formed was filtered and dried under vacuum. 0.8 g (62% yield) of HCl salt (C₁₆H₂₄ClNO-EtOH-HCl) was obtained. The ¹H NMR and ¹³C NMR of amino alcohol as well as the HCl salt are identical to those of amino alcohol above.

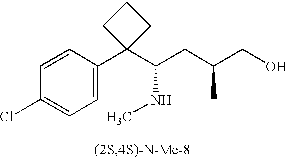

(2S, 4S)-4-Methylamino-4-[1-(4-chlorophenyl)cyclobutyl]-2-methyl-butan-1-ol

A solution of amino alcohol 3 (2.30 g, 8.6 mmol) and formic acid (4.0 g, 86 mmol) in toluene (30 mL) was heated to reflux for 5 h. After the solvent was removed on rotary evaporator, the residue was dissolved into THF (20 mL). Borane-THF (20 mL, 1.0 M in THF) was added under argon at 0° C. After the reaction mixture was stirred at room temperature for 24 h, the reaction was quenched with 2 N NaOH. After separation, the aqueous phase was extracted with TBME. The organic phase was dried over NaSO₄. After the solvent was removed, the residue was isolated by chromatography on silica gel, eluting with heptane/ethyl acetate/triethyl aniine=1/9/0.2 to give 1.79 g of ?? (74% yield). ¹H NMR (CDCl₃/TMS): δ 7.35 (d, J=8.6 Hz, 2H), 7.25 (d, J=8.6 Hz, 2H), 3.60 (brs, 2H), 3.45 (m, 1H), 3.07 (m, 1H), 2.73 (d, J=10.3 Hz, 1H), 2.56 (s, 3H), 2.60-2.15 (m, 4H), 1.95-1.70 (m, 3H), 1.50 (m, 1H), 0.86 (d, J=7.0 Hz, 3H), 0.60-0.47 (m, 1H). ¹H NMR(CDCl₃): δ 141.9, 132.1, 129.0, 128.1, 68.9, 68.4, 52.4, 39.1, 37.3, 36.2, 35.3, 33.6, 19.2, 17.1.

To the flask containing 7 (1.0 g, 3.55 mmol), was added the solution of D-tartaric acid (0.53 g, 3.55 mmol) in methanol (15 mL). The solution was concentrated to about 5 mL by evaporation and put in refrigerator for crystallization. The solid was filtered and dried under vacuum. 1.38 g (85% yield) of the D-tartrate salt (C₁₆H₂₄ClNO-C₄H₆O₆-1.5H₂O) was obtained. ¹H NMR (DMSO): δ 7.42 (d, J=8.7 Hz, 2H), 7.37 (d, J=8.7 Hz, 2H), 5.85 (brs, 6H), 4.00 (s, 2H), 3.30-3.20 (m, 2H), 3.11 (m, 1H), 2.52 (s, 3H), 2.60-2.20 (m, 4H), 1.91 (m, 1H), 1.80-1.50 (m, 2H), 1.18 (m, 2H), 0.82 (d, J=6.6 Hz, 3H). ¹³C NMR (DMSO): δ 174.9, 143.2, 131.9, 130.5, 128.6, 72.4, 66.9, 64.5, 50.2, 34.3, 34.1, 33.0, 32.8, 32.7, 17.1, 16.0. Anal. Calcd for C₂₀H₃₀ClNO₇-1.5H₂O: C, 52.34; H, 7.25; N, 3.05. Found: C, 52.25; H, 7.30; N, 2.90.

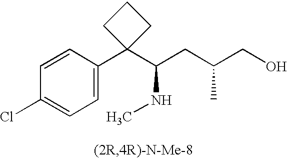

(2R, 4R)-4-Methylamino-4-[1-(4-chlorophenyl)cyclobutyl-2-methylbutan-1-ol

A solution of amino alcohol (1.84 g, 6.89 mmol) with formic acid (3.2 g, 70 mmol) in toluene (30 mL) was heated to reflux for 5 h. After the solvent was removed by rotary evaporator, the residue was dissolved into THF (20 mL) and Borane-THF (20 mL, 1.0 M in THF) was added under argon at 0° C. After the reaction mixture was stirred at room temperature for 24 h, the reaction was quenched with 2 N NaOH. After separation, the aqueous phase was extracted with TBME. The organic phase was dried over NaSO₄. After the solvent was removed, the residue was isolated by chromatography on silica gel, eluting with heptane/ethyl acetate/triethyl amine 1/9/0.2(v/v/v) to give 0.88 g of 8 (45% yield).

To a flask containing 8 (0.81 g, 2.9 mmol), was added the solution of L-tartaric acid (0.44 g, 2.9 mmol) in methanol (15 mL). The mixture was concentrated to about 5 mL by evaporation and standed in a refrigerator for crystallization. The solid was filtered and dried under vacuum. 1.10 g (83% yield) of the L-tartrate salt (C₁₆H₂₄ClNO—C₄H₆O₆-1.5H₂O) was obtained. The ¹H NMR and ¹³C NMR of amino alcohol: 8 as well as the tartaric acid salt are identical to those of amino alcohol 7. Anal. Calcd for C₂₀H₃₀ClNO₇-1.5H₂O: C, 52.34; H, 7.25; N, 3.05. Found: C, 52.47; H, 7.15; N, 2.93.

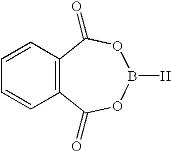

To a dried 100 mL three-neck flask, equipped with a thermometer and an outlet, was added phthalic acid (4.58 g, 27.6 mmol) and THF (32 mL, degassed with Ar) under argon. The mixture was stirred at room temperature for 10 mm to dissolve the solid. After cooling to −20° C., BH₃.THF (27.6 mL, 1 M in THF, 27.6 mmol) added via syringe. The mixture was stirred at −20° C. for approximately 30 min until the evolution of hydrogen ceased. The resulting homogeneous solution was cooled to −78° C. and used for next reaction.

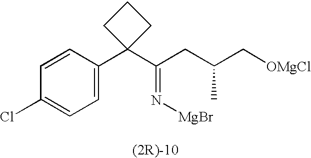

(2R)-4-Imino-2-methyl-4-[1-(4-chlorophenyl)-cyclobutyl]-butan-1-ol Magnesium Salt

A 100 mL three-neck flask, equipped with a thermomate, a reflux condenser and an addition funnel, was dried and flashed with argon. Isopropylmagnesium chloride (2.0 M in Et₂O, 3.3 mL, 6.6 mmol) was charged and cooled to −25° C. (S)-3-Bromo-2-methyl-propan-1-ol (1.0 g, 6.54 mmol) in Et₂O (5 mL) was added over 15 mm. The mixture was allowed to warm to ambient temperature over 1 h to form a homogeneous solution. Magnesium turnings (300 mg, 12.5 mmol) were added in one portion, and the mixture was stirred without heating for 1 h. The reaction was exothermic and the mixture refluxed. The internal temperature fell gradually to 25° C. when the reaction ceased. The solution was titrated (0.6 M, 80% yield). A solution of 1-(4-chloro-phenyl)-cyclobutanecarbonitrile (880 mg, 4.6 mmol) in THF (2 mL) was added dropwise. The reaction was stirred at ambient temperature for 2 h, and cooled to −78° C. for next reaction.

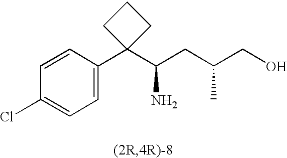

(2R,4R)-4-Methylamino-4-[1-(4-chloro-phenyl)-cyclobutyl]-2-methyl-butan-1-ol

The precooled solution of magnesium salt (R)-10 was added dropwise to the above phthalic borane solution at −78° C. The addition rate was controlled so that the internal temperature was kept below −70° C. The mixture was stirred at -78° C. for 1 h. The reaction was monitored by HPLC. After the reaction was complete, 3 N NaOH (5 mL) was added to quench the reaction. The mixture was warmed to ambient temperature and filtered off the white solid. The filtration was extracted with CH₂Cl₂ (3×15 mL) and dried (CaCO₃). Removal of the solvents and purification of the residue by flash chromatography on silica gel (elute with AcOEt-MeOH-Et₃N 90:9:1) gave a mixture of diasteromers in the ratio of 98:2, favoring (2R,4R)-1-OH DDMS as the major product.

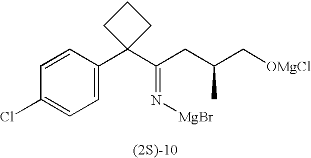

(2S)-4-Imino-2-methyl-4-[1-(4-chlorophenyl)-cyclobutyl]-butan-1-ol Magnesium Salt

The same procedure as described above for the preparation of was applied, except using (R)-3-bromo-2-methyl-propan-1-ol, instead of (S)-3-bromo-2-methyl-propan-1-ol.

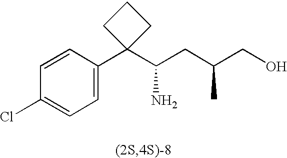

(2S, 4S)-Methylamino-4-[1-(4-chlorophenyl)-cyclobutyl]-2-methyl-butan-1-ol

The same procedure as described above for the preparation of (2R,4R)-1-OH-DDMS was applied, except using (S)-10. The reaction gave a mixture of diasteromers in the ratio of greater than 95:5, favoring (2S,4S)-1-OH DDMS as the major product.

5.1.2. 7-Hydroxyl Experimental Data

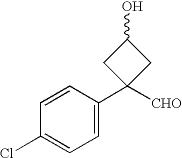

1-(4-Chloro-phenyl)-3-hydroxy-cyclobutanecarbaldehyde

To a solution of 1-(4-chloro-phenyl)-3-hydroxy-cyclobutanecarbonitrile (cis/trans=2.6:1) (25.0 g, 0.12 mol) in THF (100 mL) at 0° C., was slowly added the solution of Dibal (260 mL, 1.0 M in hexane, 0.26 mol). After the reaction mixture was stirred at 0° C. for 1 h, 1H NMR of the reaction mixture showed the reaction was complete. The reaction was then quenched by the addition of 10% aqueous citric acid (200 mL) at −78° C. After the reaction mixture was warmed to room temperature, the solid was filtered off and rinsed with TBME. The organic phase was separated and aqueous phase was extracted with TBME (2×60 mL). The organic layers were combined and dried over magnesium sulfate. The solvent was removed and the residue was purified by silica gel chromatography, eluting with 25% ethyl acetate in heptane to give the a mixture of cis and trans aldehyde (cis/trans 2.3:1): 21.0 g in 83% yield. ¹H NMR (CDCl₃/TMS): d 9.52 (s, 1H), 7.40-7.05 (m, 4H), 4.32 (m, 1H), 3.15-3.08 (m, 0.6H), 2.80-2.70 (m, 1.4H), 2.70-2.60 (m, 1.4H), 2.35-2.25 (m, 0.6H). 13C NMR (CDCl3): d 200.0, 199.7,139.3, 137.7, 133.7, 133.6, 129.4, 129.0, 128.9, 128.2, 63.2, 62.0, 50.5, 49.9, 40.0, 39.9.

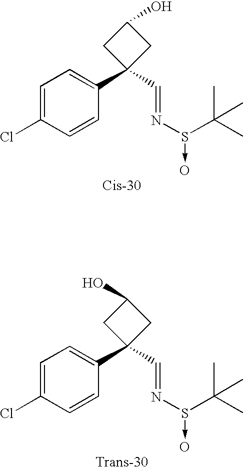

N-(1-(4-Chlorophenyl)-3-hydroxy-cyclobutanemethylene)-(R)-tert-butanesulfinamide

To a solution of 1-(4-chlorophenyl)-3-hydroxy-cyclobutanecarbaldehyde (12.8 g, 61.0 mmol) in THF (20 mL), was added (R)-t-butanesulfinamide (7.4 g, 61.0 mmol), toluene (100 mL) and Ti(OEt)₄ (69.4 g, 304 mmol). The reaction mixture was heated and stirred at 100° C. for 1 h. TLC showed the reaction was complete. The reaction mixture was then poured into ice water and the solid was filtered off. The product was extracted with ethyl acetate and the solution was dried over magnesium sulfate. After the solvent was removed, the residue was purified by silica gel chromatography eluting with heptane/ethyl acetate=1/1(v/v) to give trans-11 (higher Rf): 5.3 g and cis-isomer (lower Rf): 12.1 g in 91% combined yield. Trans-isomer: ¹H NMR (CDCl₃/TMS): δ 7.99 (s, 1H), 7.31 (d, J=8.7 Hz, 2H), 7.08 (d, J=8.7 Hz, 2H), 4.39 (m, 1H), 3.25-3.07 (m, 2H), 2.44-2.35 (m, 2H), 1.72 (brs, 1H), 1.18 (s, 9H). ¹³C NMR (CDCl₃): δ 170.9, 142.4, 133.1, 129.2, 127.8, 62.5, 57.5, 44.2, 42.4, 42.2, 22.6. Cis-11: ¹H NMR (CDCl₃/TMS): δ 8.00 (s, 1H), 7.34 (d, J=8.5 Hz, 2H), 7.21 (d, J=8.7 Hz, 2H), 4.32 (m, 1H), 2.94-2.87 (m, 2H), 2.75-2.60 (m, 2H), 2.35 (brs, 1H), 1.18 (s, 9H). ¹³C NMR (CDCl₃): δ 171.8, 140.5, 133.2, 129.2, 128.7, 63.1, 57.5, 45.1, 42.4, 22.6. Anal. Calcd for C15H20ClNO₂S: C, 57.40; H, 6.42; N, 4.46. Found: C, 57.49; H, 6.44; N, 4.33.

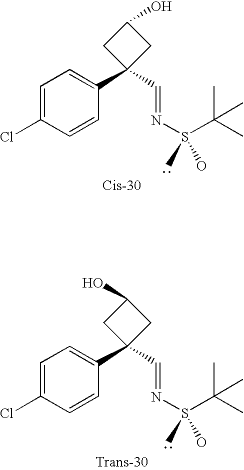

N-(1-(4-Chlorophenyl)-3-hydroxy-cyclobutanemethylene)-(S)-tert-butanesulfinamide

To a solution of 1-(4-chlorophenyl)-3-hydroxy-cyclobutanecarbaldehyde (12.4 g, 59.0 mmol) in THF (20 mL), was added (R)-t-butanesulfinamide (5.95 g, 49.0 mmol), toluene (120 mL) and Ti(OEt)₄ (68.5 g, 0.30 mol). The reaction mixture was heated and stirred at 100° C. for 1 h. TLC showed the reaction was complete. The reaction mixture was then poured into ice water and the solid was filtered off. The product was extracted with ethyl acetate and the solution was dried over magnesium sulfate. After the solvent was removed, the residue was purified by silica gel chromatography eluting with heptane/ethyl acetate=1/1(v/v) to give trans-12 (higher Rf): 4.55 g and cis-12 (lower Rf): 9.67 g in 92% yield. The ¹H NMR and ¹³C NMR data of these compounds are identical to those of the enantiomers: cis-11 and trans-11. Cis-12: Anal. Calcd for C₁₅H₂₀ClNO₂S: C, 57.40; H, 6.42; N, 4.46. Found: C, 57.56; H, 6.47; N, 4.32.

Reaction of cis-30 With Isobutyllithium (Table 2): General Procedure:

To the solution of cis-11 (25 mg, 0.08 mmol) in various solvent (5 mL) at −78° C., was added the solution of isobutyllithium in hexane (3.2 or 2.2 eq.). The reaction mixture was continued to stir at −78° C. The yield and diastereoselectivity of the addition product were obtained by achiral HPLC method.

Procedure for the Reaction in the Presence of Lewis Acid (Entries 4-7):

To the mixture of cis-1 (25 mg, 0.08 mmol) in THF (5 mL) and Lewis acid at −78° C., was added the solution of isobutyllithium (0.18 mL, 1.47 M in hexane, 0.26 mmol). The reaction mixture was continued to stir at −78° C. The yield and diastereoselectivity of the addition product were obtained by achiral HPLC method.

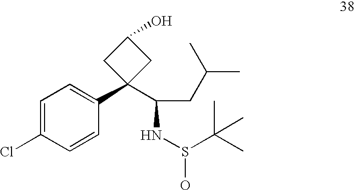

Cis-(1R)-N-{1-[1-(4-chlorophenyl)-3-hydroxycyclobutyl]-3-methylbutyl}-(R)-tert-butanesulfinamide

To the solution of cis-30 (3.28 g, 10.5 mmol) in THF (150 mL) at -78° C., was added boron trifluoride etherate (4.8 g, 4.3 mL, 33.8 mmol). After the mixture was stirred at −78° C. for 10 min, isobutyllithium (23 mL, 1.47 M in hexane, 33.8 mmol) was slowly added. After the addition was complete, the reaction mixture was stirred at −78° C. for 1 h. TLC showed the reaction was complete. The reaction was then quenched by methanol (10 mL) and aqueous saturated sodium bicarbonate (50 mL). The reaction mixture was allowed to warm to room temperature. HPLC showed the ratio of the products was 97.8:2.2. The two phases were separated. The aqueous phase was extracted with TBME (2×30 mL). The organic layers were combined and dried over magnesium sulfate. After solvent was removed, the residue was purified by silica gel chromatography eluting with a mixture of solvents: ethyl acetate/heptane 7/3 (v/v) to give 13: 3.62 g in 93% yield. ¹H NMR (CDCl₃/TMS): δ 7.40-7.25 (m, 4H), 4.01 (m, 1H), 3.41 (m, 1H), 3.00-2.90 (m, 2H), 2.87-2.75 (m, 1H), 2.47-2.39 (m, 1H), 2.11-2.04 (m, 1H), 1.56 (m, 1H), 1.20-0.65 (m, 2H), 1.17 (s, 9H), 0.85 (d, J=6.5 Hz, 3H), 0.82 (d, J=6.7 Hz, 3H). ¹³C NMR (CDCl₃): δ 140.3, 132.5, 131.0, 128.3, 66.3, 61.8, 57.0, 47.0, 43.2, 43.1, 40.6, 24.09, 24.06, 23.0, 21.0. Anal. Calcd for C₁₉H₃₀ClNO₂S: C, 61.35; H, 8.13; N, 3.77. Found: C, 61.41; H, 8.33; N, 3.67.

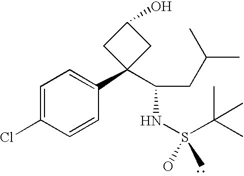

Cis-(1S)-N-{1-[1-(4-chlorophenyl)-3-hydroxycyclobutyl]-3-methylbutyl}-(S)-tert-butanesulfinamide

To the solution of cis-30 (6.86 g, 21.9 mmol) in THF (150 mL) at −78° C., was added boron trifluoride etherate (9.97 g, 8.9 mL, 70.0 mmol). After the mixture was stirred at −78° C. for 10 min, isobutyllithium (60.9 mL, 1.15 M in hexane, 70.0 mmol) was slowly added. After the addition was complete, the reaction mixture was stirred at -78° C. for 1 h. TLC showed the reaction was complete. The reaction was then quenched by methanol (10 mL) and aqueous saturated sodium bicarbonate (50 mL). The reaction mixture was allowed to warm to room temperature. HPLC showed the ratio of the products was 96.4:3.6. The two phases were separated. The aqueous phase was extracted with TBME (2×30 mL). The organic layers were combined and dried over magnesium sulfate. After solvent was removed, the residue was purified by silica gel chromatography eluting with a mixture of solvents: ethyl acetate/heptane=7/3 (v/v) to give 15: 7.88 g in 97% yield. The ¹H NMR and ¹³C NMR data of 15 are identical to those of enantiomer: 13. Anal. Calcd for C₁₉H₃₀ClNO₂S: C, 61.35; H, 8.13; N, 3.77. Found: C, 61.05; H, 8.18; N, 3.63.

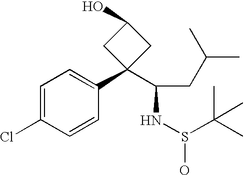

Trans-(1R)-N-{1-[1-(4-chlorophenyl)-3-hydroxycyclobutyl]-3-methylbutyl}-(R)-tert-butanesulfinamide

Reaction of trans-30 with isobutyllithium (Table 3): General Procedure:

To the solution of trans-30 (25 mg, 0.08 mmol) in various solvent (5 mL) at −78° C., was added the solution of isobutyllithium in hexane (3.2 or 2.2 eq.). The reaction mixture was continued to stir at −78° C. The yield and diastereo selectivity of the addition product were obtained by achiral HPLC method. Procedure for the reaction in the presence of Lewis acid (entries 4-6): To the mixture of trans-11 (25 mg, 0.08 mmol) in THF (5 mL) and Lewis acid at −78° C., was added the solution of isobutyllithium (0. 18 mL, 1.47 M in hexane, 0.26 mmol). The reaction mixture was continued to stir at −78° C. The yield and diastereoselectivity of the addition product were obtained by achiral HPLC method. The major product 17 (74% yield, Table 3, entry 1) was isolated by silica gel chromatography, eluting with heptane/ethyl acetate=1/1 (v/v). ¹H NMR (CDCl₃/TMS): δ 7.28 (d, J=8.0 Hz, 2H), 7.09 (d, J=8.0 Hz, 2H), 4.46 (m, 1H), 3.40 (brs, 1H), 3.40-3.25 (m, 2H), 3.05 (d, J=9.8 Hz, 1H), 2.66 (m, 1H), 2.32 (dd, J=7.0, 11.8 Hz, 1H), 2.18 (dd, J=7.0, 12.6 Hz, 1H), 1.59 (m, 1H), 1.36-0.70 (m, 2H), 1.15 (s, 9H), 0.90 (d, J=6.5 Hz, 3H), 0.83 (d, J=6.6 Hz, 3H). ¹³C NMR (CDCl₃): δ 144.0, 132.2, 129.5, 128.3, 62.6, 62.4, 57.1, 44.1, 43.2, 42.6, 42.0, 24.3, 24.1, 23.1, 21.1.

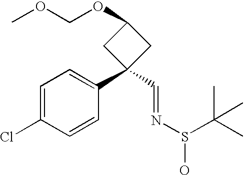

Trans-N-(1-(4-chlorophenyl)-3-methoxymethoxy-cyclobutanemethylene)-(R)-tert-butanesulfinamide

To the mixture of trans-11 (6.45 g, 20.6 mmol) and diisopropylethylamine (28.8 mL, 165.4 mmol) in dichloromethane (100 mL) at 0° C., was added methoxymethyl bromide (10.6 g, 82.0 mmol). The reaction mixture was stirred at room temperature for 24 h. After dichloromethane was removed by rotary evaporator, toluene (30 mL) was added and the mixture was stirred at room temperature for 1 h. TLC checked the reaction mixture and showed about 10% starting material left. To the reaction mixture, was added additional diisopropylethylamine (2.5 mL, 14.3 mmol) and methoxymethyl bromide (1.5 g 12.0 mmol) at 0° C. The reaction mixture was then stirred at room temperature for 12 h. TLC showed no starting material left. After usual work-up, the product: 19 (5.45 g, 74% yield) was isolated by silica gel chromatography eluting with 25% ethyl acetate in heptane. ¹H NMR (CDCl₃/TMS): δ 8.01 (s, 1H), 7.31 (d, J=8.7 Hz, 2H), 7.08 (d, J=8.7 Hz, 2H), 4.61 (s, 2H), 4.22 (m, 1H), 3.36 (s, 3H), 3.20-3.10 (m, 2H), 2.52-2.42 (m, 2H), 1.17 (s, 9H). 13C NMR (CDCl3): d 170.7, 142.4, 133.0, 129.1, 127.7, 95.5, 66.6, 57.5, 55.8, 45.1, 40.0, 22.6. Anal. Calcd for C₁₇H₂₄ClNO₃S: C, 57.05; H, 6.76; N, 3.91. Found: C, 57.11; H, 6.75; N, 3.87.

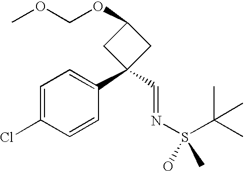

Trans-N-(-(4-chlorophenyl)-3-methoxymethoxy-cyclobutanemethylene)-(S)-tert-butanesulfinamide

To the mixture of trans-12 (4.42 g, 14.1 mmol) and diisopropylethylamine (10.1 mL, 58 mmol) in ether (10 mL) and dichloromethane (10 mL) at 0° C., was added methoxymethyl chloride (2.30 g, 28.6 mmol). The reaction mixture was stirred at room temperature for 16 h. TLC showed no starting material left. After usual work-up, the product: 20 (4.51 g, 89% yield) was isolated by silica gel chromatography eluting with 25% ethyl acetate in heptane. ¹H NMR and ¹³C NMR data 20 is identical to those of enantiomer 19. Anal. Calcd for C₁₇H₂₄ClNO₃S: C, 57.05; H, 6.76; N, 3.91. Found: C, 56.98; H, 6.75; N, 3.80.

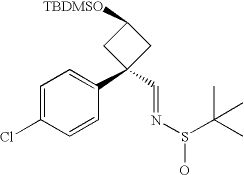

Trans-N-(1-(4-chlorophenyl)-3-tert-butyldimethylsiloxy-cyclobutanemethylene)-(R)-ter t-butanesulfinamide

To the solution of cis-11 (0.116 g, 0.37 mmol) and imidazole (0.101 g, 1.48 mmol) in DMF (10 mL), was added TBDMSCl (0.112 g, 0.74 mmol). After the reaction mixture was stirred at rt for 4 h, the reaction mixture was diluted with ethyl acetate, washed with water, saturated sodium chloride, dried over magnesium sulfate. After solvent was removed, the residue was purified by preparative TLC plate with 10% ethyl acetate as solvent to give 19a: 0. 106 g, 67% yield. ¹H NMR (CDCl₃/TMS): δ 8.02 (s, 1H), 7.31 (d, J=8.7 Hz, 2H), 7.08 (d, J=8.7 Hz, 2H), 4.3 6 (m, 1H), 3.20-3. 10 (m, 1H), 3.10-2.9 5 (m, 1H), 25 2.50-2.35 (m, 211), 1.19 (s, 911), 0.88 (s, 911), 0.06 (s, 611). ¹³C NMR (CDCl₃): δ 171.0, 142.3, 132.7, 128.8, 127.6, 62.2, 57.2, 44.2, 42.6, 42.4, 25.7, 22.4, 17.9,-4.9.

General Procedure:

To the solution of 30 (0.06-0.07 mmol) in THF (2 mL) in the presence or in the absence of Lewis acid (2.0 eq) at −78° C., was added the solution of isobutyllithium in hexane (3.0 eq.). The reaction mixture was continued to stir at −78° C. The yield and diastereoselectivity of the addition product were obtained by achiral HPLC method.

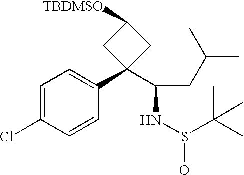

Trans-(1R)-N-{1-[1-(4-chlorophenyl)-3-tert-butyldimethylsiloxy-cyclobutyl]-3-methylbutyl}-(R)-tert-butanesufinamide

This compound was prepared by the above described procedure in 84% yield. ¹H NMR (CDCl₃/TMS): δ 7.28 (d, J=8.3 Hz, 2H), 7.10 (d, J=8.3 Hz, 2H), 4.39 (m, 1H), 3.40-3.22 (m, 2H), 3.01 (d, J=9.9 Hz, 1H), 2.63 (m, 1H), 2.31 (dd, J=7.2, 11.7 Hz, 1H), 2.16 (dd, J=7.2, 12.2 Hz, 1H), 1.60 (m, 1H), 1.20-0.60 (m, 8H), 1.19 (s, 9H), 0.84 (s, 9H), 0.03 (s, 6H). ¹³C NMR (CDCl₃): δ 144.3, 132.1, 129.6, 128.1, 62.9, 62.4, 57.0, 44.2, 43.5, 43.2, 42.0, 25.9, 24.4, 24.1, 23.1, 21.2, 18.1,-4.5,-4.6.

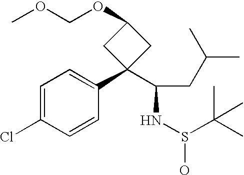

Trans-(1R)-N-{1-[1-(4-chlorophenyl)-3-methoxymethoxy-cyclobutyl]-3-methylbutyl}-(R)-tert-butanesulfinamide

To the solution of MOM protected trans-sulfinamide 19 (4.77 g, 13.3 mmol) in THF (170 mL) at −78° C. under an argon atmosphere, was added the solution of isobutyllithium in hexane (23.1 mL, 1.15 M, 26.6 mmol). After the reaction mixture was stirred at −78° C. for 1 h, TLC showed the reaction was complete. The reaction was then quenched with methanol (10 mL) and water (10 mL) at −78° C. The cold bath was removed and the reaction mixture was allowed to warm to room temperature. The reaction mixture was washed with brine and dried over magnesium sulfate. HPLC showed the ratio of the products was 99.3:0.7. After solvent was removed, the residue was purified by silica gel chromatography eluting with 30% ethyl acetate in heptane to give 21: 5.53 g, 100% yield. ¹H NMR (CDCl₃/TMS): δ 7.29 (d, J=8.5 Hz, 2H), 7.12 (d, J=8.5 Hz, 2H), 4.58 (m, 2H), 4.31 (m, 1H), 3.45-3.25 (m, 2H), 3.33 (s, 3H), 2.97 (d, J=10.1 Hz, 1H), 2.65 (m, 1H), 2.39 (dd, J=7.2, 12.1 Hz, 1H), 2.25 (dd, J=7.1, 12.6 Hz, 1H), 1.61 (m, 1H), 1.36-0.70 (m, 2H), 1.18 (s, 9H), 0.91 (d, J=6.5 Hz, 3H), 0.83 (d, J=6.7 Hz, 3H). ¹³C NMR (CDCl₃): δ 143.9, 132.3, 129.6, 128.3, 95.1, 66.8, 62.5, 57.1, 55.7, 44.0, 41.9, 41.4, 40.5, 24.3, 24.2, 23.1, 21.2. Anal. Calcd for C₂₁H₃₄ClNO₃S: C, 60.63; H, 8.24; N, 3.37. Found: C, 60.54; H, 8.24; N, 3.17.

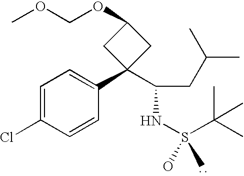

Trans-(1S)-N-{1-[1-(4-chlorophenyl)-3-methoxymethoxy-cyclobutyl]-3-methylbutyl}-(S)-tert-butanesulfinamide

To the solution of MOM protected trans-sulfinamide (4.44 g, 12.4 mmol) in THF (150 mL) at −78° C. under an argon atmosphere, was added the solution of isobutyllithium in hexane (23.7 mL, 1.15 M, 27.3 mmol). After the reaction mixture was stirred at −78° C. for 1 h, TLC showed the reaction was complete. The reaction was then quenched with methanol (10 mL) and water (10 mL) at −78° C. The cold bath was removed and the reaction mixture was allowed to warm to room temperature. The reaction mixture was washed with brine and dried over magnesium sulfate. HPLC showed the ratio of the products was 99.8:1.2. After solvent was removed, the residue was purified by silica gel chromatography eluting with 30% ethyl acetate in heptane to give 23: 4.86 g, 94% yield. ¹H NMR and ¹³C NMR spectra are identical to those of enantiomer 21. Anal. Calcd for C₂₁H₃₄ClNO₃S: C, 60.63; H, 8.24; N, 3.37. Found: C, 60.47; H, 8.33; N, 3.06.

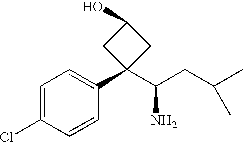

Trans-(1R)-3-(1-amino-3-methyl-butyl)-3-(4-chlorophenyl)-cyclobutanol

The mixture of 21 (2.00 g, 4.8 mmol) with 2 N HCl in methanol (10 mL) was heated to reflux for 30 min. After it was cooled down, the solvent was removed and the residue was dissolved into water (150 mL), washed with TBME (30 mL). The aqueous phase was basified with sodium hydroxide (1.2 g, 30 mmol), extracted with TBME (3×50 mL). The TBME solution was dried over magnesium sulfate. After the solvent was removed, the crude product with D-tartaric acid (0.72 g, 4.8 mmol) was dissolved into methanol (10 mL) and toluene (20 mL). After methanol was evaporated, white solid was precipitated out. The solid was filtered, rinsed with hexane (2×10 mL) and dried under vacuum. 1.69 g (82% yield) of the D-tartaric acid salt was obtained. ¹H NMR (DMSO): δ 7.40 (d, J=7.8 Hz, 2H), 7.19 (d, J=7.8 Hz, 2H), 6.04 (brs, 9H), 4.23 (m, 1H), 3.95 (s, 2H), 3.27 (d, J=10.3 Hz, 1H), 2.95 (m, 1H), 2.65 (m, 1H), 2.15-1.95 (m, 2H), 1.62 (m, 1H), 1.28 (t, J=12.7 Hz, 1H), 0.93 (t, J=11.6 Hz, 1H), 0.86 (d, J=6.2 Hz, 3H), 0.81 (d, J=6.4 Hz, 3H). ¹³C NMR (DMSO): o 174.3, 143.8, 131.1, 129.3, 127.9, 71.6, 60.4, 55.1, 42.9, 42.4, 41.1, 38.3, 23.8, 23.4, 20.9. Anal. Calcd for C₁₉H₂,ClNO₇-1.5 H₂O: C, 52.90; H, 6.89; N, 3.25. Found: C, 53.1 1;H, 6.81;N, 3.1 1.

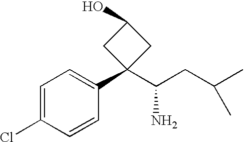

Trans-(1S)-3-(1-amino-3-methyl-butyl)-3-(4-chlorophenyl)-cyclobutanol

The mixture of 32 (1.87 g, 4.5 mmol) with 2 N HCl in methanol (10 mL) was heated to reflux for 30 min. After it was cooled down, the solvent was removed and the residue was dissolved into water (150 mL), washed with TBME (30 mL). The aqueous phase was basified with sodium hydroxide (1.2 g, 30 mmol), extracted with TBME (3×50 mL). The TBME solution was dried over magnesium sulfate. After the solvent was removed, the crude product with L-tartaric acid (0.68 g, 4.5 mmol) was dissolved into methanol (10 mL) and toluene (20 mL). After methanol was evaporated, white solid was precipitated out. The solid was filtered, rinsed with hexane (2×10 mL) and dried under vacuum. 1.56 g (80% yield) of the L-tartaric acid salt was obtained. The ¹H NMR and ¹³C NMR data are identical to those of 1. Anal. Calcd for C₁₉H₂₈ClNO₇-0.75H₂O: C, 52.90; H, 6.89; N, 3.25. Found: C, 52.91; H, 6.86; N, 2.96.

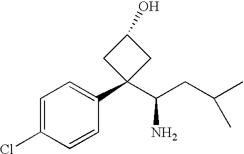

Cis-(1R)-3-(1-amino-3-methyl-butyl)-3-(4-chlorophenyl)-cyclobutanol

The solution (2.08 g, 5.6 mmol) in methanol (10 mL) was treated with 2 N HCl in methanol (15 mL). The reaction mixture was stirred at room temperature for 22 h. The methanol was removed and the residue was dissolved into water (150 mL), washed with TBME (30 mL). The aqueous phase was basified with sodium hydroxide (2.0 g, 50 mmol), extracted with TBME (3×50 mL). The TBME solution was dried over magnesium sulfate. After the solvent was removed, the crude product with L-tartaric acid (0.84 g, 5.6 mmol) was dissolved into methanol (10 mL). After the mixture was stirred at room temperature for 30 min, TBME (10 mL) was added and the mixture was stirred at room temperature for additional 30 min. The white solid was filtered, rinsed with hexane (2×10 mL) and dried under vacuum. 1.87 g (82% yield) of the L-tartaric acid salt was obtained. ¹H NMR (DMSO): δ 7.42 (m, 4H), 6.35 (brs, 7H), 3.93 (s, 2H), 3.73 (m, 1H), 3.34 (d, J=9.3 Hz, 1H), 2.80-2.60 (m, 2H), 2.20 (dd, J=7.4, 10.9 Hz, 1H), 2.03 (dd, J=7.8, 10.9 Hz, 1H), 1.59 (m, 1H), 1.11 (t, J=13.1 Hz, 1H), 0.90-0.60 (m, 7H). ¹³C NMR (DMSO): δ 174.3, 140.2, 131.4, 130.4, 128.1, 71.5, 60.4, 58.0, 43.5, 43.2, 41.5, 38.0, 23.6, 23.2, 21.1. Anal. Calcd for C₁₉H₂₈ClNO₇: C, 54.61; H, 6.75; N, 3.35. Found: C, 54.58; H, 6.79; N, 3.44.

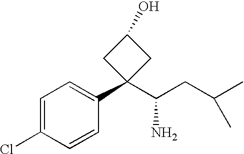

Cis-(1S)-3-(1-amino-3-methyl-butyl)-3-(4-chlorophenyl)-cyclobutanol

The compound (2.08 g, 5.6 mmol) in methanol (15 mL) was treated with 2 N HCl in methanol (20 mL). The reaction mixture was stirred at room temperature for 22 h. The methanol was removed and the residue was dissolved into water (150 mL), washed with TBME (30mL). The aqueous phase was basified with sodium hydroxide (2.0 g, 50 mmol), extracted with TBME (3×50 mL). The TBME solution was dried over magnesium sulfate. After the solvent was removed, the crude product with D-tartaric acid (0.84 g, 5.6 mmol) was dissolved into methanol (10 mL). After the mixture was stirred at room temperature for 30 min, TBME (10 mL) was added and the mixture was stirred at room temperature for additional 30 min. The white solid was filtered, rinsed with hexane (2×10 mL) and dried under vacuum. 1.96 g (85% yield) of the D-tartaric acid salt was obtained. ¹H NMR and ¹³C NMR data are identical to those of 3. Anal. Calcd for C₁₉H₂₈ClNO₇: C, 54.61; H, 6.75; N, 3.35. Found: C, 54.31; H, 6.82; N, 3.29.

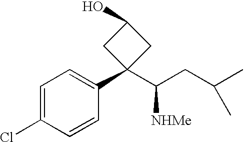

Trans-(1R)-3-(1-methylamino-3-methyl-butyl)-3-(4-chlorophenyl)-cyclobutanol

Typical procedure for the preparation 7-OH DMS: The crude amino alcohol, which was prepared from the deprotection of 30 (2.60 g, 6.2 mmol), was dissolved into toluene (30 mL). To the resulting solution, was added formic acid (2.85 g, 62 mmol). The reaction mixture was then heated to reflux for 5 h. After the solvent was removed, the residue was mixed with borane-THF (20 mL, 1.0 M in THF, 20.0 mmol) at 0° C. After the reaction mixture was stirred at room temperature for 24 h, the reaction was quenched with 2 N HCl (10 mL), diluted with water (150 mL). After separation, the aqueous phase was basified with KOH, extracted with TBME (3×50 mL). The organic layers were combined and dried over magnesium sulfate. The solvent was removed and the residue was dried under vacuum to give crude amino alcohol: 1.33 g in 76% yield. The crude product (1.33 g, 4.7 mmol) with (R)-mandelic acid (0.715 g, 4.7 mmol) was dissolved into methanol (5 mL). After the mixture was stirred at room temperature for 30 min, TBME (10 mL) was added and white solid was precipitated out. The solid was filtered, rinsed with TBME (10 mL), hexane (10 mL) and dried under vacuum to give 1.60 g (78% yield) of the (R)-mandelic acid salt. ¹H NMR (DMSO): δ 7.60-7.00 (m, 9H), 4.75 (s, 1H), 4.70 (brs, 5H), 4.25 (m, 1H), 2.95-2.80 (m, 2H), 2.63 (m, 1H), 2.47 (s, 3H), 2.10-1.90 (m, 2H), 1.54 (m, 1H), 1.23 (m, 1H), 1.0-0.6 (m, 7H). ¹³C NMR (DMSO): δ 174.3, 145.6, 142.1, 130.6, 129.1, 127.7, 126.8, 126.4, 73.0, 63.4, 60.8, 43.2, 42.5, 35.4, 25.5, 23.5, 21.6. Anal. Calcd for C₂₄H₃₂ClNO₄-0.5 H₂O: C, 65.07; H, 7.51; N, 3.16. Found: C, 64.90; H, 7.38; N, 3.07.

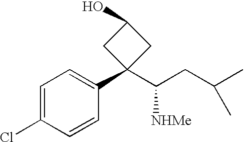

Trans-(1S)-3-(1-methylamino-3-methyl-butyl)-3-(4-chlorophenyl)-cyclobutanol

The crude amino alcohol 32 (0.96 g, 52% yield), which was prepared from 38 (2.71 g, 6.5 mmol) via the typical procedure outlined above, was mixed with (S)-mandelic acid (0.52 g, 3.4 mmol) in methanol (5 mL). After the mixture was stirred at room temperature for 30 min, TBME (10 mL) was added and white solid was precipitated out. The solid was filtered, rinsed with TBME (10 mL), hexane (10 mL) and dried under vacuum to give 1.15 g (78% yield) of the (S)-mandelic acid salt. The ¹H NMR and ¹³C NMR data are identical to those of the (R)-mandelic acid salt of enantiomer. Anal. Calcd for C₂₄H₃₂ClNO₄: C, 66.42; H, 7.43; N, 3.23. Found: C, 66.07; H, 7.38; N, 3.07.

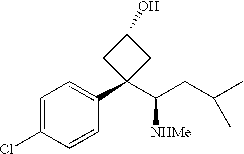

Cis-(1R)-3-(1-methylamino-3-methyl-butyl)-3-(4-chlorophenyl)-cyclobutanol

The crude amino alcohol (2.17 g, 84% yield), which was prepared (3.41 g, 9.16 mmol) via the typical procedure outlined above, was mixed with (R)-mandelic acid (1.065 g, 7.0 mmol) in methanol (5 mL). After the mixture was stirred at room temperature for 30 min, TBME (10 mL) was added and white solid was precipitated out. The solid was filtered, rinsed with TBME (10 mL), hexane (10 mL) and dried under vacuum to give 2.32 g (77% yield) of the (R)-mandelic acid salt. ¹H NMR (DMSO): δ 7.40-7.20 (m, 9H), 5.90 (brs, 4H), 4.76 (s, 1H), 3.67 (m, 1H), 2.86 (m, 1H), 2.79-2.59 (m, 2H), 2.47 (s, 3H), 2.19 (dd, J=8.4, 10.6 Hz, 1H), 2.03 (dd, J=8.1, 10.6 Hz, 1H), 1.59 (m, 1H), 1.01 (m, 1H), 0.9-0.6 (m, 7H). ¹³C NMR (DMSO): δ 174.2, 142.0, 141.7, 130.8, 130.3, 127.7, 126.8, 126.4, 72.9, 66.7, 61.0, 43.9, 43.5, 42.4, 38.6, 34.8, 24.4, 23.2, 22.0. Anal. Calcd for C₂₄H₃₂ClNO₄: C, 66.42; H, 7.43; N, 3.23. Found: C, 66.29; H, 7.47; N, 3.13.

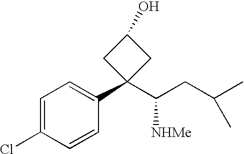

Cis-(1S)-3-(1-methylamino-3-methyl-butyl)-3-(4-chlorophenyl)-cyclobutanol

The crude amino alcohol 32 (2.01 g, 67% yield), which was prepared (3.97 g, 10.7 mmol) via the typical procedure outlined above, was mixed with (S)-mandelic acid (1.02 g, 6.7 mmol) in methanol (5 mL). After the mixture was stirred at room temperature for 30 min, TBME (10 mL) was added and white solid was precipitated out. The solid was filtered, rinsed with TBME (10 mL), hexane (10 mL) and dried under vacuum to give 2.25 g (78% yield) of the (S)-mandelic acid salt. The ¹H NMR and ¹³C NMR data are identical to those of the (R)-mandelic acid salt of entiomer 7. Anal. Calcd for C₂₄H₃₂ClNO₄-0.5 H₂O: C, 65.07; H, 7.51; N, 3.16. Found: C, 65.33; H, 7.28; N, 3.04.

5.1.3. 3-Hydroxyl Experimental Data

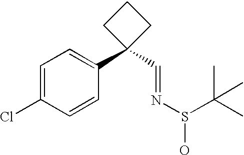

(R)-N-(1-(4-Chlorophenyl)cyclobutanemethylene)-t-butanesulfinamide

To a solution of 1-(4-chlorophenyl)cyclobutanecarbaldehyde (10.0 g, 51.0 mmol), (R)-t-butansulfinamide (5.0 g, 41.0 mmol) in THF (60 mL), Ti(OEt)₄ (46.8 g, 205 mmol) was added and the reaction mixture was stirred at room temperature for 2 h. The reaction mixture was poured into ice-water and the solid was filtered off. The product was extracted with ethyl acetate and the solution was dried over magnesium sulfate. After the solvent was removed, the residue was purified by silica gel chromatography eluting with heptane/ethyl acetate=8.5/1.5(v/v) to give product 12.02 g in 98.5% yield. ¹H NMR (CDCl₃/TMS): δ 8.03 (s, 1H), 7.30 (d, J=8.4 Hz, 2H), 7.10 (d, J=8.4 Hz, 2H), 2.85-2.60 (m, 2H), 2.60-2.40 (m, 2H), 2.15-1.85 (m, 2H), 1.19 (s, 9H). ¹³C NMR (CDCl₃): δ 170.6, 142.5, 132.5, 128.7, 127.5, 57.0, 51.8, 31.1, 30.8, 22.3, 15.9. Anal. Calcd for C₁₅H₂₀ClNOS: C, 60.49; H, 6.77; N, 4.70. Found: C, 60.61; H, 6.80; N, 4.64.

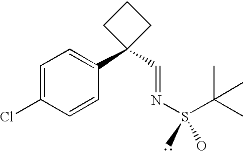

(S)-N-(1-(4-Chlorophenyl)cyclobutanemethylene)-t-butanesulfinamide

11.36 g (93% yield) was prepared via the procedure described for the preparation of imine (R)-sulfinamide (7). ¹H NMR and ¹³C NMR data of 8 are identical to those above.

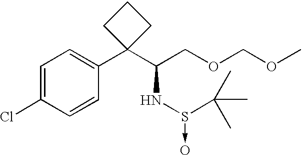

(1S)-N-{1-[1-(4-Chlorophenyl)-cyclobutyl]-2-methoxymethoxyethyl}-(R)-tert-butanesulfinamide

To the solution of (methoxymethoxymethyl)-tri-n-butylstannane (4.02 g, 11 mmol) in THF (20 mL) under an argon atmosphere at −78° C., was added n-BuLi (6.88 mL, 1.6 M in hexane, 11 mmol). After the mixture was stirred at −78° C. for 5 min., the organolithium solution was transferred via a double-ended needle into the solution of (R)-imine sulfinamide (7) (2.98 g, 10 mmol) in THF (20 mL) at −78° C. The reaction mixture was stirred at −78° C. for 2 h. HPLC analysis showed the selectivity of the reaction is 88:12. The reaction was quenched with methanol (1 mL) and water (3 mL). After warmed to room temperature, the reaction mixture was washed with brine and dried over magnesium sulfate. The solvent was removed and the residue was isolated by silica gel chromatography, eluting with 40% ethyl acetate in heptane to give the addition product 9: 3.04 g in 81% yield. ¹H NMR (CDCl₃/TMS): δ 7.31 (d, J=8.5 Hz, 2H), 7.19 (d, J=8.5 Hz, 2H), 4.56 (d, J=6.5 Hz, 1H), 4.51 (d, J=6.5 Hz, 1H), 3.80-3.70 (m, 1H), 3.56 (dd, J=10.4, 4.0 Hz, 1H), 3.33 (s, 3H), 3.11 (d, J=9.0 Hz, 1H), 2.94 (dd, J=10.3, 7.4 Hz, 1H), 2.78-2.65 (m, 1H), 2.48-2.32 (m, 3H), 2.10-1.95 (m, 1H), 1.90-1.75 (m, 1H), 1.17 (s, 9H). ¹³C NMR (CDCl₃): δ 142.9, 132.4, 129.2, 128.4, 96.9, 69.1, 63.8, 56.6, 55.6, 49.1, 33.5, 32.3, 22.8, 15.8. Anal. Calcd for C₁₈H₂₈ClNO₃S: C, 57.82; H, 7.55; N, 3.75. Found: C, 57.67; H, 7.60; N, 3.59.

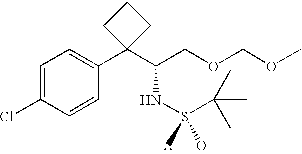

(1R)-N-{1-[1-(4-Chlorophenyl)-cyclobutyl]-2-methoxymethoxyethyl}-(S)-tert-butanesulfinamide

To the solution of (methoxymethoxymethyl)-tri-n-butylstannane (4.38 g, 12 mmol) in THF (20 mL) under an argon atmosphere at −78° C., was added n-BuLi (7.5 mL, 1.6 M in hexane, 12 mmol). After the mixture was stirred at −78° C. for 5 min., the solution of (S)-imine sulfinamide (8) (2.98 g, 10 mmol) in THF (20 mL) at −78° C. The reaction mixture was stirred at −78° C. for 2 h. HPLC analysis showed the selectivity of the reaction is 86:14. The reaction was quenched with methanol (1 mL) and water (3 mL). After usual work-up, the addition product 11 (3.14 g, 84% yield) was isolated by silica gel chromatography, eluting with 40% ethyl acetate in heptane. ¹H NMR and ¹³C NMR data are identical to those of 9. Anal. Calcd for C₁₈H₂₈ClNO₃S: C, 57.82; H, 7.55; N, 3.75. Found: C, 57.92; H, 7.61; N, 3.59.

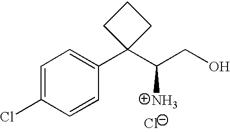

(2S)-2-Amino-2-[1-(4-chloro-phenyl)-cyclobutyl]-ethanol Hydrochloride

To the solution of sulfinamide (3.66 g, 9.8 mmol) in methanol (20 mL), was added the solution of 5-6 N HCl in isopropanol (10 mL). The reaction mixture was heated to reflux for 1 h. HPLC checked the deprotection was complete. After the solvent was removed, the residue was basified with aqueous 2 N NaOH, extracted with TBME. The organic layers were combined and dried over magnesium sulfate. After solvent was removed, the residue was purified by silica gel chromatography, eluting with 2% triethylamine in ethyl acetate to give free amino alcohol: 1.68 g (76% yield). The free amino alcohol (1.68 g, 7.4 mmol) was mixed with the solution of 2 N HCl in diethyl ether (5 mL, 10 mmol) and the mixture was stirred at room temperature for 30 min. The white solid was filtered, rinsed with ether (3×5 mL) and dried over vacuum to give product: 1.82 g, 93% yield. ¹H NMR (DMSO/TMS): δ 8.03 (brs, 3H), 7.41 (d, J=8.6 Hz, 2H), 7.31 (d, J=8.6 Hz, 2H), 5.29 (t, J=4.6 Hz, 1H), 3.63-3.50 (m, 1H), 3.50-3.40 (m, 1H), 3.10 (m, 1H), 2.65-2.45 (m, 2H), 2.40-2.20 (m, 2H), 1.94 (m, 1H), 1.72 (m, 1H). ¹³C NMR (DMSO): δ 142.5, 131.1, 129.3, 128.0, 59.3, 58.8, 46.5, 31.6, 30.9, 15.5. Anal. Calcd for C₁₂H₁₇Cl₂NO: C, 54.97; H, 6.54; N, 5.34. Found: C, 55.08; H, 6.46; N, 5.23.

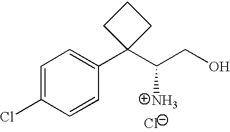

(2R)-2-Amino-2-[1-(4-chloro-phenyl)-cyclobutyl]-ethanol Hydrochloride

To the solution of sulfinamide (2.99 g, 8.0 mmol) in methanol (15 mL), was added the solution of 5-6 N HCl in isopropanol (8 mL). The reaction mixture was heated to reflux for 1 h. HPLC checked the deprotection was complete. After the solvent was removed, the residue was basified with aqueous 2 N NaOH, extracted with TBME. The organic layers were combined and dried over magnesium sulfate. After solvent was removed, the residue was mixed with the solution of 2 N HCl in diethyl ether (8 mL, 10 mmol) and stirred at room temperature for 30 min. The white solid was filtered, rinsed with ether (3×5 mL) and dried over vacuum to give above: 1.86 g, 89% yield. ¹H NMR and ¹³C NMR data are identical to those of 1. Anal. Calcd for C₁₂H₁₇Cl₂NO: C, 54.97; H, 6.54; N, 5.34. Found: C, 54.11; H, 5.65; N, 5.17.

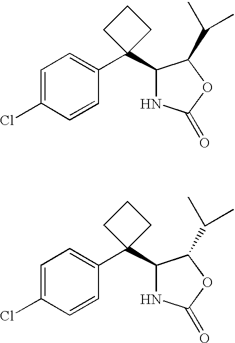

(4S, 5R)-4-[1(4-Chloro-phenyl)-cyclobutyl]-5-isopropyl-oxazolidin-2-one and

(4S, 5S)-4-[1-(4-Chloro-phenyl)-cyclobutyl]-5-isopropyl-oxazolidin-2-one

To the solution of racemic (1-methoxymethoxy-2-methyl-propyl) tri-n-butylstannanes (36.6 g, 90 mmol) in THF (150 mL) at −78° C., was added the solution of n-BuLi in hexane (56.3 mL, 1.6 M, 90 mmol). After the mixture was stirred at −78° C. for 20 min, the solution was transferred and added into the solution of aldimine (14.6 g, 50 mmol) in THF (200 mL) at −78° C. The reaction mixture was continued to stir at −78° C. for 1 h. TLC and HPLC showed no starting material left. The reaction was quenched with methanol (10 mL). The reaction mixture was washed with water (50 mL), saturated sodium chloride (50 mL) and dried over magnesium sulfate. After solvent was removed, the residue was purified by silica gel chromatography, eluting with 25% ethyl acetate in heptane to give a mixture of diastereomers (13): 16.46 g, 79% yield. HPLC showed the ratio of the product of (1S, 2R) to (1S, 2S) was 72:28.

A mixture of 13 (4.80 g, 11.5 mmol) with 2 N HCl in methanol (20 mL) was heated to reflux for 30 min. HPLC showed the deprotection was complete. After solvent was removed, the residue was dissolved into dichloromethane (150 mL). To the resulting solution, was added triethylamine (15 mL), 1,1-carbonyldiimidazole (4.8 g, 30 mmol). The reaction mixture was stirred at room temperature for 1 h. HPLC checked the reaction was complete. The solvent was removed, the residue was isolated by silica gel chromatography, eluting with 25% ethyl acetate in heptane to give the product: (1S, 2R).94 g and (1S, 2S) 0.52 g in 73% combined yield. (1S, 2R): ¹H NMR (CDCl₃/TMS): δ 7.56 (brs, 1H), 7.35 (s, 4H), 4.20-4.05 (m, 2H), 2.60-2.42 (m, 3H), 2.30-2.15 (m, 1H), 2.07-1.85 (m, 3H), 0.92 (d, J=6.5 Hz, 3H), 0.43 (d, J=6.5 Hz, 3H). ¹³C NMR (CDCl₃): δ 161.0, 143.6, 132.4, 128.8, 128.7, 87.6, 63.3, 48.5, 33.8, 28.9, 26.9, 20.0, 19.5, 16.6. Anal. Calcd for C₁₆H₂₀ClNO₂: C, 65.41; H, 6.86; N, 4.77. Found: C, 65.12; H, 7.01; N, 4.60. (1S, 2S): ¹H NMR (CDCl₃/TMS): δ 7.75 (brs, 1H), 7.31 (d, J=8.3 Hz, 2H), 7.11 (d, J=8.3 Hz, 2H), 3.95 (dd, J=3.5, 5.0 Hz, 1H), 3.73 (d, J=3.5 Hz, 1H), 2.50-2.20 (m, 4H), 2.15-1.70 (m, 3H), 0.80 (d, J=6.8 Hz, 3H), 0.77 (d, J=8.8 Hz, 3H). ¹³C NMR (CDCl3): δ 160.7, 143.6, 132.7, 128.7, 128.3, 83.3, 62.4, 48.7, 32.5, 30.8, 28.5, 17.9, 16.1, 15.6. Anal. Calcd for C₁₆H₂₀ClNO₂: C, 65.41; H, 6.86; N, 4.77. Found: C, 65.21; H, 6.86; N, 4.55.

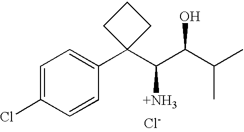

(1S, 2S)-1-Amino-1-[1-(4-chloro-phenyl)-cyclobutyl]-3-methyl-butan-2-ol Hydrochloride

A solution of substituted oxazolidin-2-one 15 (0.343 g, 1.17 mmol), KOH (2.0 g), and NH₂NH₂xH₂O (0.3 mL) in ethylene glycol (10 mL) and water (2 mL) was heated in an 150° C. oil bath for 6 h. HPLC showed the reaction was complete. The reaction mixture was poured into water (20 mL), extracted with TBME (3×20 mL). The organic layer was dried with MgSO₄. After solvent was removed, the residue was mixed with 2 N HCl/ether (3 mL) and stirred at room temperature overnight. The solid was filtered, rinsed with ether (3×3 mL) and dried under vacuum to give product: 0.203 g, 57% yield. ¹H NMR (DMSO/TMS): δ 7.75 (brs, 3H), 7.40 (s, 4H), 5.21 (d? J=6.1 Hz, 1H), 3.35 (brs, 1H), 2.97 (m, 1H), 2.65-2.45 (m, 2H), 2.33 (m, 1H), 2.19 (m, 1H), 1.83 (m, 1H), 1.75-1.60 (m, 2H), 0.78 (d, J=6.5 Hz, 3H), 0.67 (d, J=6.6 Hz, 3H). ¹³C NMR (DMSO): δ 143.3, 131.0, 129.5, 128.1, 71.5, 57.9, 48.0, 31.9, 30.6, 29.7, 19.1, 18.2, 15.5. Anal. Calcd for C₁₅H₂₃Cl₂NO: C, 59.21; H, 7.62; N, 4.60. Found: C, 58.86; H, 7.62; N, 4.52.

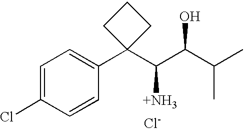

(1S,2R)-1-Amino-i-fl-(4-chloro-phenyl)-cyclobutyl]-3-methyl-butan-2-ol Hydrochloride

A solution of substituted oxazolidin-2-one (1.176 g, 4.0 mmol), KOH (1.71 g), and NH₂NH₂xH₂O (0.25 mL) in ethylene glycol (10 mL) and water (2 mL) was heated in an 120° C. oil bath for 2 h. HPLC showed the reaction was complete. The reaction mixture was poured into water (30 mL), extracted with TBME (3×30 mL). The organic layer was dried with MgSO₄. After solvent was removed, the residue was mixed with 2 N HCl/ether (5 mL) and stirred at room temperature for 30 min. The solid was filtered, rinsed with ether (3×5 mL) and dried under vacuum to give product: 1.02 g, 84% yield. ¹H NMR (DMSO/TMS): δ 7.72 (brs, 3H), 7.59 (d, J=8.3 Hz, 2H), 7.44 (d, J=8.3 Hz, 2H), 4.74 (d, J=6.6 Hz, 1H), 3.38 (brs, 1H), 2.92 (m, 1H), 2.75 (m, 1H), 2.50-2.20 (m, 3H), 1.80 (m, 1H), 1.70-1.48 (m, 2H), 0.83 (d, J=6.5 Hz, 3H), 0.76 (d, J=6.6 Hz, 3H). ¹³C NMR (DMSO): δ 141.1, 131.3, 130.5, 127.8, 73.2, 59.1, 48.4, 34.6, 33.3, 27.9, 20.2, 16.2, 14.5. Anal. Calcd for C₁₅H₂₃Cl₂NO: C, 59.21; H, 7.62; N, 4.60. Found: C, 59.16; H, 7.79; N, 4.50.

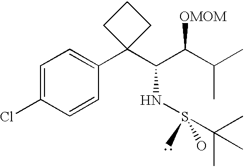

(1R,2S)-N-{1-[1-(4-Chlorophenyl)-cyclobutyl]-2-methoxymethoxy-3-methyl-propyl}-(S) -tert-butanesulfinamide

To the solution of (R)-(1-methoxymethoxy-2-methyl-propyl)-tri-n-butylstannanes (0.92 g, 2.26 mmol) in THF (10 mL) at −78° C., was added the solution of n-BuLi in hexane (1.41 mL, 1.6 M, 2.26 mmol). After the mixture was stirred at −78° C. for 10 min, the solution of aldimine 8 (0.672 g, 2.26 mmol) in THF (5 mL) was added. The reaction mixture was continued to stir at −78° C. for 3 h. HPLC showed about 14% starting material left and the diastereo selectivity of the reaction was 99:1. The reaction mixture was then quenched with methanol (1 mL), washed with saturated sodium chloride (10 mL) and dried over magnesium sulfate. After solvent was removed, the residue was purified by silica gel chromatography, eluting with 25% ethyl acetate in heptane to give product: 0.745 g in 92% yield. ¹H NMR (CDCl₃/TMS): δ 7.44 (d, J=8.7 Hz, 2H), 7.32 (d, J=8.7 Hz, 2H), 4.56 (d, J=6.5 Hz, 1H), 4.50 (d, J=6.5 Hz, 1H), 3.64 (dd, J=4.9, 10.0 Hz, 1H), 3.45 (d, J=10.0 Hz, 1H), 3.28 (s, 3H), 2.95 (dd, J=3.7, 4.8 Hz, 1H), 2.70-2.53 (m, 2H), 2.50-2.48 (m, 2H), 1.92-1.66 (m, 2H), 1.52-1.40 (m, 1H), 1.22 (s, 9H), 0.82 (d, J=6.8 Hz, 3H), 0.81 (d, J=6.6 Hz, 3H). ¹³C NMR (CDCl₃): δ 143.2, 132.3, 130.4, 128.3, 98.6, 87.0, 67.3, 57.1, 56.3, 50.4, 35.7, 33.5, 29.4, 23.0, 21.7, 17.3, 16.7. Anal. Calcd for C₂,H₃₄C1NO₃S: C, 60.63; H, 8.24; N, 3.37. Found: C, 60.46; H, 8.19; N, 3.16.

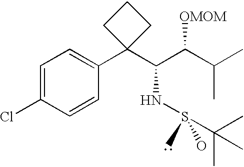

(1R,2R)-N-{1-1-(4-Chlorophenyl)-cyclobutyl]-2-methoxymethoxy-3-methyl-propyl}-(S)-tert-butanesulfinamide

To the solution of (S)-(1-methoxymethoxy-2-methyl-propyl)-tri-n-butylstannanes (0.64 g, 1.6 mmol) in THF (10 mL) at −78° C., was added the solution of n-BuLi in hexane (1.0 mL, 1.6 M, 1.6 mmol). After the mixture was stirred at −78° C. for 10 min, the solution of aldimine 8 (0.47 g, 1.6 mmol) in THF (5 mL) was added. The reaction mixture was continued to stir at −78° C. for 3 h. HPLC showed about 11% starting material left and the diastereo selectivity of the reaction was 99:1. The reaction mixture was then quenched with methanol (1 mL), washed with saturated sodium chloride (10 mL) and dried over magnesium sulfate. After solvent was removed, the residue was purified by silica gel chromatography, eluting with 25% ethyl acetate in heptane to give above product: 0.356 g in 61% yield. ¹H NMR (CDCl₃/TMS): δ 7.36 (d, J=8.7 Hz, 2H), 7.29 (d, J=8.7 Hz, 2H), 4.18 (d, J=6.2 Hz, 1H), 4.04 (d, J=10.3 Hz, 1H), 3.91 (d, J=6.2 Hz, 1H), 3.09 (m, 1H), 3.07 (s, 3H), 2.76-2.64 (m, 1H), 2.47-2.17 (m, 3H), 2.00-1.70 (m, 3H), 1.25 (s, 9H), 0.92 (d, J=6.8 Hz, 3H), 0.86 (d, J=6.8 Hz, 3H). ¹³C NMR (CDCl₃): δ 143.7, 132.1, 129.8, 128.0, 98.0, 82.9, 63.8, 57.3, 55.8, 50.6, 34.3, 32.7, 31.2, 23.4, 19.0, 18.7, 15.5. Anal. Calcd for C₂₁H₃₄ClNO₃S: C, 60.63; H, 8.24; N, 3.37. Found: C, 59.02; H, 8.11; N, 3.08.

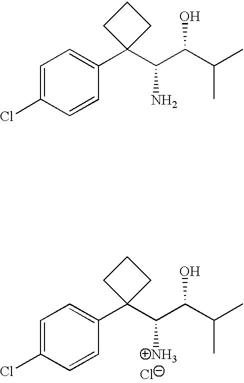

(1R, 2R)-1-Amino-1-[1-(4-chloro-phenyl)-cyclobutyl]-3-methyl-butan-2-ol Hydrochloride

A solution of 17 (0.336 g, 0.81 mmol) with 2 N HCl in methanol (5 mL) was refluxed for 30 min. After solvent was evaporated, the residue was basified with aqueous 2 N NaOH, extracted with TBME (3×10 mL). The organic layer was dried over magnesium sulfate. After solvent was removed, the residue was dried under vacuum to give crude free amino alcohol: ¹H NMR (CDCl₃/TMS): δ 7.29 (d, J=8.4 Hz, 2H), 7.16 (d, J=8.4 Hz, 2H), 3.15 (dd, J=2.0, 6.7 Hz, 1H), 3.06 (d, J=2.0 Hz, 11H), 2.46-2.20 (m, 4H), 2.24-1.75 (m, 2H), 1.74-1.56 (m, 1H), 1.46 (brs, 3H), 0.94-0.86 (m, 6H). ¹³C NMR (CDCl₃): δ 145.3, 131.9, 128.4, 128.3, 74.1, 58.2, 50.2, 32.3, 32.1, 31.5, 19.6, 18.3, 15.8.

The crude free amino alcohol was mixed with 2 N HCl in ether (2 mL). The mixture was stirred at room temperature overnight. The solid was filtered, rinsed with dry ether (3×1 mL) and dried under vacuum to give the HCl salt: 0.22 g in 89% yield. ¹H NMR (DMSO/TMS) and ¹³C NMR (DMSO) data are identical to those of 3: Anal. Calcd for C₁₅H₂₃Cl₂NO: C, 59.21; H, 7.62; N, 4.60. Found: C, 59.33; H, 7.68; N, 4.40.

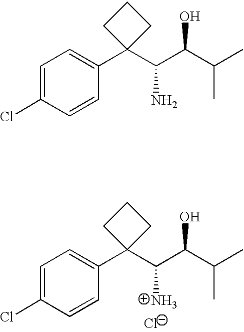

(1R, 2S)-1-Amino-1-[1-(4-chloro-phenyl)-cyclobutyl]-3-methyl-butan-2-ol Hydrochloride

A solution of SM (0.712 g, 1.7 mmol) with 2 N HCl in methanol (5 mL) was refluxed for 30 min. After solvent was evaporated, the residue was basified with aqueous 2 N NaOH, extracted with TBME (3×10 mL). The organic layer was dried over magnesium sulfate. After solvent was removed, the residue was dried under vacuum to give crude free amino alcohol: ¹H NMR (CDCl₃/TMS): δ 7.38 (d, J=8.5 Hz, 2H), 7.30 (d, J=8.5 Hz, 211), 2.95 (d, J=9.4 Hz, 1H), 2.69 (dd, J=2.1, 9.4 Hz, 1H), 2.64-2.26 (m, 4H), 1.98-1.68 (m, 311), 0.88 (d, J=6.8 Hz, 3H), 0.84 (d, J=7.0 Hz, 3H). ¹³C NMR (CDCl₃): d 143.2, 131.8, 129.8, 127.9, 78.2, 61.2, 51.3, 34.7, 33.7, 28.9, 20.3, 16.8, 14.1.

The crude free amino alcohol was mixed with 2 N HCl in ether (3 mL). The mixture was stirred at room temperature overnight. The solid was filtered, rinsed with dry ether (3×1 mL) and dried under vacuum to give the HCl salt: 0.43 g in 83% yield. ¹H NMR (DMSO/TMS) and ¹³C NMR (DMSO) data are identical to those above: Anal. Calcd for C₁₅H₂₃Cl₂NO: C, 59.21; H, 7.62; N, 4.60. Found: C, 59.37; H, 7.59; N, 4.42.

5.2. Pharmacological Assays

The compounds of the invention can readily be tested to demonstrate their utility as pharmaceutical agents. Indeed, certain compounds have been tested. The 1-OH metabolites of N-didesmethylsibutramine (1-OH-DDMS) were tested to determine thir ability to functional uptake of serotonin (5-HT), norepinephrine (NE), or dopamine (DA), into synaptosomes prepared from rat whole brain, hypothalamus, or corpora striata, respectively. In addition, binding was determined at the nonselective muscarinic receptor and the β3-receptor from rat cerebral cortex and rat adipose tissue, respectively. The 1-OH-DDMS compounds tested contain two chiral centers (C2, C4): (2R,4R) (2S,4R), (2R,4S), and (2S,4S).

Compounds were tested initially at 10 μM in duplicate, and if ≧50% inhibition of uptake or binding was observed, they were tested further at 10 different concentrations in duplicate in order to obtain full inhibition or competition curves. IC₅₀ values (concentration inhibiting control activity by 50%) were then determined by nonlinear regression analysis of the inhibition curves and tabulated below.

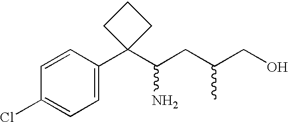

IC₅₀ Values (nM) for 1-OH Metabolites of DDMS in
Functional Uptake Assays

| 1-OH-DDMS | 5-HT | NE | DA |
|---|---|---|---|
| (2R,4R) | 34 | 4.7 | 27 |
| (2S,4S) | 1200 | 150 | 33 |
| (2R,4S) | 2000 | 71 | 110 |
| (2S,4R) | 65 | 11 | 37 |
| Imipramine | 24/24 | | |
| Prtriptyline | | 1.8/4.6 | |
| GBR 12909 | | | 4.2/8.8 |

IC₅₀ values for muscarinic and β3-binding were not calculated because none of the compounds showed inhibition of ≧50%. The maximum inhibition was 13% at the muscarinic site [(2R,4S)-1-OH-DDMS] and 30% at the β3-receptor [(2S,4S)-1-OH-DDMS].

The (R,R)- and (S,R)-hydroxy metabolites are metabolites of (R)-DDMS and the (S,S)- and (R,S)-hydroxy metabolites are metabolites of (S)-DDMS. As a basis of comparison, the IC₅₀ values of (R)-DDMS for inhibition of uptake of 5-HT, NE, and DA were reported to be 140, 13, and 8.9 nM, respectively, and those for (S)-DDMS were 4,300, 62, and 12 nM.

The 1-OH metabolites of N-desmethylsibutramine (DMS) were also tested to determine their ability to inhibit functional uptake of serotonin (5-HT), norepinephrine (NE), or dopamine (DA), into synaptosomes prepared from rat whole brain, hypothalamus, or corpora striata, respectively. In addition, binding was determined at the nonselective muscarinic receptor and the β3-receptor from rat cerebral cortex and rat adipose tissue, respectively. The 1-OH-DMS compounds tested contained two chiral centers (C2, C4): (2R,4R), (2S,4S), (2R,4S), and (2S,4R).

Compounds were tested initially at 10 μM in duplicate, and if ≧50% inhibition of uptake or binding was observed, they were tested further at 10 different concentrations in duplicate in order to obtain full inhibition or competition curves. IC₅₀ values (concentration inhibiting control activity by 50%) were then determined by nonlinear regression analysis of the inhibition curves and tabulated below.

IC₅₀ Values (nM) for 1-OH Metabolites of DMS
in Functional Uptake Assays

| 1-OH-DMS | 5-HT | NE | DA |
|---|---|---|---|
| (2R,4R) | 12 | 2.2 | 19 |
| (2S,4S) | — | 250 | 430 |
| (2R,4S) | — | 74 | 2500 |
| (2S,4R) | 160 | 9.3 | 37 |
| Imipramine | 24 | | |
| Prtriptyline | | 11 | |
| GBR 12909 | | | 8.8 |

— indicates ≦50%
IC₅₀ values for muscarinic and β3-binding were not calculated because none of the compounds showed inhibition of ≧50%. The maximum inhibition was 41% at the muscarinic site [(2S,4S)-1-OH-DMS] and 24% at the β3-receptor [(2S,4R)-1-OH-DMS].

The (2R,4R)- and (2S,4R)-hydroxy metabolites are metabolites of (R)-DMS and the (2S,4S)-(2R,4S)-hydroxy metabolites are metabolites of (S)-DMS. As a basis of comparison, the IC₅₀ values of (R)-DMS for inhibition of uptake of 5-HT, NE, and DA were reported to be 44, 4, and 12 nM, respectively, and those for (S)-DMS were 9,200, 870, and 180 nM.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The embodiments of the invention described above are intended to be merely exemplary and those skilled in the art will recognize, or be able to ascertain using no more than routine experimentation, numerous equivalents to the specific procedures described herein. All such equivalents are considered to be within the scope of the invention and are encompassed by the following claims.

What is claimed is:

1. A compound of the formula: 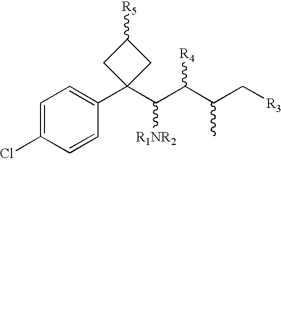
or a pharmaceutically acceptable salt, solvate, hydrate, clathrate, or prodrug thereof, wherein each of R₁ and R₂ is independently lower alkyl or hydrogen, and each of R₃, R₄, and R₅ is independently hydrogen, hydroxyl, or alkoxy, provided that: at least one of R₃, R₄, and R₅ is not hydrogen; if each of R₁, R₂, R₄, and R₅ is hydrogen and R₃ is hydroxyl, the compound is not racemic; and if each of R₁, R₂, R₃, and R₄ is hydrogen and R₅ is hydroxyl, the compound is not racemic.

2. A compound of the formula: 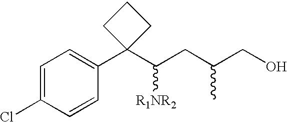
or a pharmaceutically acceptable, salt, solvate, hydrate, clathrate, or prodrug thereof, wherein each of R₁ and R₂ is independently alkyl or hydrogen, provided that if R₁ and R₂, are both hydrogen, the compound is not racemic.

3. A compound of the formula: 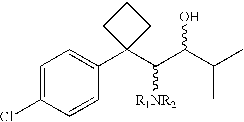
or a pharmaceutically acceptable, salt, solvate, hydrate, clathrate, or prodrug thereof, wherein each of R₁ and R₂ is independently alkyl or hydrogen.

4. A compound of the formula: 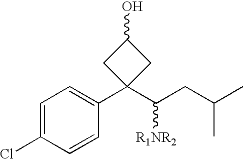
a pharmaceutically acceptable, salt, solvate, hydrate, clathrate, or prodrug thereof, wherein each of R₁ and R₂ is independently alkyl or hydrogen, provided that if both R₁ and R₂ are hydrogen, the compound is not racemic.

5. The compound of claim 1, 2, 3, or 4, wherein at least one of R₁ or R₂ is hydrogen.

6. The compound of claim 1, 2, 3, or 4, wherein at least one of R₁ or R₂ is methyl.

7. The compound of claim 1, 2, 3, or 4, wherein the compound is stereomerically pure.

8. The compound of claim 1, 2, 3, or 4, wherein the compound is an enantiomeric or diastereomeric mixture that is not a racemic mixture.

9. A method of treating or preventing a disease or disorder ameliorated by inhibition of neuronal monoamine uptake, which comprises administering to a patient in need of such treatment or prevention a therapeutically or prophylactically effective amount of a compound of the formula: 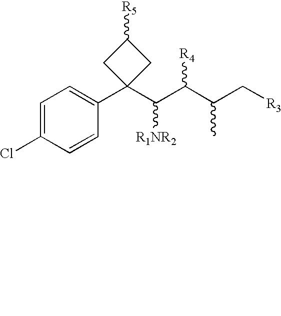
or a pharmaceutically acceptable salt, solvate, hydrate, clathrate, or prodrug thereof, wherein each of R₁ and R₂ is independently lower alkyl or hydrogen, and each of R₃, R₄, and R₅ is independently hydrogen, hydroxyl, or alkoxy, provided that at least one of R₃, R₄, and R₅ is not hydrogen.

10. The method of claim 9, wherein if R₁, R₂, R₄, and R₅ are each hydrogen and R₃ is hydroxyl, the compound is not racemic, and if R₁, R₂, R₃, and R₄ are each hydrogen and R₅ is hydroxyl, the compound is not racemic.

11. The method of claim 9, wherein the disease or disorder ameliorated by inhibition of neuronal monoamine uptake is an eating disorder, an obsessive-compulsive disorder, platelet adhesion, apnea, an affective disorder, anxiety, a male or female sexual function disorder, restless leg syndrome, osteoarthritis, substance abuse, pain, migraine, a cerebral function disorder, a chronic disorder, or incontinence.

12. The method of claim 11 wherein the eating disorder is weight gain or obesity.

13. The method of claim 11 wherein the affective disorder is depression, attention deficit disorder, a bipolar or manic condition, dysthymic disorder, or cyclothymic disorder.

14. The method of claim 11 wherein the pain is neuropathic pain.

15. The method of claim 11 wherein the cerebral function disorder is dementia, memory loss, autism, epilepsy, hyperkinetic syndrome, or schizophrenia.

16. The method of claim 11 wherein the chronic disorder is narcolepsy, chronic fatigue syndrome, seasonal affective disorder, fibromyalgia, or premenstrual syndrome.

17. The method of claim 9, wherein at least one of R₁ or R₂ is hydrogen and at least one of R₃, R₄, or R₅ is hydroxyl.

18. The method of claim 9, wherein at least one of R₁ or R₂ is methyl and at least one of R₃, R₄,or R₅ is hydroxyl.

19. The method of claim 9, wherein the compound is stereomerically pure.

20. The method of claim 9, wherein the compound is an enantiomeric or diastereomeric mixture that is not a racemic mixture.

21. The method of claim 9, wherein the compound is hydroxylated sibutramine or a hydroxylated sibutramine metabolite.

22. The method of claim 21, wherein the compound is hydroxylated in the 1-position.

23. The method of claim 21, wherein the compound is hydroxylated in the 3-position.

24. The method of claim 21, wherein the compound is hydroxylated in the 7-position.

25. The method of claim 9, wherein the amount administered is from about 0.01 mg to about 500 mg/day.

26. The method of claim 25, wherein the amount administered is from about 0.1 mg to about 250 mg/day.

27. The method of claim 25, wherein the amount administered is from about 1 mg to about 100 mg/day.

28. The method of claim 9, wherein the compound is administered orally, mucosally, parenterally, or transdermally.

29. The method of claim 9 further comprising administering a 5-HT₃ antagonist.

30. The method of claim 29, wherein the 5-HT₃ antagonist is an antiemetic agent.

31. The method of claim 29, wherein the 5-HT₃ antagonist is granisetron, metoclopramide, ondansetron, renzapride, zacopride, tropisetron, or a stereomerically pure stereoisomer, active metabolite, or pharmaceutically acceptable salt, solvate, hydrate, ester, clathrate, or prodrug thereof.

32. A pharmaceutical composition comprising a therapeutically or prophylactically effective amount of a racemic or stereomerically pure compound of formula: 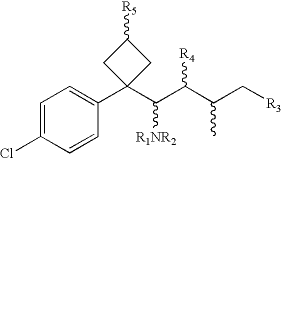
or a pharmaceutically acceptable salt, solvate, hydrate, clathrate, or prodrug thereof, wherein each of R₁ and R₂ is independently lower alkyl or hydrogen, and each of R₃, R₄, and R₅is independently hydrogen, hydroxyl, or alkoxy, provided that at least one of R₃, R₄, and R₅ is not hydrogen.

33. The pharmaceutical composition of claim 32, wherein if R₁, R₂, R₄, and R₅ are each hydrogen and R₃ is hydroxyl, the compound is not racemic, and if R₁, R₂,-R₃, and R₄ are each hydrogen and R₅ is hydroxyl, the compound is not racemic.

34. The pharmaceutical composition of claim 32, wherein at least one of R₁ or R₂ is hydrogen and at least one of R₃, R₄, or R₅ is hydroxyl.

35. The pharmaceutical composition of claim 32, wherein at least one of R₁ or R₂ is methyl and at least one of R₃, R₄, or R₅ is hydroxyl.

36. The pharmaceutical composition of claim 32, wherein the compound is stereomerically pure.

37. The pharmaceutical composition of claim 32, wherein the compound is an enantiomeric or diastereomeric mixture that is not racemic.

38. The pharmaceutical composition of claim 32, wherein the compound is hydroxylated sibutramine or a hydroxylated sibutramine metabolite.

39. The pharmaceutical composition of claim 38, wherein the compound is hydroxylated in the 1-position.

40. The pharmaceutical composition of claim 38, wherein the compound is hydroxylated in the 3-position.

41. The pharmaceutical composition of claim 38, wherein the compound is hydroxylated in the 7-position.

42. The pharmaceutical composition of claim 32, wherein the pharmaceutical composition is adapted for oral, mucosal, rectal, parenteral, or transdermal administration.

43. The pharmaceutical composition of claim 32, wherein said composition is lactose-free.

44. A method of synthesizing a hydroxylated compound, which comprises contacting an aldehyde of formula: 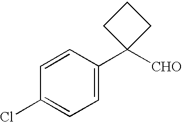
with a first sulfinamide under reaction conditions suitable for the formation of a sulfinimine of formula: 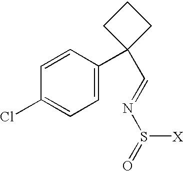
wherein X is an auxiliary group;
contacting said sulfinimine with an organometallic reagent under reaction conditions suitable for the formation of a second sulfinamide of formula: 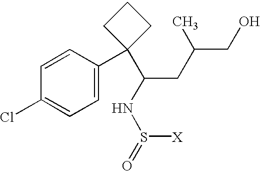
and contacting said second sulfinamide with a reagent under reaction conditions suitable for the removal of a sulfinyl group to form a hydroxylated compound of formula: 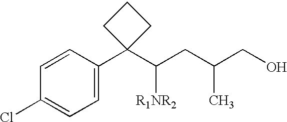
herein each of R₁ and R₂ is independently hydrogen or lower alkyl.

45. The method of claim 44, wherein the hydroxylated compound is stereomerically pure.

46. The method of claim 44, wherein the first sulfinamide is stereomerically pure (R)-tert-butylsulfinamide or stereomerically pure (S)-tert-butylsulfinamide.

47. The method of claim 44, wherein the first sulfinimine is of the formula:

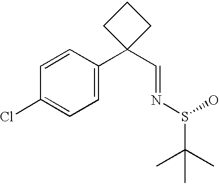

48. The method of claim 44, wherein the organometallic reagent is of the formula:

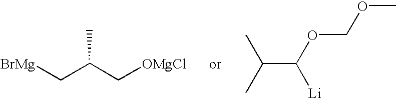

49. The method of claim 44, which further comprises contacting with an N-methylating agent with the hydroxylated compound under reaction conditions suitable for the formation of an N-methyl amine.

50. The method of claim 49, wherein the N-methylating agent is formic acid and borane.

51. The method of claim 44, wherein the organometallic reagent is added under reaction conditions suitable for the formation of a compound of formula: 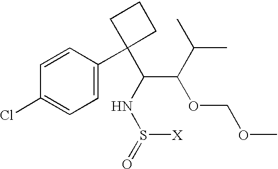
wherein X is an auxiliary group.

52. A method of making a hydroxylated compound of the formula: 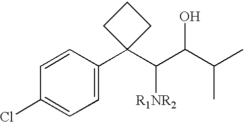
wherein each of R₁ and R₂ is independently hydrogen or alkyl, which comprises treating a first sulfinimine of the formula: 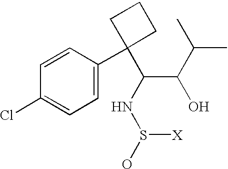
wherein X is an auxiliary with an organometallic agent under reaction conditions suitable for the formation of a second sulfinamide of the formula: 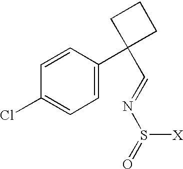
and contacting the second sulfinamide with a deprotecting agent under reaction conditions sufficient for the removal of a sulfinyl group.

53. The method of claim 52, wherein the hydroxylated compound is stereomerically pure.

54. The method of claim 52, wherein the hydroxylated compound is subjected to reaction conditions suitable for N-methylation to form a compound of formula: 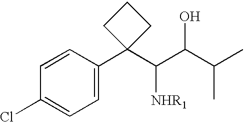
wherein R₁ is methyl.

55. A method for synthesizing a hydroxylated compound, which comprises contacting 4-chlorophenylacetonitrile with a compound under reaction conditions suitable for the formation of a compound of formula: 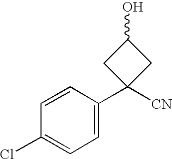
reducing the nitrile under reaction conditions suitable for the formation of an aldehyde;
contacting the aldehyde with a tert-butyl sulfinimine under reaction conditions suitable for the formation of a first sulfinamide compound of formula: 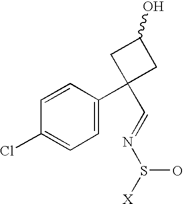
wherein X is an auxiliary;
contacting said first sulfinamide compound with an organometallic agent under reaction conditions suitable for the formation of a second sulfinamide of the formula: 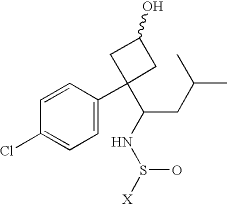
and contacting the second sulfinamide with a deprotecting agent under reaction conditions sufficient to form the hydroxylated compound.

56. The method of claim 55, wherein the hydroxylated compound is stereomerically pure.

57. The method of claim 55, wherein the 4-chlorophenylacetonitrile is contacted with a compound of the formula:

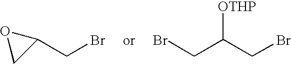

58. The method of claim 55, wherein the reducing agents is Dibal-H.

59. The method of claim 55, wherein the organometallic is isopropylmagnesium chloride.

60. A compound of the formula: 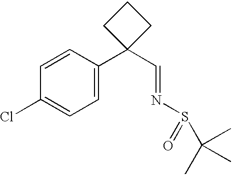
or a salt, solvate, or hydrate thereof.

61. A compound of the formula: 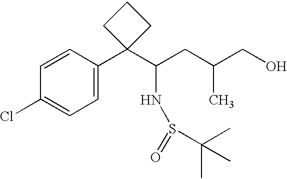
or a salt, solvate, or hydrate thereof.

62. A compound of the formula: 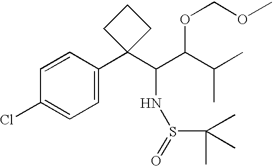
or a salt, solvate, or hydrate thereof.

63. A compound of the formula: 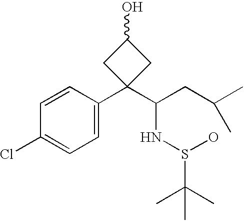
or a salt, solvate, or hydrate thereof.

64. A compound of the formula: 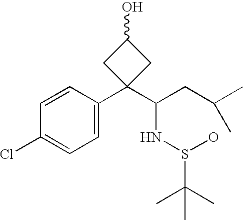
or a salt, solvate, or hydrate thereof.

65. A compound of the formula: 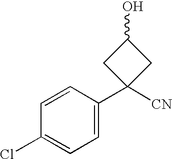
or a salt, solvate, or hydrate thereof.

66. A compound of the formula: 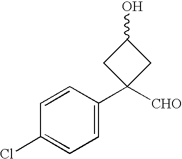
or a salt, solvate, or hydrate thereof.

67. A method of providing a reduced compound, which comprises contacting a compound of the formula: 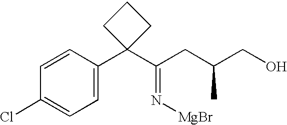
with a borane-reducing agent under reaction conditions suitable for the formation of a stereomerically pure compound of the formula:

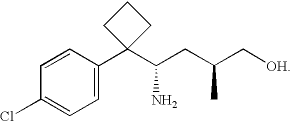

68. A method of providing a reduced compound, which comprises contacting a compound of the formula: 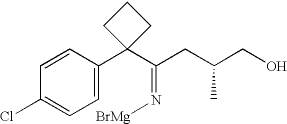
with a borane-reducing agent under reaction conditions suitable for the formation of a stereomerically pure compound of the formula:

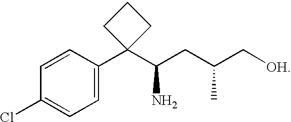

69. The method of claim 67 or 68 wherein the borane reducing agent is formed by contacting borane-tetrahydrofuran with succinic acid.

70. The method of claim 67 or 68, wherein the borane reducing agent is formed by contacting borane-tetrahydrofuran with salicyllic acid.

71. A compound of the formula:

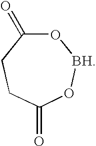

72. A method of synthesizing a sulfinamide of the formula: 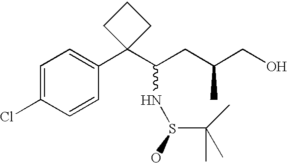
which comprises contacting an organometallic agent of the formula: 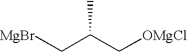
with a compound of the formula: 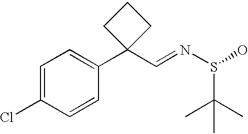
under reaction conditions sufficient for the formation of the sulfinamide.

73. The method of claim 72, wherein the sulfinamide, organmetallic agent, and sulfinimine are stereomerically pure.
